(19) United States
(12) Patent Application Publication
(10) Pub. No.: US 2010/0240656 A1
(43) Pub. Date: Sep. 23, 2010

(54) COMPOUNDS AS HSP90 INHIBITORS

(75) Inventors: Marc Martinell Pedemonte, Barcelona (ES); Isabel Navarro Muñoz, Barcelona (ES); Montserrat Soler López, Barcelona (ES); David Mormeneo Julián, Barcelona (ES); Malgorzata Rosol, Barcelona (ES); Amadeo Llebaria Soldevila, Barcelona (ES); Juan Aymami Bofarull, Barcelona (ES)

Correspondence Address: Workman Nydegger;1000 Eagle Gate Tower, 60 East South Temple, Salt Lake City, UT, 84111, US

(73) Assignee: ORYZON GENOMICS, S.A., Barcelona (ES)

(21) Appl. No.: 12/668,170

(22) PCT Filed: Jul. 9, 2008
(86) PCT No.: PCT/EP08/58924

§ 371 Date: Jun. 11, 2010

(30) Foreign Application Priority Data
Jul. 12, 2007 (EP) 07112388.9

(51) Int. Cl. A61K 31/5355; C07D 209/42; C07D 473/00; C07D 233/96; C07D 277/36; C07D 401/12; C07D 413/14; C07D 231/38; A61K 31/40; A61K 31/52; A61K 31/4155; A61K 31/428; A61K 31/4439; A61P 35/00; A61P 9/10; A61P 17/06; A61P 19/00

(52) U.S. Cl. 514/235.8; 548/492; 544/264; 548/311.7; 548/171; 546/274.4; 544/139; 548/364.4; 514/419; 514/263.1; 514/397; 514/367; 514/341; 514/407

(57) ABSTRACT

The invention provides novel compounds of formula (I) wherein: one of the a, b, c or d members is a nitrogen atom and the remaining members are carbon atoms; and R₃ is a radical selected from the group consisting of: —S—R₁₄ and —CH₂—R₁₅. The compounds of formula (I) are useful for treating diseases mediated by a heat shock protein 90 (Hsp 90).

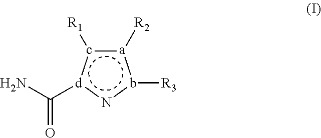

The present invention is related to the fields of chemistry and molecular pathology. More particularly, the present invention refers to heterocyclic compounds which are useful as heat shock protein 90 inhibitors and hence are useful in the treatment of conditions mediated by Hsp90 including, for example, cancer.

### BACKGROUND ART

The heat shock protein 90 (hereinafter abbreviated as “Hsp90”) constitutes about 1-2% of total cellular protein, and is usually present in the cell as a dimer in association with a number of other proteins. It is essential for cell viability and exhibits dual chaperone functions. It plays a key role in the cellular stress response by interacting with many proteins (named client proteins) after their native conformation has been altered by various environmental stresses, such as heat shock, thus ensuring adequate protein folding and preventing non-specific aggregation. In addition, it has been suggested that Hsp90 may also play a role in buffering against the effects of mutation, presumably by correcting the inappropriate folding of mutant proteins. Furthermore, Hsp90 also has an important regulatory role. Under normal physiological conditions, together with its endoplasmic reticulum homologue GRP94, Hsp90 plays a housekeeping role in the cell, maintaining the conformational stability and maturation of several key client proteins. Hsp90 inhibitors lock the chaperone cycle thus preventing the formation of mature chaperone complexes and driving proteasome-mediated degradation of its client proteins. These client proteins can be subdivided into three groups: (a) steroid hormone receptors, (b) Ser/Thr or tyrosine kinases (e.g., ERBB2, RAF-1, CDK4, and LCK), and (c) a collection of apparently unrelated proteins, e.g., mutant p53 and the catalytic subunit of telomerase hTERT. All of these proteins play key regulatory roles in many physiological and biochemical processes in the cell.

Owing to their regulatory implications and their importance in the cell viability, Hsp90 proteins have become an important target for the screening of new compounds for their therapeutic use in several human diseases such as those related to malignancy, central nervous system, immune system, and musculoskeletal system, among others. (cf., Pacey, S. et al., “Hsp90 inhibitors in the clinic”, HEP, 2006, vol. 172: 331-358).

The first class of Hsp90 inhibitors to be discovered was the benzoquinone ansamycin class, which includes the compounds herbimycin A and geldanamycin. However, it was not until the 1980s that their potential application as antitumour agents was discovered. They were shown to reverse the malignant phenotype of fibroblasts transformed by the v-Src oncogene, and to subsequently exhibit potent antitumour activity in both in vitro and in vivo animal models.

Geldanamycin showed activity in human tumour xenograft models but progression of this compound to clinical trial was halted due to unacceptable levels of hepatotoxicity observed at doses required for therapeutic activity. A range of geldanamycin analogues has already been described.

In spite of some Hsp90 inhibitors are known, there is still an important need to find novel compounds.

### SUMMARY OF THE INVENTION

The inventors of the present invention have found that the compounds of formula (I) of the present invention can bind to the Hsp90 and hence inhibit its function. Particularly, the compounds of the invention can optimally interact with the target protein. Owing to their optimal design the compounds of the invention can improve the affinity against the target protein, being possible avoiding the undesired side-effects related to other known Hsp90 inhibitors. For instance, the dose needed to achieve the therapeutic effect compared with the doses used for other compounds of the state of the art can be reduced either reducing the frequency of administration or the dosage amount, making more comfortable to the patient the therapeutic regimen.

Thus, in one aspect the present invention relates to a compound of general formula (I),

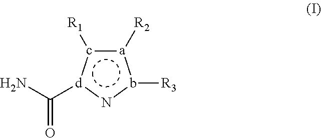

wherein
one of the a, b, c or d members is a nitrogen atom and the remaining members are carbon atoms,
R₁ is hydrogen or a radical selected from the group consisting of: —NR₄R₅; —COR₆; —COOR₆; —C(O)NR₄R₅; —R₉PO(OR₁₀)₂; —NHSO₂—R₁₁; (C₁-C₈)alkyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₁-C₈)-alkyl-Cy; (C₂-C₈)alkenyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₂-C₈)-alkenyl-Cy; (C₂-C₈)alkynyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₂-C₈)-alkynyl-Cy; and a radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₂ is a radical selected from the group consisting of: (C₁-C₈)alkyl; (C₂-C₈)alkenyl; and (C₂-C₈)alkynyl; said radicals being optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, —NR₄R₅, and a radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₃ is a radical selected from the group consisting of: —S—R₁₄ and —CH₂—R₁₅;
   Cy is a known ring systems with 1-4 rings, wherein each one of the rings forming said ring system has from 3 to 7 members, each member independently selected from C, N, O, S, CH, CH₂, and NH, the ring being saturated, partially unsaturated or aromatic, and optionally being substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₁₄ is a C-radical derived from one of the known ring systems with 1-4 rings, wherein each one of the rings:
has 3-7 members, each member independently selected from C, N, O, S, CH, CH₂, NH,
is saturated, partially unsaturated or aromatic, and
is isolated or partially/totally fused
   being each ring forming part of the ring system optionally substituted by at least one radical selected from the group consisting of: halogen, nitro, cyano, —CF₃₃ (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₁₅ is a radical derived from one of the known ring systems with 2-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —CF₃—S—R₁₁, —SO—R₁₁, —SO₂—R₁₁, —NHSO₂—R₁₁, —SO₂—NR₁₂R₁₃, and —NR₄R₅; and
   R₄ and R₅, are radicals, same or different, independently selected from the group consisting of: hydrogen; —COR₆; —COOR₆; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, amino; (C₂-C₈)alkynyl optionally substituted by at least one radical selected from the group consisting of: halogen, nitro, cyano, and amino; and radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is isolated, partially/totally fused;
   being each ring forming part of the ring system optionally substituted by at least one radical selected from the group consisting of: halogen, nitro, cyano, amino, and (C₁-C₈)alkyl;
   R₇ and R₉ are biradicals, same or different, independently selected from the group consisting of: (C₁-C₈)alkyl; (C₂-C₈)alkenyl; and (C₂-C₈)alkynyl; being said radicals optionally substituted by at least one radical selected from the group consisting of: halogen, nitro, cyano, amino, and a known 5- or 6-membered aromatic ring wherein all the members are carbon atoms or wherein from 1 to 4 of the members are selected from N, O, and S, and the remaining members are carbon atoms, being the aromatic ring optionally substituted by at least one radical selected from the group consisting of: halogen, nitro, cyano, amino, (C₁-C₈)alkyl, (C₂-C₈)alkenyl, —OR₆, —COR₆, —COOR₆, —OC(O)R₆, —C(O)NR₄R₅, —S—R₁₁, —SO—R₁₁, —SO₂—R₁₁, —NHSO₂—R₁₁ and —SO₂—NR₁₂R₁₃; and
   R₆, R₈, R₁₀, R₁₁, R₁₂, and R₁₃, are radicals, same or different, independently selected from the group consisting of: hydrogen; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, and amino; (C₂-C₈)alkynyl optionally substituted by at least one radical selected from the group consisting of: halogen, nitro, cyano, and amino; and a radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, and amino.

The inventors have found that the compounds of the invention are able to bind to the ATP binding site of the Hsp90 N-terminal domain and can inhibit the Hsp90 function, owing to the proper combination of the different radicals R₁-R₃, their three-dimensional disposition, as well as to the amide moiety and the fixed nitrogen in the ring. In fact, as it has been elucidated from X-ray data, the amide group and the fixed nitrogen participate in the binding through direct and/or water mediated hydrogen bonds which are essential for the potency and the proper orientation of the molecule. Regarding the chemical nature of the substituents R₁, R₂ and R₃, it has been found that the activity of the compounds is affected especially by the R₃ position which may affect the binding affinity, and hence the activity, by several orders of magnitude.

A second aspect of the invention is a pharmaceutical composition comprising a therapeutically effective amount of a compound of formula (I) according to the first aspect of the invention, together with the appropriate amounts of pharmaceutically acceptable excipients, carriers, or mixtures thereof.

A third aspect of the invention provides a compound according to the first aspect of the invention for use as a medicament.

A fourth aspect of the invention is the use of a compound of formula (I), compound of general formula (I),

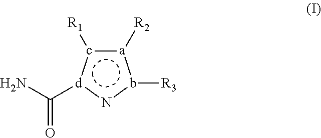

wherein
one of the a, b, c or d members is a nitrogen atom and the remaining members are carbon atoms;
R₁ is hydrogen or a radical selected from the group consisting of: —NR₄R₅; —COR₆; —COOR₆; —C(O)NR₄R₅; —R₉PO(OR₁₀)₂; —NHSO₂—R₁₁; (C₁-C₈)alkyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₁-C₈)-alkyl-Cy; (C₂-C₈)alkenyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₂-C₈)-alkenyl-Cy; (C₂-C₈)alkynyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₂-C₈)-alkynyl-Cy; and a radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₈, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₂ is a radical selected from the group consisting of: (C₁-C₈)alkyl; (C₂-C₈)alkenyl; and (C₂-C₈)alkynyl; said radicals being optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, —NR₄R₅, and a radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₃ is a radical selected from the group consisting of: —S—R₁₄ and —CH₂—R₁₅;
   Cy is a known ring systems with 1-4 rings, wherein each one of the rings forming said ring system has from 3 to 7 members, each member independently selected from C, N, O, S, CH, CH₂, and NH, the ring being saturated, partially unsaturated or aromatic, and optionally being substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₁₄ is selected from the group consisting of: C₁-C₈ alkyl; C₂-C₈alkenyl; C₂-C₈ alkynyl; and a C-radical derived from one of the known ring systems with 1-4 rings, wherein each one of the rings:
has 3-7 members, each member independently selected from C, N, O, S, CH, CH₂, NH,
is saturated, partially unsaturated or aromatic, and
is isolated or partially/totally fused
   being each ring forming part of the ring system optionally substituted by at least one radical selected from the group consisting of: halogen, nitro, cyano, (C₁-C₈)alkyl, —CF₃, (C₂-C₈)alkenyl, —OR₆, —COR₆, —COOR₆, —OC(O)R₆, —C(O)NR₄R₆, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₁₅ is a radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, (C₁-C₈)alkyl, (C₂-C₈)alkenyl, —CF₃, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   R₄ and R₅, are radicals, same or different, independently selected from the group consisting of: hydrogen; —COR₆; —COOR₆; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, amino; (C₂-C₈)alkynyl optionally substituted by at least one radical selected from the group consisting of: halogen, nitro, cyano, and amino; and radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is isolated, partially/totally fused;
   being each ring forming part of the ring system optionally substituted by at least one radical selected from the group consisting of: halogen, nitro, cyano, amino, and (C₁-C₈)alkyl;
   R₇ and R₉ are biradicals, same or different, independently selected from the group consisting of: (C₁-C₈)alkyl; (C₂-C₈)alkenyl; and (C₂-C₈)alkynyl; being said radicals optionally substituted by at least one radical selected from the group consisting of: halogen, nitro, cyano, amino, and a known 5- or 6-membered aromatic ring wherein all the members are carbon atoms or wherein from 1 to 4 of the members are selected from N, O, and S, and the remaining members are carbon atoms, being the aromatic ring optionally substituted by at least one radical selected from the group consisting of: halogen, nitro, cyano, amino, (C₁-C₈)alkyl, (C₂-C₈)alkenyl, —OR₆, —COR₆, —COOR₆, —OC(O)R₆, —C(O)NR₄R₅, —S—R₁₁, —SO—R₁₁, —SO₂—R₁₁, —NHSO₂—R₁₁, and —SO₂—NR₁₂R₁₃; and
   R₆, R₈, R₁₀, R₁₁, R₁₂, and R₁₃, are radicals, same or different, independently selected from the group consisting of: hydrogen; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, and amino; (C₂-C₈)alkynyl optionally substituted by at least one radical selected from the group consisting of: halogen, nitro, cyano, and amino; and a radical derived from one of the known ring systems with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, and amino;
   for the manufacture of a medicament for the treatment of a disease mediated by a heat shock protein 90. This aspect of the invention can also be formulated as compound of formula (I) as defined in the fourth aspect of the invention for use in the treatment of a disease mediated by a heat shock protein 90.

### BRIEF DESCRIPTION OF THE DRAWINGS

FIG. 1 is a western blot analysis to determine the protein levels of Hsp90 client proteins (c-Raf-1, Cdk4, and Her2) and the co-chaperone Hsp70 upon treatment with increasing concentrations of example 6 (compound of the invention) and 17AAG (which is known in the state of the art as a degradation promoter). As shown in the western blot, the compound of the invention (example 6) acts in a similar way that 17AAG which implies that the effect observed in cell viability is mediated through inhibition of Hsp90.

### DETAILED DESCRIPTION OF THE INVENTION

In one embodiment of the first aspect of the invention, the compound of formula (I) is one wherein:

one of the a, b, or c members is a nitrogen atom and the remaining members are carbon atoms,
R₁ is hydrogen or a radical selected from the group consisting of: —NR₄R₅; —NHSO₂—R₁₁; (C₁-C₈)alkyl optionally substituted by at least one radical selected from the group consisting of: —COOR₆, —OC(O)R₆, —C(O)NR₄R₅, and —NR₄R₅ and -Cy;
R₂ is a radical selected from the group consisting of: (C₁-C₈)alkyl; (C₂-C₈)alkenyl; and (C₂-C₈)alkynyl; said radicals being optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, —NR₄R₅, and a radical which is a known ring system with 1-2 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, (C₁-C₈)alkyl, —OR₆, and —NR₄R₅;
   R₃ is a radical selected from the group consisting of: —S—R₁₄ and —CH₂—R₁₅;
   Cy is a known ring system with 1-2 rings, wherein each one of the rings forming said ring system has from 3 to 7 members, each member independently selected from C, N, O, S, CH, CH₂, and NH, the ring being saturated, partially unsaturated or aromatic, and optionally being substituted by at least one radical selected from the group consisting of: halogen, (C₁-C₈)alkyl, —OR₆, and —NR₄R₅;
   R₁₄ is a C-radical which is a known ring system with 1-4 rings, wherein each one of the rings:
has 3-7 members, each member independently selected from C, N, O, S, CH, CH₂, NH,
is saturated, partially unsaturated or aromatic, and
is isolated or partially/totally fused
   being each ring forming part of the ring system optionally substituted by at least one radical selected from the group consisting of: halogen, nitro, cyano, (C₁-C₈)alkyl, —CF₃, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
   is a radical which is a known ring system with 2-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, (C₁-C₈)alkyl, (C₂-C₈)alkenyl, —CF₃, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; and
   R₄ and R₅, are radicals, same or different, independently selected from the group consisting of: hydrogen; —COR₆; —COOR₆; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, amino; (C₂-C₈)alkynyl optionally substituted by at least one radical selected from the group consisting of: halogen, nitro, cyano, and amino; and radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is isolated, partially/totally fused;
   being each ring forming part of the ring system optionally substituted by at least one radical selected from the group consisting of: halogen, nitro, cyano, amino, and (C₁-C₈)alkyl;
   R₇ and R₉ are biradicals, same or different, independently selected from the group consisting of: (C₁-C₈)alkyl; (C₂-C₈)alkenyl; and (C₂-C₈)alkynyl; being said radicals optionally substituted by at least one radical selected from the group consisting of: halogen, and amino; and
   R₆, R₈, R₁₀, R₁₁, R₁₂, and R₁₃, are radicals, same or different, independently selected from the group consisting of: hydrogen; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, and amino; (C₂-C₈)alkynyl optionally substituted by at least one radical selected from the group consisting of: halogen, nitro, cyano, and amino; and a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, nitro, cyano, and amino.

In another embodiment of the first aspect of the invention, the compound of formula (I) is one wherein

one of the a or b members is a nitrogen atom and the remaining members are carbon atoms;
R₁ is selected from —NHSO₂—R₁₁ and —NHR₅;
R₂ is a radical selected from the group consisting of: —R₇NHR₈; (C₁-C₈)alkyl; (C₂-C₈)alkenyl; (C₂-C₈)alkynyl; said radical being optionally substituted by at least one radical selected from the group consisting of: hydroxyl, halogen, nitro, cyano, —NH₂, —NH(C₁-C₈)alkyl, —COOH, —OC(O)(C₁-C₈)alkyl, —NR₄R₅, and a radical which is a known ring system with 1 ring, said ring
having 3-7 members, each member independently selected from C, N, O, S, CH, CH₂, NH,
is saturated, partially unsaturated or aromatic,
   being the ring system optionally substituted by at least one radical selected from the group consisting of: halogen, (C₁-C₈)alkyl, and —OR₆;
   R₃ is a radical selected from the group consisting of: —S—R₁₄ and —CH₂—R₁₅; R₄, R₅ is hydrogen or a radical selected from the group consisting of: (C₁-C₈)alkyl optionally substituted by at least one radical selected from the group consisting of: phenyl, halogen, nitro, cyano, and —NH₂; (C₂-C₈)alkenyl optionally substituted by at least one radical selected from the group consisting of: halogen, nitro, cyano, and —NH₂; —COR₈; and a known ring system consisting of one ring which is aromatic, has from 5-6 members, and each member is independently selected from C, N, O, S, CH, CH₂, NH, being the aromatic ring optionally substituted by at least one radical selected from the group consisting of: halogen, nitro, cyano, —NH₂, and (C₁-C₈)alkyl;
   R₆ is a radical selected from the group consisting of: (C₁-C₈)alkyl optionally substituted by one phenyl radical; a known ring system consisting of one ring which is saturated, has from 3-7 members, and each member is independently selected from C, N, O, S, CH, CH₂, NH; and a known ring system consisting of one ring which is aromatic, has from 5-6 members, and each member is independently selected from C, N, O, S, CH, CH₂, NH, being the ring system optionally substituted by at least one radical selected from the group consisting of: halogen, nitro, cyano, —NH₂, and (C₁-C₈)alkyl;
   R₁₄ is a C-radical which is a known ring system with 1-4 rings, wherein each one of the rings:
has 5-6 members, each member independently selected from C, N, O, S, CH, CH₂, NH,
is saturated, partially unsaturated or aromatic, and
is isolated or partially/totally fused;
   being each ring forming part of the ring system optionally substituted by at least one radical selected from the group consisting of: hydroxyl, halogen, nitro, cyano, —NH₂, —COOH, (C₁-C₈)alkyl, (C₁-C₈)alkenyl, (C₁-C₄)alkoxy, —S—R₁₁, —SO₂—R₁₁, and —CF₃;
   R₁₅ is a radical which is a known ring system with 2-4 rings, wherein each one of the rings forming said ring system
has 5-6 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: hydroxyl, halogen, nitro, cyano, —NH₂, —COOH, (C₁-C₈)alkyl, —CF₃, and (C₁-C₈)alkenyl; and
   R₁₁ is (C₁-C₈)alkyl.

In still another embodiment of the first aspect of the invention, the compound of formula (I) is one wherein:

one of the a or b members is a nitrogen atom and the remaining members are carbon atoms;

### R₁ is —NHR₅;

R₂ is a radical selected from the group consisting of: (C₁-C₈)alkyl; (C₂-C₈)alkenyl; (C₂-C₈)alkynyl; said radical being optionally substituted by at least one radical selected from the group consisting of: hydroxyl, halogen, nitro, cyano, —NH₂, —NH(C₁-C₈)alkyl, —COOH, —OC(O)(C₁-C₈)alkyl, and phenyl; R₃ is a radical selected from the group consisting of: —S—R₁₄ and —CH₂—R₁₅; R₅ is hydrogen or a radical selected from the group consisting of: (C₁-C₈)alkyl optionally substituted by at least one radical selected from the group consisting of: halogen, nitro, cyano, and —NH₂; (C₂-C₈)alkenyl optionally substituted by at least one radical selected from the group consisting of: halogen, nitro, cyano, and —NH₂; —COR₆; and a known ring system consisting of one ring which is aromatic, has from 5-6 members, and each member is independently selected from C, N, O, S, CH, CH₂, NH, being the aromatic ring optionally substituted by at least one radical selected from the group consisting of: halogen, nitro, cyano, —NH₂, and (C₁-C₈)alkyl;
R₆ is a radical selected from the group consisting of: (C₁-C₈)alkyl; a known ring system consisting of one ring which is saturated, has from 3-7 members, and each member is independently selected from C, N, O, S, CH, CH₂, NH; and a known ring system consisting of one ring which is aromatic, has from 5-6 members, and each member is independently selected from C, N, O, S, CH, CH₂, NH; being the radical optionally substituted by at least one radical selected from the group consisting of: halogen, nitro, cyano, —NH₂, and (C₁-C₈)alkyl;
R₁₄ is a C-radical which is a known ring system with 1-4 rings, wherein each one of the rings:
has 5-6 members, each member independently selected from C, N, O, S, CH, CH₂, NH,
is saturated, partially unsaturated or aromatic, and
is isolated or partially/totally fused;
   being each ring forming part of the ring system optionally substituted by at least one radical selected from the group consisting of: hydroxyl, halogen, nitro, cyano, —NH₂, —COOH, (C₁-C₈)alkyl, and (C₁-C₈)alkenyl; and
   R₁₅ is a radical which is a known ring system with 2-4 rings, wherein each one of the rings forming said ring system
has 5-6 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: hydroxyl, halogen, nitro, cyano, —NH₂, —COOH, (C₁-C₈)alkyl, and (C₁-C₈)alkenyl. Preferably R₃ is the radical —S—R₁₄, wherein R₁₄ is a known ring system consisting of one ring or consisting of two or three rings, the rings being totally fused. Preferably, R₃ is the radical —CH₂—R₁₅, wherein R₁₅ is a known ring system consists of two or three rings, the rings being totally fused. More preferably, R₁₄ or R₁₅ are selected from the group consisting of: 6-iodobenzo[1,3]dioxol, benzo[1,3]dioxol, trifluoromethylphenyl, and 2,5-dimethoxyphenyl. Preferably, R₂ is selected from the group consisting of: butyl, pentylamine, and (3-isopropylamine)propyl.

In still another embodiment of the first aspect of the invention, the compound of formula (I) is one wherein:

the a member is a nitrogen atom and the remaining members are carbon atoms;

R₂ is a radical selected from the group consisting of: (C₁-C₈)alkyl optionally substituted by at least one radical selected from the group consisting of: —NH₂, —NH(C₁-C₄)alkyl, —NR₄R₅ and a radical which is a known ring system with 1 saturated ring, the ring having 6 members independently selected from N, O, and CH₂,

R₄ is (C₁-C₄)alkyl;
R₅ is hydrogen or a radical selected from the group consisting of: (C₁-C₄)alkyl optionally substituted by one phenyl radical; —C(O)(C₁-C₄)alkyl optionally substituted by one phenyl radical; —C(O)cyclopropyl; and —SO₂CH₃;
R₃ is a radical selected from the group consisting of: —S—R₁₄ and —CH₂—R₁₅ R₁₄ is a C-radical which is a known ring system with 1-2 rings, wherein each one of the rings:
has 5-6 members, each member independently selected from C, N, O, S, CH, CH₂, NH,
is saturated, partially unsaturated or aromatic, and
is partially/totally fused;
   being each ring forming part of the ring system optionally substituted by at least one radical selected from the group consisting of: halogen, (C₁-C₄)alkyl (C₁-C₄)alkenyl, (C₁-C₄)alkoxy, —S—CH₃, —SC₂—CH₃, and —CF₃; and
   R₁₅ is a radical which is a known ring system with 2 rings totally fused, wherein each one of the rings:
has 5-6 members, each member independently selected from C, O, CH, and, CH₂, and
is saturated, partially unsaturated or aromatic;
   being each ring forming part of the ring system optionally substituted by at least radical selected from the group consisting of: halogen, (C₁-C₄)alkyl (C₁-C₄)alkenyl, (C₁-C₄)alkoxy, —S—CH₃, —SC₂—CH₃, and —CF₃.

In still yet another embodiment of the first aspect of the invention, the compound of formula (I) is one wherein:

the b member is a nitrogen atom and the remaining members are carbon atoms;
R₂ is (C₁-C₄)alkyl;
R₅ is hydrogen or a radical selected from the group consisting of: —C(O)(C₁-C₄)alkyl; and —SO₂—CH₃;

### R₃ is —CH₂—R₁₅;

R₁₅ is a C-radical which is a known ring system with 2 rings totally fused, wherein each one of the rings:
has 5-6 members, each member independently selected from C, O, CH, and CH₂,
is saturated, partially unsaturated or aromatic, and
   being each ring forming part of the ring system optionally substituted by one one halogen radical.

According to the present invention, the terms “alkyl”, “alkenyl” and “alkynyl” embraces both straight and branched chains.

According to the present invention when the ring system is formed by “isolated” rings means that the ring system is formed by two, three or four rings and said rings are bound via a bond from the atom of one ring to the atom of the other ring.

The term “isolated” also embraces the embodiment in which the ring system has only one ring. Illustrative non-limitative examples of known ring systems consisting of one ring are those derived from: cyclopropyl, cyclobutyl, cyclopentyl, cyclohexyl, cycloheptyl, cyclopropenyl, cyclobutenyl, cyclopentenyl, phenyl, cycloheptenyl, aziridinyl, oxirenyl, thiiranyl, azetidinyl, oxetanyl, pyrrolyl, furanyl, thiophenyl, pyrazolyl, imidazolyl, triazolyl, tetrazolyl, oxazolyl, isoxazolyl, thiazolyl, isothiazolyl, dithiolanyl, pyridinyl, pyranyl, pyridazinyl, pyrimidinyl, pyrazinyl, piperazinyl, oxazinyl, thiazinyl, dithianyl, and dioxanyl.

According to the present invention when the ring system has rings “totally fused”, means that the ring system is formed by two, three or four rings in which two or more atoms are common to two adjoining rings. Illustrative non-limitative examples of known ring systems consisting of two rings totally fused, are those derived from benzofuran, isobenzofuran, indole, isoindole, indolizine, indoline, isoindoline, purine (e.g., adenine, guanine), benzimidazole, indazole, benzoxazole, benzisoxazole, benzodioxole, benzofurazan, benzotriazole, benzothiofuran, benzothiazole, benzothiadiazole, heterocyclic chromene, isochromene, chroman, isochroman, benzodioxan, quinoline, isoquinoline, quinolizine, benzoxazine, benzodiazine, pyridopyridine, quinoxaline, quinazoline, cinnoline, phthalazine, naphthyridine, pteridine. Illustrative non-limitative examples of known ring systems consisting of 3 fused rings are carbazole, dibenzofuran, dibenzothiophene, carboline, perimidine, pyridoindole, acridine, xanthene, thioxanthene, oxanthrene, phenoxathiin, phenazine, phenoxazine, phenothiazine, thianthrene, phenanthridine, phenanthroline, phenazine.

According to the present invention when the ring system is “partially fused” it means that the ring system is formed by three or four rings, being at least two of said rings totally fused (i.e. two or more atoms being common to the two adjoining rings) and the remaining ring(s) being bound via a bond from the atom of one ring to the atom of one of the fused rings.

In still yet another embodiment of the first aspect of the invention the compound is selected from the group consisting of:
5-amino-1-(5-amino-pentyl)-2-(6-iodo-benzo[1,3]dioxol-5-ylsulfanyl)-1H-imidazole-4-carboxamide;
5-amino-1-butyl-2-(6-iodo-benzo[1,3]dioxol-5-ylsulfanyl)-1H-imidazole-4-carboxamide;
5-amino-2-(benzo[d][1,3]dioxol-5-ylthio)-1-butyl-1H-imidazole-4-carboxamide;
5-amino-1-(5-aminopentyl)-2-(benzo[d][1,3]dioxol-5-ylthio)-1H-imidazole-4-carboxamide;
5-acetamido-2-(benzo[d][1,3]dioxol-5-ylthio)-1-butyl-1H-imidazole-4-carboxamide;
2-(4-(trifluoromethyl)phenylthio)-5-amino-1-butyl-1H-imidazole-4-carboxamide;
5-amino-1-butyl-2-(2,5-dimethoxy-phenylsulfanyl)-1H-imidazole-4-carboxamide;
5-acetylamino-1-butyl-2-(6-iodo-benzo[1,3]dioxol-5-ylsulfanyl)-1H-imidazole-4-carboxamide; -5-amino-2-(6-iodo-benzo[1,3]dioxol-5-ylsulfanyl)-1-(3-isopropylamino-propyl)-1H-imidazole-4-carboxamide.
5-amino-2-(benzo[d][1,3]dioxol-5-ylthio)-1-butyl-1H-imidazole-4-carboxamide;
5-amino-1-butyl-2-(7-chloro-benzothiazol-2-ylsulfanyl)-1H-imidazole-4-carboxamide;
5-amino-1-butyl-2-(4-phenyl-thiazol-2-ylsulfanyl)-1H-imidazole-4-carboxamide;
5-amino-2-(7-chloro-benzothiazol-2-ylsulfanyl)-1-methyl-1H-imidazole-4-carboxamide;
5-amino-2-(7-chloro-benzothiazol-2-ylsulfanyl)-1-(3-isopropylamino-propyl)-1H-imidazole-4-carboxamide;
5-amino-1-(3-isopropylamino-propyl)-2-(1-isopropyl-1H-benzoimidazol-2-ylsulfanyl)-1H-imidazole-4-carboxamide;
5-amino-2-(3-chloro-5-trifluoromethyl-pyridin-2-ylsulfanyl)-1-(3-isopropylamino-propyl)-1H-imidazole-4-carboxamide;
5-amino-1-(3-isopropylamino-propyl)-2-(naphthalen-2-ylsulfanyl)-1H-imidazole-4-carboxamide;
5-amino-1-(3-isopropylamino-propyl)-2-(4-phenyl-thiazol-2-ylsulfanyl)-1H-imidazole-4-carboxamide;
5-(cyclopropanecarbonyl-amino)-2-(6-iodo-benzo[1,3]dioxol-5-ylsulfanyl)-1-(3-isopropylamino-propyl)-1H-imidazole-4-carboxamide;
5-acetylamino-2-(7-chloro-benzothiazol-2-ylsulfanyl)-1-(3-isopropylamino-propyl)-1H-imidazole-4-carboxamide;
5-acetylamino-2-(6-iodo-benzo[1,3]dioxol-5-ylsulfanyl)-1-(3-isopropylamino-propyl)-1H-imidazole-4-carboxamide;
2-(6-Iodo-benzo[1,3]dioxol-5-ylsulfanyl)-1-methyl-5-phenylacetylamino-1H-imidazole-4-carboxamide;
2-(5-iodobenzo[d][1,3]dioxol-6-ylthio)-1-methyl-5-(phenethylamino)-1H-imidazole-4-carboxamide;
5-amino-2-[(6-bromo-1,3-benzodioxol-5-yl)thio]-1-methyl-1H-imidazole-4-carboxamide;
5-amino-2-[(6-isopropyl-1,3-benzodioxol-5-yl)thio]-1-methyl-1H-imidazole-4-carboxamide;
5-amino-1-methyl-2-[(6-vinyl-1,3-benzodioxol-5-yl)thio]-1H-imidazole-4-carboxamide;
5-amino-2-[(6-ethoxy-1,3-benzodioxol-5-yl)thio]-1-methyl-1H-imidazole-4-carboxamide;
5-amino-1-methyl-2-{[6-(methylthio)-1,3-benzodioxol-5-yl]thio}-1H-imidazole-4-carboxamide;
5-amino-1-methyl-2-{[6-(methylsulfonyl)-1,3-benzodioxol-5-yl]thio}-1H-imidazole-4-carboxamide;
5-amino-2-[(6-iodo-1,3-benzodioxol-5-yl)thio]-1-methyl-1H-imidazole-4-carboxamide;
5-amino-2-[(6-bromo-1,3-benzodioxol-5-yl)thio]-1-(3-morpholin-4-ylpropyl)-1H-imidazole-4-carboxamide;
5-amino-2-[(6-iodo-1,3-benzodioxol-5-yl)thio]-1-(3-morpholin-4-ylpropyl)-1H-imidazole-4-carboxamide;
5-amino-2-[(6-iodo-1,3-benzodioxol-5-yl)thio]-1-[3-(diethylamino)propyl]-1H-imidazole-4-carboxamide;
5-amino-2-[(6-bromo-1,3-benzodioxol-5-yl)thio]-1-[3-(diethylamino)propyl]-1H-imidazole-4-carboxamide;
2-[(6-bromo-1,3-benzodioxol-5-yl)thio]-1-methyl-5-[(methylsulfonyl)amino]-H-imidazole-4-carboxamide;
5-(acetylamino)-2-[(6-bromo-1,3-benzodioxol-5-yl)thio]-1-methyl-1H-imidazole-4-carboxamide;
4-amino-1-(6-bromo-benzo[1,3]dioxol-5-ylmethyl)-5-methyl-1H-pyrazole-3-carboxamide;
4-(acetylamino)-1-[(6-bromo-1,3-benzodioxol-5-yl)methyl]-5-methyl-1H-pyrazole-3-carboxamide;
1-[(6-bromo-1,3-benzodioxol-5-yl)methyl]-5-methyl-4-(propionylamino)-1H-pyrazole-3-carboxamide;
1-[(6-bromo-1,3-benzodioxol-5-yl)methyl]-5-methyl-4-[(methylsulfonyl)amino]-1H-pyrazole-3-carboxamide;
4-amino-1-[(6-iodo-1,3-benzodioxol-5-yl)methyl]-5-methyl-1H-pyrazole-3-carboxamide;
5-amino-2-[(6-bromo-2,3-dihydro-1-benzofuran-5-yl)thio]-1-methyl-1H-imidazole-4-carboxamide;
5-amino-2-{[6-(methylthio)-1,3-benzodioxol-5-yl]thio}-1-[3-(diethylamino) propyl]-1H-imidazole-4-carboxamide.
5-amino-2-{[6-(ethylthio)-1,3-benzodioxol-5-yl]thio}-1-[3-(diethylamino) propyl]-1H-imidazole-4-carboxamide; and
5-amino-2-{[6-(methylthio)-2,3-dihydro-1-benzofuran-5-yl]thio}1-methyl-1H-imidazole-4-carboxamide

The compounds of the present invention can be obtained as it is illustrated in Scheme I:

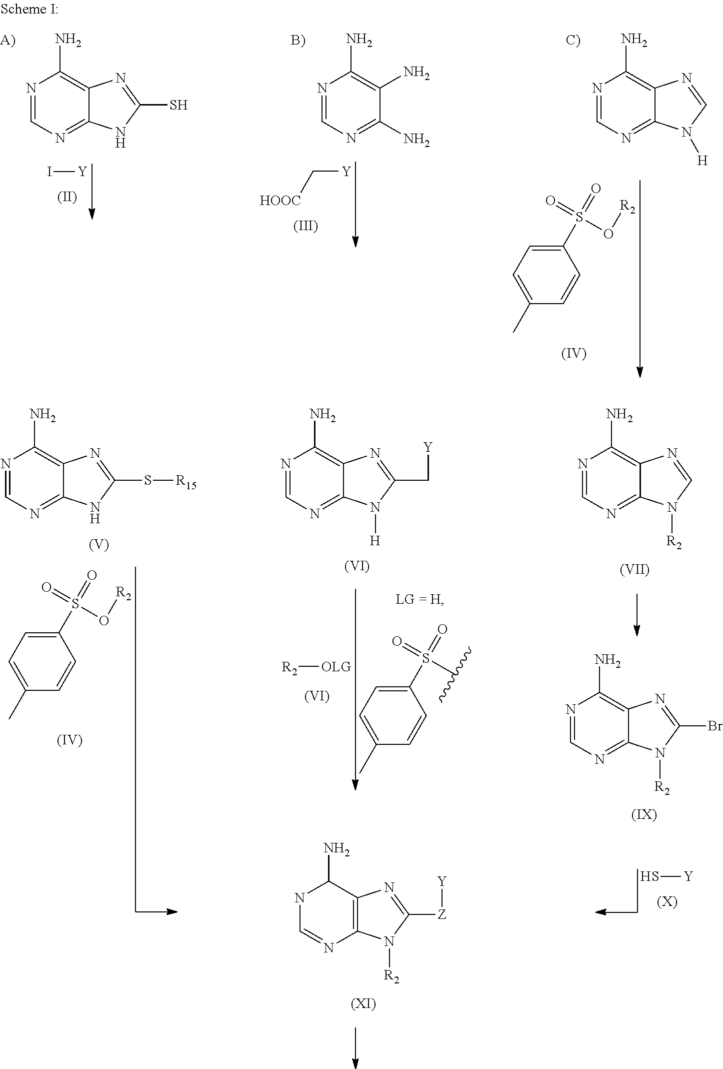

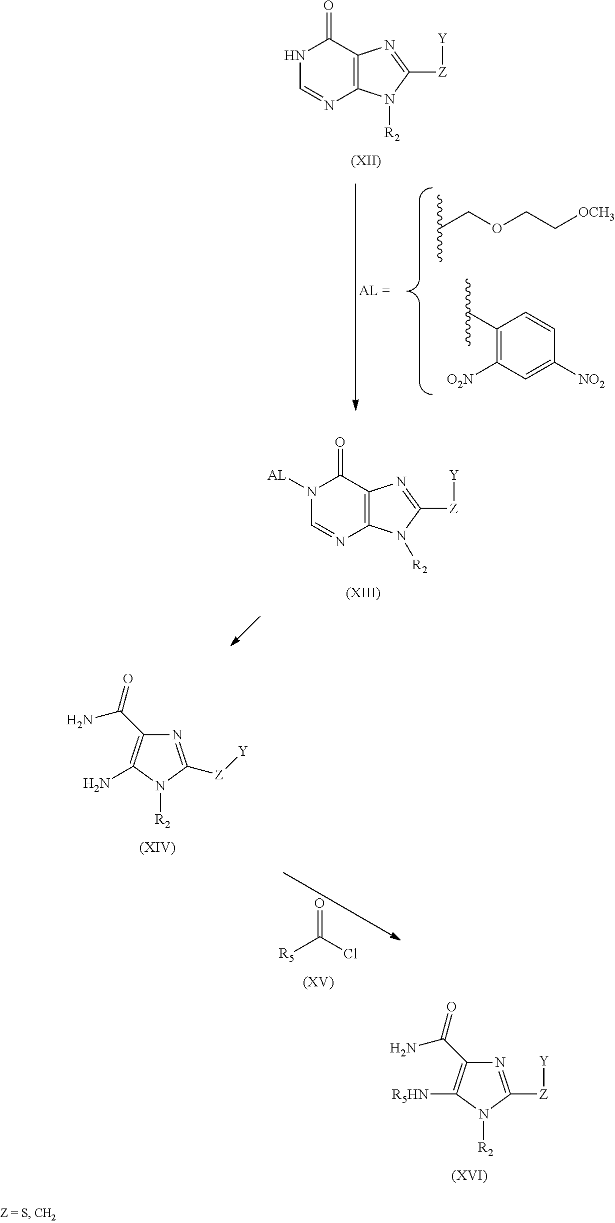

(In compounds of general formula (XVI): Z is —S— when Y is R₁₄, and Z is —CH₂— when Y is R₁₅),

Briefly, the compounds of formula (XVI) where Z═S, Y═R₁₄ and a is nitrogen, and b, c, d are carbon atoms can be prepared according to the processes summarized in Schemes I-A, and II-C. The process described in scheme I-A involves a coupling reaction of 6-amino-9H-purine-8-thiol (Chiosis G. et al., “Identification of Potent Water Soluble Purine-Scaffold Inhibitors of the Heat Shock Protein 90”, J. Med. Chem., 2006, vol. 49, no. 1, p. 381) with a compound of formula (II), followed by the N-alkylation of compound (V) with the toluenesulfonyloxy derivative (IV) (can be obtained by using standards processes of organic chemistry) to give compound of formula (XI) with Z═S. Also, the process described in Scheme 1-C gives the same compound of formula (XI) with Z═S, starting for 9H-purin-6-ylamine. N-alkylation of 9H-purin-6-ylamine with toluenesulfonyloxy derivative (IV)) (can be obtained by using standards processes of organic chemistry) gives the compound of formula (VII), which followed by the bromination reaction gives the compound of formula (IX). Then coupling of compound of formula (IX) with compound of formula (X) gives the compound of formula (XI) with Z═S.

Compounds of formula (XVI) where Z═—CH₂—, Y═R₁₅ and a is nitrogen, and b, c and d are carbon atoms can be prepared according to the process summarized in Scheme I-B. The process involves the acylation of 4,5,6-triaminopyrimide with compounds of formula (III) and cyclization of the resulting intermediate in basic conditions. Then, N-alkylation of compounds of formula (VI) with compounds of formula (VIII) gives compounds of formula (XI) with Z═—CH₂—.

At this point, compounds of formula (XI) with Z═S or —CH₂— follow the same synthetic steps to achieve the final compounds of formula (XVI).

Oxidation-deamination of compounds of formula (XI) gives compounds of formula (XII), then, alquilation with either 2-methoxy-ethoxymethyl or 2,4-dinitrophenyl reagent derivatives gives compounds of formula (XIII). Ring opening of compounds of formula (XIII) with ethylenediamine gives compounds of formula (XIV) and those can be acylated with the appropriate acylchloride reagent (of formula (XV) to give the final compounds of formula (XVI), with Z being S or —CH₂—, respectively.

Optionally, compounds of formula where Z═S, Y═R₁₄ and a is nitrogen, and b, c and d are carbon atoms can be prepared according to the processes summarized in Schemes (II) and (III).

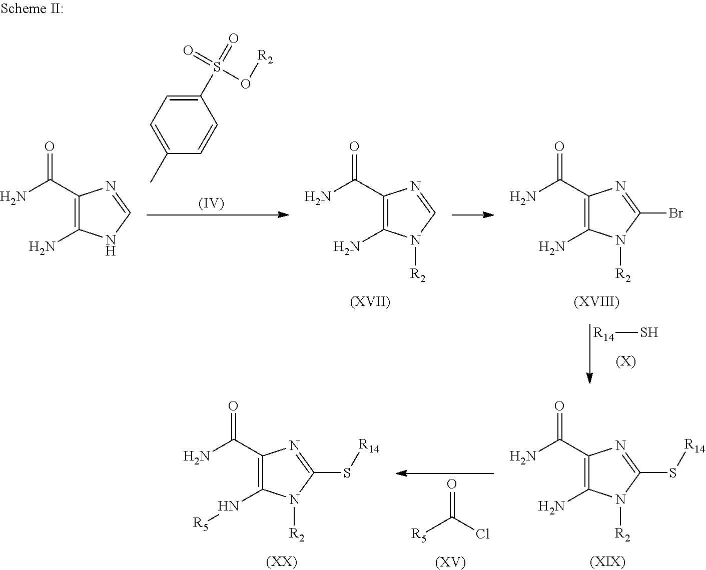

The process described in scheme II involves an N-alkylation reaction of 5-aminoimidazole-4-carboxamide (AICA) with the toluenesulfonyloxy derivative (IV) to give the compound of formula (XVII), followed by the bromination reaction giving the compound of formula (XVIII). The coupling of compound of formula (XVIII) with the compound of formula (X) gives the compound of formula (XIX). When R₂ is a protected carbonated chain an additional deprotection is required before or after the acylation step depends on the final compound to synthesize. Acylation reaction of the compound of formula (XIX) with the appropriate acyl chloride reagent (of formula XV) gives the final compounds of formula (XX).

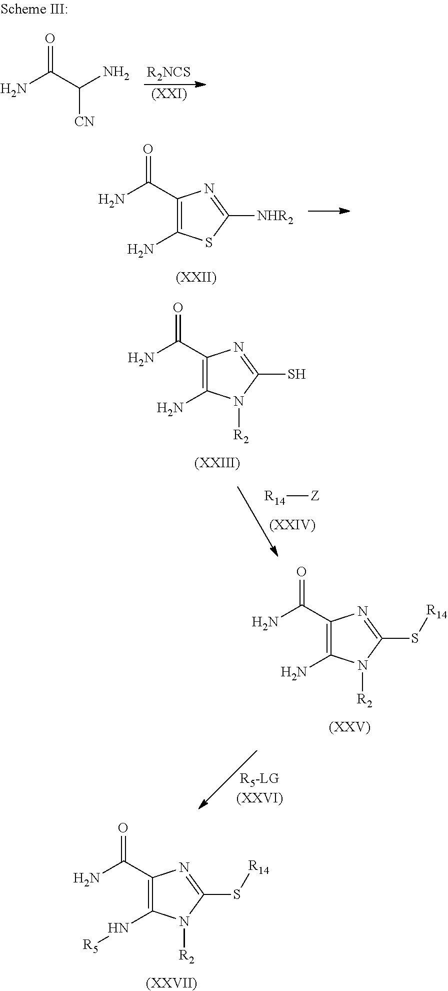

The process described in Scheme III involves the isothiocyanate condensation of the isothiocyanate of formula (XXI) with 2-amino-2-cyanoacetamide, followed by rearrangement in basic conditions to give the compound of formula (XXIII). The coupling of compound of formula (XXIII) with the compound of formula (XXIV), wherein Z represents bromine or iodine, gives the compound of formula (XXV). Different acylation or reductive amination reactions of the compound of formula (XXV) with the appropriate reagent (of formula XXVI, wherein LG represents different sulfonyl or acid chloride, or aldehyde derivatives) gives the final compounds of formula (XXVII).

Compounds of formula where Z═S, Y═R₁₄ and b is nitrogen, and a, c and d are carbon atoms can be prepared according to the process summarized in Scheme (IV).

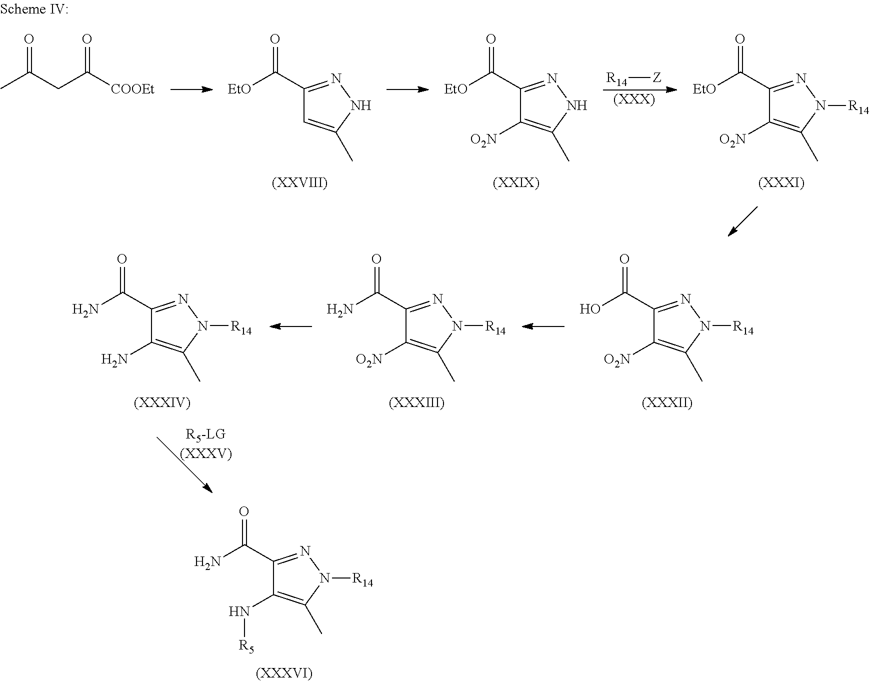

The process described in Scheme IV involves the cyclation reaction of ethyl 2,4-dioxopentanoate with hydrazine monohydrate to give the compound of formula (XXVIII), followed by the nitration reaction giving the compound of formula (XXIX). N-Alkylation of compound of formula (XXIX) with the compound of formula (XXX) gives the compound of formula (XXXI). Hydrolysis of ester residue of the compound of formula (XXXI) gives the compound of formula (XXXII). This compound of formula (XXXII) was transformed into amide by reaction with thionyl chloride and ammonia, sequentially, to give the compound of formula (XXXIII). Reduction of the nitro residue of compound of formula (XXXIII) gives the compound of formula (XXXIV). The acylation reaction of the compound of formula (XXXIV) with the appropriate reagent of formula XXXV, wherein LG represents different sulfonyl or acid chloride derivatives gives the final compounds of formula (XXXVI).

The pharmaceutical composition (e.g. formulation) may comprise a therapeutically effective amount of the compound of formula (I), as defined above, together with one or more pharmaceutically acceptable excipients or carriers such as adjuvants, diluents, fillers, buffers, stabilisers, preservatives, lubricants.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The term “therapeutically-effective amount,” as used herein, pertains to that amount of an active compound, or a material, composition or dosage form comprising an active compound, which is effective for producing some desired therapeutic effect.

As it is shown below, the compounds of the present invention are Hsp90 inhibitors, being useful in the treatment of a disease mediated by Hsp90.

In a preferred embodiment it is provided the use of a compound of formula (I) as defined in the preferred embodiments pointed out above, for the first aspect of the invention for the manufacture of a medicament for the treatment of a disease mediated by a heat shock protein 90. This aspect of the invention can also be formulated as compound of formula (I) as defined in the fourth aspect of the invention for use in the treatment of a disease mediated by a heat shock protein 90.

The term “a disease mediated by Hsp90,” as used herein pertains to a condition in which Hsp90 and/or the action of Hsp90 is important or necessary, e.g., for the onset, progress, expression, etc. of that condition. Examples of conditions mediated by Hsp90 include, but are not limited to, a condition characterized by Hsp90 action upon a client protein which drives that condition; a condition characterized by one or more client proteins which are acted upon by Hsp90; a condition driven by one or more proteins, which proteins are Hsp90 client proteins, and which proteins could not drive the condition in the absence of action (e.g., chaperoning) by Hsp90; a condition driven by one or more proteins, which proteins are Hsp90 client proteins, and the action (e.g., chaperoning) by Hsp90 in order to drive the condition.

Examples of such conditions include, but are not limited to: cancer; immunosuppressive applications such auto-immune disease; arthritis; prion diseases (e.g., Creutzfeld Jacob Disease (CJD), variant CJD); other diseases associated with defects in protein folding and aggregation (e.g., Alzheimer's disease, Huntingdon's disease) and diseases caused by virus. (cf. Pacey, S. et al., supra) (cf., Blagg, B. S. J and Kerr, T. D., “Hsp90 inhibitors: small molecules that transform the Hsp90 protein folding machinery into a catalyst for protein degradation”, Medicinal Research Review, 2005, 1-29) (cf., Kamal A. et al, “Therapeutic and diagnostic implications of Hsp90 activation”, TRENDS in Molecular Medicine, 2004, vol 10, 283-290).

For example, many oncoproteins are Hsp90 client proteins. In the absence of the chaperoning action of Hsp90, these proteins are degraded, for example, by ubiquitin dependent proteasome degradation. Similarly, the LCK protein, characteristic of many autoimmune diseases, is also an Hsp90 client protein. In the absence of the chaperoning action of Hsp90, LCK levels are reduced. Thus, in one embodiment, the present invention provides active compounds which are anticancer agents. The term “anticancer agent” as used herein, pertains to a compound which treats a cancer (i.e., a compound which is useful in the treatment of a cancer). The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a turnout from its origin), the inhibition of invasion (the spread of turnout cells into neighbouring normal structures), or the promotion of apoptosis (programmed cell death).

Thus, the present invention also provides active compounds which are antiproliferative agents. The term “antiproliferative agent” as used herein, refers to a compound which treats a proliferative condition (i.e., a compound which is useful in the treatment of a proliferative condition).

The terms “cell proliferation,” “proliferative condition,” “proliferative disorder,” and “proliferative disease,” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, benign, pre-malignant, and malignant cellular proliferation, including but not limited to, neoplasms and tumors (e.g., histocytoma, glioma, astrocyoma, osteoma), cancers (e.g., lung cancer, small cell lung cancer, gastrointestinal cancer, bowel cancer, colon cancer, breast carcinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), leukemias, psoriasis, bone diseases, fibroproliferative disorders (e.g., of connective tissues), and atherosclerosis.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included. The term “treatment” includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.)); surgery; radiation therapy; and gene therapy.

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. Furthermore, the present invention covers all possible combinations of particular and preferred groups described hereinabove.

The following examples and drawings are provided by way of illustration, and are not intended to be limiting of the present invention.

### EXAMPLES

### Example 1

### Preparation of toluene-4-sulfonic acid 6-(1,3-dioxo-1,3-dihydro-isoindol-2-yl)-pentyl ester

1,8-naphthalic anhydride (2.8 g, 19 mmol) was added to a solution 6-Amino-pentan-1-ol (2 g, 19 mmol) in absolute ethanol (140 mL). The reaction mixture was heated at reflux overnight. Removal of the solvent and purification by chromatography on alumina (CH₂Cl₂/MeOH, 50:1) yielded 2-(6-hydroxy-pentyl)-isoindole-1,3-dione (Hamilton, R., “A convenient synthesis of N-protected diphenyl phosphonate ester analogues of ornithine, lysine and homolysine”, Tetrahedron Letters, 1993, 34(17), 2847-50) (4.5 g, 100%) as a foam. ¹H-NMR [CDCl₃, δ, ppm]: 8.62 (m, 2H), 8.22 (m, 2H), 3.69 (m, 2H, CH₂O), 3.58 (m, 2H, CH₂NO), 1.72 (m, 2H, CH₂), 1.41 (m, 4H, CH₂).

A solution of 2-(6-hydroxy-pentyl)-isoindole-1,3-dione (4.5 g, 19 mmol), p-toluensulfonyl chloride (4.1 g, 21 mmol), and pyridine (1.8 mL, 22 mmol) in 80 mL of CH₂Cl₂ was stirred at room temperature for 16 h. Removal of the solvent and purification by chromatography on silica (hexane/CH₂Cl₂/AcOEt, 5:4:1) yielded toluene-4-sulfonic acid 6-(1,3-dioxo-1,3-dihydro-isoindol-2-yl)-pentyl ester (2.7 g, 36%) as a viscous oil. ¹H-NMR [CDCl₃, δ, ppm]:), 8.62 (m, 2H), 8.22 (m, 2H), 7.79 (d, J=5 Hz, 2H), 7.36 (d, J=5 Hz, 2H, 4.02 (m, 2H, CH₂O), 3.58 (m, 2H, CH₂NO), 2.46 (s, 3H, CH₃), 1.72 (m, 2H, CH₂), 1.41 (m, 4H, CH₂).

### Example 2

### Preparation of 2-{6-[6-amino-8-(6-iodo-benzo[1,3]dioxol-5-ylsulfanyl)-purin-9-yl]-pentyl}-isoindole-1,3-dione

A solution of 8-(6-iodo-benzo[1,3]dioxol-5-ylsulfanyl)-9H-purin-6-ylamine previously prepared (cf. Chiosis G. et al. “Identification of Potent Water Soluble Purine-Scaffold Inhibitors of the Heat Shock Protein 90”, J. Med. Chem., 2006, vol. 49, no. 1, p. 381) (0.5 g, 1.2 mmol), toluene-4-sulfonic acid 6-(1,3-dioxo-1,3-dihydro-isoindol-2-yl)-pentyl ester (0.7 g, 1.8 mmol), and Cs₂CO₃ (0.4 g, 1.2 mmol) in 36 mL of DMF was stirred at 80° C. for 16 h. Removal of the solvent and purification by chromatography on silica (CHCl₃/AcOEt/MeOH, 5:4:1) yielded 2-{6-[6-Amino-8-(6-iodo-benzo[1,3]dioxol-5-ylsulfanyl)-purin-9-A-pentyl}-isoindole-1,3-dione (0.2 g, 27%) as a viscous oil. ¹H-NMR, CDCl₃ δ, ppm]: 8.32 (s, 1H), 7.81 (m, 2H), 7.68 (m, 2H), 7.49 (s, 1H), 7.30 (s, 1H), 5.99 (s, 2H, OCH₂O), 4.16 (m, 2H, CH₂N), 3.64 (m, 2H, CH₂NO), 1.72 (m, 2H, CH₂), 1.41 (m, 4H, CH₂).

### Example 3

### Preparation of 2-{5-[8-(6-iodo-benzo[1,3]dioxol-5-ylsulfanyl)-6-oxo-1,6-dihydro-purin-9-yl]-pentyl}-isoindole-1,3-dione

A solution of 2-{6-[6-Amino-8-(6-iodo-benzo[1,3]dioxol-5-ylsulfanyl)-purin-9-A-pentyl}-isoindole-1,3-dione (0.56, 0.89 mmol) in HOAc (16 mL) was stirred at room temperature, and then NaNO₂ (0.61 g, 8.84 mmol) in H₂O (5 mL) was added. Then the reaction mixture was stirred at room temperature for 16 h. The solvent was removed and water was added to the residue. The aqueous phase was extracted with AcOEt. The organic phase was washed (NaHCO₃, brine) and dried (MgSO₄). Removal of the solvent and purification by chromatography on silica (CHCl₃/AcOEt/MeOH, 5:4:1) yielded 2-{5-[8-(6-Iodo-benzo[1,3]dioxol-5-ylsulfanyl)-6-oxo-1,6-dihydro-purin-9-A-pentyl}-isoindole-1,3-dione (0.32 g, 57%) as a foam. ¹H-NMR [CDCl₃, δ, ppm]: 8.01 (s, 1H), 7.81 (m, 2H), 7.68 (m, 2H), 7.49 (s, 1H), 7.30 (s, 1H), 5.99 (s, 2H, OCH₂O), 4.21 (m, 2H, CH₂N), 3.65 (m, 2H, CH₂NO), 1.72 (m, 4H, CH₂), 1.39 (m, 2H, CH₂).

### Example 4

### Preparation of 2-{5-[8-(6-iodo-benzo[1,3]dioxol-5-ylsulfanyl)-1-(2-methoxy-ethoxymethyl)-6-oxo-1,6-dihydro-purin-9-yl]-pentyl}-isoindole-1,3-dione

To a solution of 2-{5-[8-(6-Iodo-benzo[1,3]dioxol-5-ylsulfanyl)-6-oxo-1,6-dihydro-purin-9-yl]-pentyl}-isoindole-1,3-dione (0.32 g, 0.5 mmol) in CH₂Cl₂ (20 mL) was added N,N-diisopropylethylamine (0.1 mL, 0.77 mmol) at room temperature, and 2-methoxyethoxymethyl chloride (0.1 mL, 0.8 mmol) was added drop wise. The mixture was stirred at room temperature for 16 h, then quenched by pouring over water and the aqueous phase was extracted with CHCl₃. The organic phase was washed (NaHCO₃, brine) and dried (MgSO₄). Removal of the solvent and purification by chromatography on silica (CHCl₃/AcOEt/MeOH, 5:4:1) yielded 2-{5-[8-(6-Iodo-benzo[1,3]dioxol-5-ylsulfanyl)-1-(2-methoxy-ethoxymethyl)-6-oxo-1,6-dihydro-purin-9-yl]-pentyl}-isoindole-1,3-dione (0.22 g, 62%) as a viscous oil. ¹H-NMR CDCl₃, δ, ppm]: 8.08 (s, 1H), 7.81 (m, 2H), 7.68 (m, 2H), 7.49 (s, 1H), 7.30 (s, 1H), 5.99 (s, 2H, OCH₂O), 5.52 (s, 2H, N(CH₂)O), 4.15 (m, 2H, CH₂N), 3.80-3.79 (m, 4H, OCH₂CH₂O), 3.65 (m, 2H, CH₂NO), 3.33 (s, 3H, OCH₃), 1.72 (m, 4H, CH₂), 1.39 (m, 2H, CH₂).

### Example 5

### Preparation of 5-amino-1-[5-(1,3-dioxo-1,3-dihydro-isoindol-2-yl)-pentyl ]-2-(6-iodo-benzo[1,3]dioxol-5-ylsulfanyl)-1H-imidazole-4-carboxamide

2-{5-[8-(6-Iodo-benzo[1,3]dioxol-5-ylsulfanyl)-1-(2-methoxy-ethoxymethyl)-6-oxo-1,6-dihydro-purin-9-yl]-pentyl}-isoindole-1,3-dione (0.22 g, 0.31 mmol) was suspended with 0.2 N NaOH aqueous solution (6 mL) and heated under reflux for 1 h. After cooling, the reaction mixture was neutralized with 3N HCl aqueous solution, and concentrated in vacuum. Purification of the crude product by flash chromatography (SiO₂, CHCl₃/MeOH, 7:1) yielded 5-Amino-1-[5-(1,3-dioxo-1,3-dihydro-isoindol-2-yl)-pentyl]-2-(6-iodo-benzo[1,3]dioxol-5-ylsulfanyl)-1H-imidazole-4-carbox amide (80 mg, 42%) as a foam. ¹H-NMR [CDCl₃, δ, ppm]: 7.81 (m, 2H), 7.68 (m, 2H), 7.49 (s, 1H), 7.30 (s, 1H), 5.99 (s, 2H, OCH₂O), 3.75 (m, 2H, CH₂N), 3.65 (m, 2H, CH₂NO), 1.72 (m, 4H, CH₂), 1.39 (m, 2H, CH₂).

### Example 6

### Preparation of 5-amino-1-(5-amino-pentyl)-2-(6-iodo-benzo[1,3]dioxol-5-ylsulfanyl)-1H-imidazole-4-carboxamide

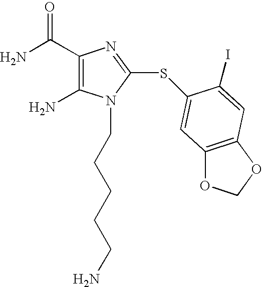

A 33% solution of methylamine in absolute ethanol (14 mL) is added to a stirred suspension of 5-Amino-1-[5-(1,3-dioxo-1,3-dihydro-isoindol-2-yl)-pentyl]-2-(6-iodo-benzo[1,3]dioxol-5-ylsulfanyl)-1H-imidazole-4-carboxylic acid amide (80 mg, 0.13 mmol) in EtOH (10 mL) at room temperature. After 5 min, the clear solution obtained is refluxed for 16 h. and the mixture is cooled at room temperature. Removal of the solvent and purification by chromatography on silica (CHCl₃/MeOH/NH₄OH, 5:1:0.5) yielded 5-amino-1-(5-amino-pentyl)-2-(6-iodo-benzo[1,3]dioxol-5-ylsulfanyl)-1H-imidazole-4-carboxamide (6.4 mg, 10%) as a foam. ¹H-NMR [MeOD, δ, ppm]: 7.17 (s, 1H), 6.45 (s, 1H), 5.92 (s, 2H, OCH₂O), 3.78 (m, 2H, CH₂N), 2.85 (m, 2H, CH₂NH₂), 1.51 (m, 4H, CH₂), 1.39 (m, 2H, CH₂). MS (EI, m/z) 490 (M⁺+1).

### Example 7

### Preparation of 9-butyl-8-(6-iodo-benzo[1,3]dioxol-5-ylsulfanyl)-9H-purin-6-ylamine

A solution of 8-(6-iodo-benzo[1,3]dioxol-5-ylsulfanyl)-9H-purin-6-ylamine previously prepared (cf. Chiosis G. et al., “Identification of Potent Water Soluble Purine-Scaffold Inhibitors of the Heat Shock Protein 90”, J. Med. Chem. 2006, vol. 49, no. 1, p. 381 (0.3 g, 0.73 mmol), toluene-4-sulfonic acid butyl ester previously prepared (cf. Sekera, V. et al., “Higher alkyl sulfonates”, J. Am. Chem. Soc., 1993, vol. 55, p. 345 (0.7 g, 1.8 mmol), and Cs₂CO₃ (0.4 g, 1.7 mmol) in 7 mL of DMF was stirred at 80° C. for 16 h. Removal of the solvent and purification by chromatography on silica (CHCl₃/AcOEt/MeOH, 5:4:1) yielded 9-Butyl-8-(6-iodo-benzo[1,3]dioxol-5-ylsulfanyl)-9H-purin-6-ylamine (0.17 g, 49%) as a viscous oil. ¹H-NMR [CDCl₃, δ, ppm]: 8.32 (s, 1H), 7.29 (s, 1H), 7.19 (s, 1H), 5.97 (s, 2H, OCH₂O), 4.20 (m, 2H, CH₂N), 1.76 (m, 2H, CH₂), 1.40 (m, 2H, CH₂), 1.37 (m, 3H, CH₃).

### Example 8

### Preparation of 9-butyl-8-(6-iodo-benzo[1,3]dioxol-5-ylsulfanyl)-1,9-dihydro-purin-6-one

A solution of 9-butyl-8-(6-iodo-benzo[1,3]dioxol-5-ylsulfanyl)-9H-purin-6-ylamine (0.16, 0.35 mmol) in HOAc (5 mL) was stirred at room temperature, and then NaNO₂ (0.24 g, 3.48 mmol) in H₂O (2 mL) was added. Then the reaction mixture was stirred at room temperature for 16 h. The solvent was removed and water was added to the residue. The aqueous phase was extracted with AcOEt. The organic phase was washed (NaHCO₃, brine) and dried (MgSO₄). Removal of the solvent and purification by chromatography on silica (CHCl₃/AcOEt/MeOH, 5:4:1) yielded 9-Butyl-8-(6-iodo-benzo[1,3]dioxol-5-ylsulfanyl)-1,9-dihydro-purin-6-one (83 mg, 50%) as a foam. ¹H-NMR [CDCl₃, δ, ppm]: 7.92 (s, 1H), 7.29 (s, 1H), 7.19 (s, 1H), 5.97 (s, 2H, OCH₂O), 4.24 (m, 2H, CH₂N), 1.76 (m, 2H, CH₂), 1.40 (m, 2H, CH₂), 1.37 (m, 3H, CH₃).

### Example 9

### Preparation of 9-butyl-8-(6-iodo-benzo[1,3]dioxol-5-ylsulfanyl)-1-(2-methoxy-ethoxymethyl)-1,9-dihydro-purin-6-one

To a solution of 9-butyl-8-(6-iodo-benzo[1,3]dioxol-5-ylsulfanyl)-1,9-dihydro-purin-6-one (83 mg, 0.17 mmol) in CH₂Cl₂ (5 mL) was added N,N-diisopropylethylamine (0.1 mL, 0.77 mmol) at room temperature, and 2-methoxyethoxymethyl chloride (0.1 mL, 0.8 mmol) was added drop wise. The mixture was stirred at room temperature for 16 h, then quenched by pouring over water and the aqueous phase was extracted with CHCl₃. The organic phase was washed (NaHCO₃, brine) and dried (MgSO₄). Removal of the solvent and purification by chromatography on silica (CHCl₃/AcOEt/MeOH, 5:4:1) yielded 9-Butyl-8-(6-iodo-benzo[1,3]dioxol-5-ylsulfanyl)-1-(2-methoxy-ethoxymethyl)-1,9-dihydro-purin-6-one (59 mg, 60%) as a viscous oil. ¹H-NMR [CDCl₃, δ, ppm]: 8.08 (s, 1H), 7.26 (s, 1H), 6.95 (s, 1H), 5.94 (s, 2H, OCH₂O), 5.53 (s, 2H, N(CH₂)O), 4.15 (m, 2H, CH₂N), 3.80-3.79 (m, 4H, OCH₂CH₂O), 3.36 (s, 3H, OCH₃), 1.76 (m, 2H, CH₂), 1.40 (m, 2H, CH₂), 1.37 (m, 3H, CH₃).

### Example 10

### Preparation of 5-amino-1-butyl-2-(6-iodo-benzo[1,3]dioxol-5-ylsulfanyl)-1H-imidazole-4-carboxamide

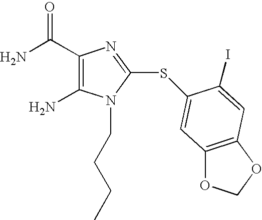

9-butyl-8-(6-iodo-benzo[1,3]dioxol-5-ylsulfanyl)-1-(2-methoxy-ethoxymethyl)-1,9-dihydro-purin-6-one (39 mg, 69 μmol) was suspended with 0.2 N NaOH aqueous solution (2 mL) and heated under reflux for 1 h. After cooling, the reaction mixture was neutralized with 3N HCl aqueous solution, and concentrated in vacuum. Purification of the crude product by flash chromatography (SiO₂, CHCl₃/MeOH, 2%) yielded 5-amino-1-butyl-2-(6-iodo-benzo[1,3]dioxol-5-ylsulfanyl)-1H-imidazole-4-carboxamide (3 mg, 8%) as a foam. ¹H-NMR [MeOD, δ, ppm]: 7.21 (s, 1H), 6.49 (s, 1H), 5.95 (s, 2H, OCH₂O), 3.79 (m, 2H, CH₂N), 1.54 (m, 2H, CH₂), 1.37 (m, 2H, CH₂), 0.92 (m, 3H, CH₃). MS (EI, m/z) 461 (M⁺+1).

### Example 11

### Preparation of 8-(benzo[d][1,3]dioxol-5-ylthio)-9-butyl-9H-purin-6-amine

257 mg (0.75 mmol) of 8-(benzo[d][1,3]dioxol-5-ylthio)-9H-purin-6-amine previously prepared cf. Chiosis G. et al., “Identification of Potent Water Soluble Purine-Scaffold Inhibitors of the Heat Shock Protein 90”, J. Med. Chem., 2006, vol. 49, no. 1, p. 381) and 244 mg (0.75 mmol) of Cs₂CO₃ were suspended in distilled DMF (3 mL). Over this suspension, 257 mg (1.13 mmol) of butyl-4-methylbenzenesulfonate (cf. Sekera et al., “Higher alkyl sulfonates”, J. Am. Chem. Soc., 1993, vol. 55, p. 345) were also added and the reaction mixture was stirred at 80° C. for 20 h. Solvent was removed under reduced pressure and the crude was purified by Flash chromatography (CH₂Cl₂/EtOAc/MeOH, 100:100:5) to give 155 mg (0.45 mmol, 60%) of 8-(benzo[d][1,3]dioxol-5-ylthio)-9-butyl-9H-purin-6-amine as a white solid. ¹H-NMR [CDCl₃, δ, ppm]: 8.28 (s, 1H), 7.02 (dd, J=8.0, 1.5 Hz, 1H), 6.96 (d, J=1.5 Hz, 1H), 6.77 (d, J=8.0 Hz, 1H), 6.03 (br s, 2H), 5.95 (s, 2H, OCH₂O), 4.17 (t, J=7.5 Hz, 2H, CH₂N), 1.72 (m, 2H, CH₂), 1.35 (m, 2H, CH₂), 0.92 (t, J=7.5 Hz, 3H, CH₃).

### Example 12

### Preparation of 8-(benzo[d][1,3]dioxol-5-ylthio)-9-butyl-1H-purin-6 (9H)-one

8-(benzo[d][1,3]dioxol-5-ylthio)-9-butyl-9H-purin-6-amine (155 mg, 0.45 mmol) was dissolved in 6.5 mL of acetic acid. After 10 min stirring, a solution of 310 mg (4.5 mmol) of NaNO₂ in 2.5 mL of H₂O was injected over the yellow solution. The brown mixture was stirred for 20 h at 25° C. The solvent was removed under reduced pressure and the crude was taken up in a mixture of EtOAc/H₂O. The aqueous phase was extracted twice and the combined organic phases were washed (NaHCO₃, brine) and dried (MgSO₄). Removal of the solvent under reduced pressure and a further purification by Flash chromatography (CH₂Cl₂/EtOAc/MeOH, 100:100:3) yielded 90 mg (0.26 mmol, 58%) of 8-(benzo[d][1,3]dioxol-5-ylthio)-9-butyl-1H-purin-6 (9H)-one as a white-yellow solid. ¹H-NMR [CDCl₃, δ, ppm]: 8.04 (s, 1H), 7.09 (dd, J=8.0, 2.0 Hz, 1H), 7.05 (d, J=2.0 Hz, 1H), 6.78 (d, J=8.5 Hz, 1H), 6.00 (s, 2H, OCH₂O), 4.22 (t, J=7.5 Hz, 2H, CH₂N), 1.82 (br s, 2H), 1.76 (m, 2H, CH₂), 1.40 (m, 2H, CH₂), 0.97 (t, J=7.2 Hz, 3H, CH₃).

### Example 13

### Preparation of 8-(benzo[d][1,3]dioxol-5-ylthio)-9-butyl-1-(2,4-dinitrophenyl)-1H-purin-6 (9H)-one

8-(benzo[d][1,3]dioxol-5-ylthio)-9-butyl-1H-purin-6 (9H)-one (90 mg, 0.26 mmol), K₂CO₃ (90 mg, 0.65 mmol) and 1-chloro-2,4-dinitrobenzene (132 mg, 0.65 mmol) were dissolved in 4 mL of distilled DMF. The resulting suspension was stirred at 85° C. for 18 h. After removing the solvent under reduced pressure, the crude was purified by flash chromatography (CH₂Cl₂/EtOAc 1:1 with increasing amounts of methanol until 1%). 99 mg (0.20 mmol, 75%) of 8-(benzo[d][1,3]dioxol-5-ylthio)-9-butyl-1-(2,4-dinitrophenyl)-1H-purin-6 (9H)-one as a yellow solid were obtained. ¹H-NMR [CDCl₃, δ, ppm]: 8.97 (d, J=2 Hz, 1H), 8.62 (dd, J=8.5, 2.5 Hz, 1H), 7.96 (s, 1H), 7.68 (d, J=9.0 Hz, 1H), 7.04 (dd, J=8.0, 1.5 Hz, 1H), 6.99 (d, J=1.5 Hz, 1H), 6.75 (d, J=8 Hz, 1H), 5.95 (s, 2H, OCH₂O), 4.18 (m, 2H, CH₂N), 1.72 (m, 2H, CH₂), 1.35 (m, 2H, CH₂), 0.93 (t, J=7.2 Hz, 3H, CH₃).

### Example 14

### Preparation of 5-amino-2-(benzo[d][1,3]dioxol-5-ylthio)-1-butyl-1H-imidazole-4-carboxamide

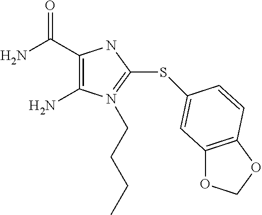

1.8 mL of ethylenediamine were injected over 85 mg (0.17 mmol) of 8-(benzo[d][1,3]dioxol-5-ylthio)-9-butyl-1-(2,4-dinitrophenyl)-1H-purin-6 (9H)-one and the immediate formed dark red solution was stirred at 50° C. for 6 h. Solvent was removed under reduced pressure and coevaporated with toluene. The desired product was isolated by flash column chromatography CH₂Cl₂/MeOH 98:2), yielding 33 mg (0.10 mmol, 60%) of 5-amino-2-(benzo[d][1,3]dioxol-5-ylthio)-1-butyl-1H-imidazole-4-carboxamide as a yellow solid. ¹H-NMR [CD₃OD, δ, ppm]: 6.83 (dd, J=8.0, 1.5 Hz, 1H), 6.80 (d, J=8.0 Hz, 1H), 6.77 (d, J=1.5 Hz, 1H), 5.96 (s, 2H, OCH₂O), 3.89 (t, J=8.0 Hz, 2H, CH₂N), 1.44 (m, 2H, CH₂), 1.29 (m, 2H, CH₂), 0.87 (t, J=7.2 Hz, 3H, CH₃). MS (EI, m/z) 335 (M⁺).

### Example 15

### Preparation of 2-(5-(6-amino-8-(benzo[d][1,3]dioxol-5-ylthio)-9H-purin-9-yl)pentyl)isoindoline-1,3-dione

455 mg (1.58 mmol) of 8-(benzo[d][1,3]dioxol-5-ylthio)-9H-purin-6-amine and 515 mg (1.58 mmol) of Cs₂CO₃ were suspended in distilled DMF (6 mL). Over this suspension, 918 mg (2.37 mmol) of Toluene-4-sulfonic acid 6-(1,3-dioxo-1,3-dihydro-isoindol-2-yl)-pentyl ester (see example 1) were also added and the reaction mixture was stirred at 80° C. for 20 h. Solvent was removed under reduced pressure and the crude was purified by flash chromatography (CH₂Cl₂/EtOAc/MeOH, 100:100:5) to give 414 mg (0.82 mmol, 52%) of 2-(5-(6-amino-8-(benzo[d][1,3]dioxol-5-ylthio)-9H-purin-9-yl)pentyl)isoindoline-1,3-dione as a pale yellow solid. ¹H-NMR [CDCl₃, δ, ppm]: 8.24 (s, 1H), 7.82 (dd, J=5.5, 3.0 Hz, 2H), 7.69 (dd, J=5.5, 3.0 Hz, 2H), 6.99 (dd, J=8.0, 1.5 Hz, 1H), 6.94 (d, J=1.5 Hz, 1H), 6.73 (d, J=8.0 Hz, 1H), 6.02 (br s, 2H), 5.94 (s, 2H, OCH₂O), 4.15 (t, J=7.5 Hz, 2H, CH₂N), 3.64 (t, J=7.5 Hz, 2H, CH₂NCO), 1.77 (m, 2H, CH₂), 1.69 (m, 2H, CH₂), 1.37 (m, 2H, CH₂).

### Example 16

### Preparation of 2-(5-(8-(benzo[d][1,3]dioxol-5-ylthio)-1,6-dihydro-6-oxopurin-9-yl)pentyl)isoindoline-1,3-dione

414 mg (0.82 mmol) of 2-(5-(6-amino-8-(benzo[d][1,3]dioxol-5-ylthio)-9H-purin-9-yl)pentyl)isoindoline-1,3-dione were dissolved in 12 mL of acetic acid. After 10 min stirring, a solution of 569 mg (8.2 mmol) of NaNO₂ in 4 mL of H₂O was injected over the yellow solution. The brown mixture was stirred for 20 h at 25° C. The solvent was removed under reduced pressure and the crude was taken up in a mixture of EtOAc/H₂O. The aqueous phase was extracted twice and the combined organic phases were washed (NaHCO₃, brine) and dried (MgSO₄). Removal of the solvent under reduced pressure and a further purification by Flash chromatography (CH₂Cl₂/EtOAc/MeOH, 100:100:3) yielded 160 mg (0.32 mmol, 39%) of 2-(5-(8-(benzo[d][1,3]dioxol-5-ylthio)-1,6-dihydro-6-oxopurin-9-yl)pentyl)isoindoline-1,3-dione as a white-yellow solid. ¹H-NMR [CDCl₃, δ, ppm]: 7.98 (s, 1H), 7.84 (dd, J=5.5, 3.0 Hz, 2H), 7.71 (dd, J=5.5, 3.0 Hz, 2H), 7.07 (dd, J=8.0, 2.0 Hz, 1H), 7.04 (d, J=2.0 Hz, 1H), 6.76 (d, J=8.0 Hz, 1H), 5.98 (s, 2H, OCH₂O), 4.19 (t, J=7.5 Hz, 2H, CH₂N), 3.66 (t, J=7.0 Hz, 2H, CH₂NCO), 1.80 (m, 2H, CH₂), 1.74 (m, 2H, CH₂), 1.37 (m, 3H, CH₂).

### Example 17

### Preparation of 2-(5-(8-(benzo[d][1,3]dioxol-5-ylthio)-1,6-dihydro-1-(2,4-dinitrophenyl)-6-oxopurin-9-yl)pentyl)isoindoline-1,3-dione

160 mg (0.32 mmol) of 2-(5-(8-(benzo[d][1,3]dioxol-5-ylthio)-1,6-dihydro-6-oxopurin-9-yl)pentyl)isoindoline-1,3-dione, 110 mg (0.80 mmol) of K₂CO₃ and 161 mg (0.80 mmol) of 1-chloro-2,4-dinitrobenzene were dissolved in 5 mL of distilled DMF. The resulting suspension was stirred at 85° C. for 18 h. After removing the solvent under reduced pressure, the crude was purified by Flash chromatography (CH₂Cl₂/EtOAc 1:1 with increasing amounts of methanol until 1%). 180 mg (0.27 mmol, 84%) of 2-(5-(8-(benzo[d][1,3]dioxol-5-ylthio)-1,6-dihydro-1-(2,4-dinitrophenyl)-6-oxopurin-9-yl)pentyl)isoindoline-1,3-dione as a yellow solid were obtained. ¹H-NMR [CDCl₃, δ, ppm]: 9.02 (d, J=2.5 Hz, 1H), 8.65 (dd, J=8.5, 2.5 Hz, 1H), 7.98 (s, 1H), 7.82 (dd, J=5.5, 3.5 Hz, 2H), 7.77 (d, J=9.0 Hz, 1H), 7.71 (dd, J=5.5, 3 Hz, 2H), 7.07 (dd, J=8.0, 2.0 Hz, 1H), 7.02 (d, J=1.0 Hz, 1H), 6.76 (d, J=8.0 Hz, 1H), 5.98 (s, 2H, OCH₂O), 4.25 (m, 2H, CH₂N), 3.66 (t, J=7.2 Hz, 2H, CH₂NCO), 1.85 (m, 2H, CH₂), 1.72 (m, 2H, CH₂), 1.37 (m, 2H, CH₂).

### Example 18

### Preparation of 5-amino-1-(5-aminopentyl)-2-(benzo[d][1,3]dioxol-5-ylthio)-1H-imidazole-4-carboxamide

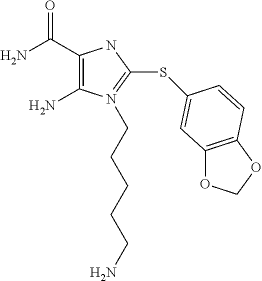

2.8 mL of ethylenediamine were injected over 170 mg (0.25 mmol) of 2-(5-(8-(benzo[d][1,3]dioxol-5-ylthio)-1,6-dihydro-1-(2,4-dinitrophenyl)-6-oxopurin-9-yl)pentyl)isoindoline-1,3-dione and the immediate formed dark red solution was stirred at 50° C. for 6 h. Solvent was removed under reduced pressure and coevaporated with toluene. The desired product was isolated by flash column chromatography (CH₂Cl₂/MeOH/NH₄OH 8:2:0.1), yielding 43 mg (0.12 mmol, 47%) of a brown oil as the desired product 5-amino-1-(5-aminopentyl)-2-(benzo[d][1,3]dioxol-5-ylthio)-1H-imidazole-4-carboxamide. ¹H-NMR [CD₃OD, δ, ppm]: 6.83 (dd, J=8.5, 1.5 Hz, 1H), 6.79 (d, J=8.5 Hz, 1H), 6.77 (d, J=1.5 Hz, 1H), 5.96 (s, 2H, OCH₂O), 3.90 (t, J=7.7 Hz, 2H, CH₂N), 2.57 (t, J=7.2 Hz, 2H, CH₂N), 1.49 (m, 2H, CH₂), 1.40 (m, 2H, CH₂), 1.26 (m, 2H, CH₂). MS (EI, m/z) 364 (M⁺).

### Example 19

### Preparation of 5-acetamido-2-(benzo[d][1,3]dioxol-5-ylthio)-1-butyl-1H-imidazole-4-carboxamide

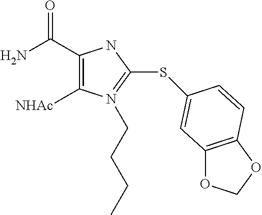

Acetic anhydride (4.2 μL, 0.045 mmol) was added to a solution of 5-amino-2-(benzo[d][1,3]dioxol-5-ylthio)-1-butyl-1H-imidazole-4-carboxamide (7.5 mg, 0.022 mmol) in acetic acid (1.2 mL). The reaction mixture was heated at 120° C. for 2 h. Solvent was removed and purification of the crude by flash column chromatography (CH₂Cl₂/MeOH 98:2) afforded the desired product 5-acetamido-2-(benzo[d][1,3]dioxol-5-ylthio)-1-butyl-1H-imidazole-4-carboxamide (2.7 mg, 32%) as colourless oil. ¹H-NMR [CD₃OD, δ, ppm]: 6.92 (d, J=8.0, 1H), 6.88 (s, 1H), 6.82 (d, J=8.0 Hz, 1H), 5.97 (s, 2H, OCH₂O), 3.94 (t, J=7.7 Hz, 2H, CH₂N), 2.17 (s, 3H, CH₃CO), 1.51 (m, 2H, CH₂), 1.27 (m, 2H, CH₂), 0.88 (t, J=7.2 Hz, 3H, CH₃). MS (EI, m/z) 377 (M⁺).

### Example 20

### Preparation of 8-(4-(trifluoromethyl)phenylthio)-9H-purin-6-amine

A mixture of 400 mg (2.4 mmol) of 6-amino-9H-purine-8-thiol (cf. Llauger et al., “Evaluation of 8-Arylsulfanyl, 8-Arylsulfoxyl, and 8-Arylsulfonyl Adenine Derivatives as Inhibitors of the Heat Shock Protein 90”, J. Med. Chem., 2005, vol. 48, p. 2892), 50 mg (0.24 mmol) of neocuproine, 46 mg (0.24 mmol) of Cul and 584 mg (4.8 mmol) of potassium tert-butoxide was suspended in 10 mL of anhydrous DMF. Over the white suspension, 1.95 g (7.2 mmol) of 4-iodobenzotrifluoride, dissolved in 7 mL anhydrous DMF, were also added. The crude was stirred for 20 h at 120° C. Removal of the solvent under high vacuum afforded a dark brown residue that was purified by flash chromatography (CH₂Cl₂/EtOAc/MeOH, 2:2:0.5) to give 620 mg, (1.99 mmol, 83%) of 8-(4-(trifluoromethyl)phenylthio)-9H-purin-6-amine as a pale brown solid. ¹H-NMR [CDCl₃/CD₃OD, δ, ppm]: 8.12 (s, 1H), 7.54 (d, J=8.5 Hz, 2H), 7.54 (d, J=8.5 Hz, 2H).

### Example 21

### Preparation of 8-(4-(trifluoromethyl)phenylthio)-9-butyl-9H-purin-6-amine

150 mg (0.48 mmol) of 8-(4-(trifluoromethyl)phenylthio)-9H-purin-6-amine and 156 mg (0.48 mmol) of Cs₂CO₃ were suspended in distilled DMF (1.5 mL). Over this suspension, 165 mg (0.72 mmol) of butyl-4-methylbenzenesulfonate (cf. Sekera, V. et al., “Higher alkyl sulfonates”, J. Am. Chem. Soc., 1993, vol. 55, p. 345) were also added and the reaction mixture was stirred at 80° C. for 20 h. Solvent was removed under reduced pressure and the black residue was purified by flash chromatography (CH₂Cl₂/MeOH, 100:3) to give 90 mg (0.24 mmol, 51%) of 8-(4-(trifluoromethyl)phenylthio)-9-butyl-9H-purin-6-amine as a yellowish solid. ¹H-NMR [CDCl₃, δ, ppm]: 8.35 (s, 1H), 7.55 (d, J=8.0 Hz, 2H), 7.42 (d, J=8.0 Hz, 2H), 6.00 (br s, 2H), 4.20 (t, J=7.5 Hz, 2H, CH₂N), 1.71 (m, 2H, CH₂), 1.33 (m, 2H, CH₂), 0.89 (t, J=7.2 Hz, 3H, CH₃).

### Example 22

### Preparation of 8-(4-(trifluoromethyl)phenylthio)-9-butyl-1H-purin-6 (9H)-one

8-(4-(Trifluoromethyl)phenylthio)-9-butyl-9H-purin-6-amine (150 mg, 0.41 mmol) was dissolved in 6 mL of acetic acid. After 10 min stirring, a solution of 282 mg (4.1 mmol) of NaNO₂ in 2 mL of H₂O was injected over the yellow solution. The brown mixture was stirred for 20 h at 25° C. The solvent was removed under reduced pressure and the crude was taken up in a mixture of EtOAc/H₂O. The aqueous phase was extracted twice and the combined organic phases were washed (NaHCO₃, brine) and dried (MgSO₄). Removal of the solvent under reduced pressure and a further purification by flash chromatography (CH₂Cl₂/EtOAc/MeOH, 100:100:3) yielded 80 mg (0.22 mmol, 53%) of 8-(4-(trifluoromethyl)phenylthio)-9-butyl-1H-purin-6 (9H)-one as a yellowish solid. ¹H-NMR [CDCl₃, δ, ppm]: 8.10 (s, 1H), 7.57 (s, 4H), 4.22 (t, J=7.5 Hz, 2H, CH₂N), 1.73 (m, 2H, CH₂), 1.32 (m, 2H, CH₂), 0.91 (t, J=7.2 Hz, 3H, CH₃).

### Example 23

### Preparation of 1-[(2-methoxyethoxy)methyl]-8-(4-(trifluoromethyl)phenylthio)-9-butyl-1H-purin-6 (9H)-one

8-[4-(Trifluoromethyl)phenylthio]-9-butyl-1H-purin-6(9H)-one (108 mg, 0.29 mmol) were dissolved in 2 mL of anhydrous CH₂Cl₂. Over this solution, 76 μL (0.44 mmol) of diisopropylethylamine were injected and the mixture was stirred for 5 min. Then, 2-methoxyethoxymethyl chloride (40 μL, 0.35 mmol) was also injected dropwise. The reaction mixture was stirred for 20 h kept from light. H₂O was added to quench the reaction and the aqueous phase was extracted with CH₂Cl₂. The combined organic phases were washed with HCl 0.05 N and NaHCO₃. Finally, the organic phase was dried (MgSO₄), filtered and evaporated under vacuum, giving a white paste that was purified by flash chromatography (CH₂Cl₂/EtOAc/MeOH, 100:100:3) yielding 102 mg (0.22 mmol, 76%) of 1-[(2-methoxyethoxy)methyl]-8-(4-(trifluoromethyl)phenylthio)-9-butyl-1H-purin-6(9H)-one as a yellow oil. ¹H-NMR [CDCl₃, δ, ppm]: 8.12 (s, 1H), 7.55 (d, J=8.5 Hz, 2H), 7.50 (d, J=8.0 Hz, 2H), 5.56 (s, 2H, OCH₂N), 4.18 (t, J=7.5 Hz, 2H, CH₂N), 3.83 (m, 2H, CH₂O), 3.53 (m, 2H, CH₂O), 3.36 (s, 3H, CH₃O), 1.69 (m, 2H, CH₂), 1.30 (m, 2H, CH₂), 0.90 (t, J=7.2 Hz, 3H, CH₃).

### Example 24

### Preparation of 2-(4-(trifluoromethyl)phenylthio)-5-amino-1-butyl-1H-imidazole-4-carboxamide

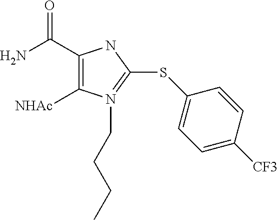

68 mg (0.15 mmol) of 1-[(2-methoxyethoxy)methyl]-8-(4-(trifluoromethyl)phenylthio)-9-butyl-1H-purin-6 (9H)-one and 2 mL of NaOH 0.2N were stirred at reflux temperature for 20 h. The solution was cooled and neutralized with HCl 3-4N until pH 6. Solvent was removed under reduced pressure and the crude was purified by flash column chromatography (CHCl₃/MeOH, 98:2 to 96:4). 11 mg (0.03 mmol, 21%) of 2-(4-(trifluoromethyl)phenylthio)-5-amino-1-butyl-1H-imidazole-4-carboxamide were obtained as a colourless oil. ¹H-NMR [CD₃OD, δ, ppm]: 7.61 (d, J=8.5 Hz, 2H), 7.29 (d, J=8.0 Hz, 1H), 3.89 (t, J=7.5 Hz, 2H, CH₂N), 1.50 (m, 2H, CH₂), 1.25 (m, 2H, CH₂), 0.83 (t, J=7.5 Hz, 3H, CH₃). MS (EI, m/z) 359 (M⁺).

### Example 25

### Preparation of 8-(2,5-dimethoxyphenylthio)-9-butyl-1H-purin-6 (9H)-one

This compound was obtained according to Biamonte et al., “Orally Active Purine-Based Inhibitors of the Heat Shock Protein 90”, J. Med. Chem. 2006, vol. 49, p. 817.

230 mg (0.64 mmol) of 9-Butyl-8-(2,5-dimethoxy-phenylsulfanyl)-9H-purin-6-ylamine were dissolved in 9 mL of acetic acid. After 10 min stirring, a solution of 440 mg (6.4 mmol) of NaNO₂ in 3 mL of H₂O was injected over the yellow solution. The brown mixture was stirred for 20 h at 25° C. The solvent was removed under reduced pressure and the crude was taken up in a mixture of EtOAc/H₂O. The aqueous phase was extracted twice and the combined organic phases were washed (NaHCO₃, brine) and dried (MgSO₄). Removal of the solvent under reduced pressure and a further purification by flash chromatography (CH₂Cl₂/EtOAc/MeOH, 100:100:3) yielded 180 mg (0.50 mmol, 79%) of 8-(2,5-dimethoxyphenylthio)-9-butyl-1H-purin-6 (9H)-one as a white solid. ¹H-NMR [CDCl₃, δ, ppm]: 8.07 (s, 1H), 6.85 (d, J=2.5 Hz, 1H), 6.83 (d, J=9.0 Hz, 1H), 6.79 (dd, J=9.0, 2.5 Hz, 1H), 4.24 (t, J=7.5 Hz, 2H, CH₂N), 3.78 (s, 3H, CH₃O), 3.69 (s, 3H, CH₃O), 1.74 (m, 2H, CH₂), 1.33 (m, 2H, CH₂), 0.92 (t, J=7.2 Hz, 3H, CH₃).

### Example 26

### Preparation of 1-[(2-methoxyethoxy)methyl]-8-(2,5-dimethoxyphenylthio)-9-butyl-1H-purin-6 (9H)-one

8-(2,5-Dimethoxyphenylthio)-9-butyl-1H-purin-6 (9H)-one (178 mg, 0.49 mmol) were dissolved in 3.5 mL of anhydrous CH₂Cl₂. Over this solution, 129 μL (0.74 mmol) of diisopropylethylamine were injected and the mixture was stirred for 5 min. Then, 2-methoxyethoxymethyl chloride (68 μL, 0.59 mmol) was also injected dropwise. The reaction mixture was stirred for 20 h kept from light. H₂O was added to quench the reaction and the aqueous phase was extracted with CH₂Cl₂ The combined organic phases were washed with HCl 0.05N and NaHCO₃. Finally, the organic phase was dried (MgSO₄), filtered and evaporated under vacuum, giving a white paste that was purified by flash chromatography (CH₂Cl₂/EtOAc/MeOH, 100:100:3) yielding 165 mg (0.37 mmol, 75%) of 1-[(2-methoxyethoxy)methyl]-8-(2,5-dimethoxyphenylthio)-9-butyl-1H-purin-6 (9H)-one as a yellow oil. ¹H-NMR [CDCl₃, δ, ppm]: 8.09 (s, 1H), 6.81 (dd, J=5.5, 4.5 Hz, 1H), 6.76 (m, 2H), 5.54 (s, 2H, OCH₂N), 4.19 (t, J=7.5 Hz, 2H, CH₂N), 3.81 (m, 2H, CH₂O), 3.79 (s, 3H, CH₃O), 3.69 (s, 3H, CH₃O), 3.52 (m, 2H, CH₂O), 3.34 (s, 3H, CH₃O), 1.69 (m, 2H, CH₂), 1.30 (m, 2H, CH₂), 0.89 (t, J=7.2 Hz, 3H, CH₃).

### Example 27

### Preparation of 5-amino-1-butyl-2-(2,5-dimethoxy-phenylsulfanyl)-1H-imidazole-4-carboxamide

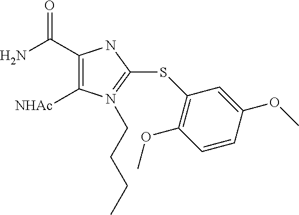

150 mg (0.33 mmol) of 1-[(2-methoxyethoxy)methyl]-8-(2,5-dimethoxyphenylthio)-9-butyl-1H-purin-6 (9H)-one and 5 mL of NaOH 0.2N were stirred at reflux temperature for 20 h. The solution was cooled and neutralized with HCl 3-4N until pH 6. Solvent was removed under reduced pressure and the crude was purified by flash column chromatography (CHCl₃/MeOH, 98:2 to 96:4). 32 mg (0.03 mmol, 21%) of 5-Amino-1-butyl-2-(2,5-dimethoxy-phenylsulfanyl)-1H-imidazole-4-carboxamide were obtained as a yellowish solid. ¹H-NMR [CD₃OD, δ, ppm]: 6.92 (d, J=9.0 Hz, 1H), 6.76 (dd, J=9.0, 3.0 Hz, 1H), 6.28 (d, J=3.0 Hz, 1H), 3.88 (t, J=7.5 Hz, 2H, CH₂N), 3.83 (s. 3H, CH₃O), 3.64 (s, 3H, CH₃O), 1.51 (m, 2H, CH₂), 1.26 (m, 2H, CH₂), 0.86 (t, J=7.5 Hz, 3H, CH₃). MS (EI, m/z) 351 (M⁺).

### Example 28

### Preparation of 5-acetylamino-1-butyl-2-(6-iodo-benzo[1,3]dioxol-5-ylsulfanyl)-1H-imidazole-4-carboxamide

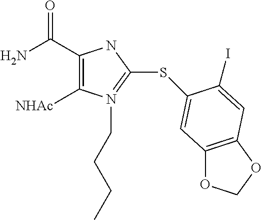

Acetic anhydride (10 μL, 0.1 mmol) was added to a solution of 5-Amino-1-butyl-2-(6-iodo-benzo[1,3]dioxol-5-ylsulfanyl)-1H-imidazole-4-carboxylamide (43 mg, 0.09 mmol) in acetic acid (12 mL). The reaction mixture was heated at 120° C. for 2 h. Solvent was removed and purification of the crude by flash column chromatography (CH₂Cl₂/MeOH 98:2) afforded the desired product 5-Acetylamino-1-butyl-2-(6-iodo-benzo[1,3]dioxol-5-ylsulfanyl)-1H-imidazole-4-carboxamide (30 mg, 65%) as colourless oil. ¹H-NMR [CDCl₃, δ, ppm]: 7.20 (s, 1H), 6.53 (s, 1H), 6.82 (d, J=8.0 Hz, 1H), 5.92 (s, 2H, OCH₂O), 4.08 (t, J=7.5 Hz, 2H, CH₂N), 2.21 (s, 3H, CH₃CO), 1.55 (m, 2H, CH₂), 1.21 (m, 2H, CH₂), 0.84 (t, J=7.2 Hz, 3H, CH₃). MS (EI, m/z) 503 (M⁺+1).

### Example 29

### Preparation of {3-[6-amino-8-(6-iodo-benzo[1,3]dioxol-5-ylsulfanyl)-purin-9-yl]-propyl}-isopropyl-carbamic acid tert-butyl ester

A solution of 8-(6-iodo-benzo[1,3]dioxol-5-ylsulfanyl)-9H-purin-6-ylamine previously prepared (cf. Chiosis G. et al., “Identification of Potent Water Soluble Purine-Scaffold Inhibitors of the Heat Shock Protein 90”, J. Med. Chem. 2006, vol. 49, no. 1, p. 381) (0.4 g, 0.9 mmol), toluene-4-sulfonic acid 1-(tert-butoxycarbonyl-isopropyl-amino)-propyl ester previously prepared (cf. Llauger et al., “Evaluation of 8-Arylsulfanyl, 8-Arylsulfoxyl, and 8-Arylsulfonyl Adenine Derivatives as Inhibitors of the Heat Shock Protein 90”, J. Med. Chem. 2005, vol. 48, p. 2892) (0.8 g, 2.2 mmol), and Cs₂CO₃ (0.8 g, 2.4 mmol) in 8 mL of DMF was stirred at 80° C. for 16 h. Removal of the solvent and purification by chromatography on silica (CHCl₃/MeOH, 5%) yielded {3-[6-Amino-8-(6-iodo-benzo[1,3]dioxol-5-ylsulfanyl)-purin-9-yl]-propyl}-isopropyl-carbamic acid tert-butyl ester (46 mg, 10%) as a viscous oil. ¹H-NMR [CDCl₃, δ, ppm]: 8.34 (s, 1H), 7.19 (m, 1H), 6.89 (s, 1H), 5.97 (s, 2H, OCH₂O), 4.25 (m, 2H, CH₂N), 2.07 (m, 1H, CH), 2.04 (m, 4H, 2CH₂), 1.48 (m, 9H, 3CH₃), 1.12 (d, J=6.9 Hz, 6H).

### Example 30

### Preparation of {3-[8-(6-iodo-benzo[1,3]dioxol-5-ylsulfanyl)-6-oxo-1,6-dihydro-purin-9-yl]-propyl}-isopropyl-carbamic acid tert-butyl ester

A solution of {3-[6-amino-8-(6-iodo-benzo[1,3]dioxol-5-ylsulfanyl)-purin-9-yl]-propyl}-isopropyl-carbamic acid tert-butyl ester (46 mg, 0.07 mmol) in HOAc (4 mL) was stirred at room temperature, and then NaNO₂ (71 mg, 1 mmol) in H₂O (2 mL) was added. Then the reaction mixture was stirred at room temperature for 16 h. The solvent was removed and water was added to the residue. The aqueous phase was extracted with AcOEt. The organic phase was washed (NaHCO₃, brine) and dried (MgSO₄). Removal of the solvent and purification by chromatography on silica (CHCl₃/AcOEt/MeOH, 5:4:1) yielded {3-[8-(6-Iodo-benzo[1,3]dioxol-5-ylsulfanyl)-6-oxo-1,6-dihydro-purin-9-yl]-propyl}-isopropyl-carbamic acid tert-butyl ester (31 mg, 67%) as a foam. ¹H-NMR [CDCl₃, δ, ppm]: 8.20 (s, 1H), 7.27 (m, 1H), 6.98 (s, 1H), 5.93 (s, 2H, OCH₂O), 4.25 (m, 2H, CH₂N), 2.07 (m, 1H, CH), 2.04 (m, 4H, 2CH₂), 1.48 (m, 9H, 3CH₃), 1.12 (d, J=6.9 Hz, 6H).

### Example 31

### Preparation of {3-[1-(2,4-dinitro-phenyl)-8-(6-iodo-benzo[1,3]dioxol-5-ylsulfanyl)-6-oxo-1,6-dihydro-purin-9-yl]-propyl}-isopropyl-carbamic acid tert-butyl ester

{3-[8-(6-iodo-benzo[1,3]dioxol-5-ylsulfanyl)-6-oxo-1,6-dihydro-purin-9-yl]-propyl}-isopropyl-carbamic acid tert-butyl ester (31 mg, 0.05 mmol), K₂CO₃ (17 mg, 0.12 mmol) and 1-chloro-2,4-dinitrobenzene (26 mg, 0.12 mmol) were dissolved in 4 mL of distilled DMF. The resulting suspension was stirred at 85° C. for 18 h. After removing the solvent under reduced pressure, the crude was purified by flash chromatography (CHCl₃ 100%) {3-[1-(2,4-dinitro-phenyl)-8-(6-iodo-benzo[1,3]dioxol-5-ylsulfanyl)-6-oxo-1,6-dihydro-purin-9-yl]-propyl}-isopropyl-carbamic acid tert-butyl ester (30 mg, 77%) of as a foam were obtained. ¹H-NMR [CDCl₃, δ, ppm]: 9.10 (s, 1H), 8.56 (s, 1H), 8.00 (s, 1H), 8.16 (s, 1H), 7.27 (m, 1H), 6.98 (s, 1H), 5.93 (s, 2H, OCH₂O), 4.25 (m, 2H, CH₂N), 2.07 (m, 1H, CH), 2.04 (m, 4H, 2CH₂), 1.48 (m, 9H, 3CH₃), 1.12 (d, J=6.9 Hz, 6H).

### Example 32

### Preparation of {3-[5-amino-4-carbamoyl-2-(6-iodo-benzo[1,3]dioxol-5-ylsulfanyl)-imidazol-1-yl]-propyl}-isopropyl-carbamic acid tert-butyl ester

3 mL of ethylenediamine were injected over 3-[1-(2,4-dinitro-phenyl)-8-(6-iodo-benzo[1,3]dioxol-5-ylsulfanyl)-6-oxo-1,6-dihydro-purin-9-yl]-propyl}-isopropyl carbamic acid tert-butyl ester 30 mg (0.04 mmol) of and the immediate formed dark red solution was stirred at 50° C. for 6 h. Solvent was removed under reduced pressure and co evaporated with toluene. The desired product was isolated by flash column chromatography (CHCl₃:MeOH 3%), yielding (20 mg, 87%) of a brown oil as the desired product {3-[5-Amino-4-carbamoyl-2-(6-iodo-benzo[1,3]dioxol-5-ylsulfanyl)-imidazol-1-yl]-propyl}-isopropyl-carbamic acid tert-butyl ester. ¹H-NMR [CDCl₃, δ, ppm]: 7.15 (m, 1H), 6.47 (s, 1H), 5.90 (s, 2H, OCH₂O), 3.93 (m, 2H, CH₂N), 2.86 (m, 1H, CH), 2.08 (m, 4H, 2CH₂), 1.48 (m, 9H, 3CH₃), 1.27 (d, J=6.9 Hz, 6H).

### Example 33

### Preparation of 5-amino-2-(6-iodo-benzo[1,3]dioxol-5-ylsulfanyl)-1-(3-isopropylamino-propyl)-1H-imidazole-4-carboxamide

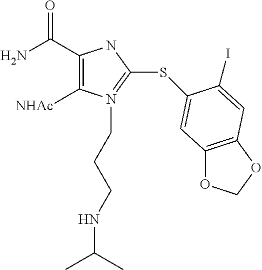

To a solution of {3-[5-amino-4-carbamoyl-2-(6-iodo-benzo[1,3]dioxol-5-ylsulfanyl)-imidazol-1-yl]-propyl}-isopropyl-carbamic acid tert-butyl ester (20 mg, 0.03 mmol) in 1 mL of CH₂Cl₂ was added 0.2 mL of trifluoroacetic acid (hereinafter abbreviated as “TFA”). This reaction mixture was stirred at room temperature for 16 h. Removal of the solvent and purification by chromatography on silica (CHCl₃/MeOH/NH₄OH, 90:10:2) yielded 5-amino-2-(6-iodo-benzo[1,3]dioxol-5-ylsulfanyl)-1-(3-isopropylamino-propyl)-1H-imidazole-4-carboxamide (5 mg, 31%) as a viscous oil. ¹H-NMR [CDCl₃, 6, ppm]: 7.15 (m, 1H), 6.47 (s, 1H), 5.90 (s, 2H, OCH₂O), 3.93 (m, 2H, CH₂N), 2.86 (m, 1H, CH), 2.08 (m, 4H, 2CH₂), 1.27 (d, J=6.9 Hz, 6H). MS (EI, m/z) 504 (M⁺+1).

### Example 34

### Preparation of 6-(toluene-4-sulfonyloxy)-hexanoic acid methyl ester

A solution of 6-hydroxy-hexanoic acid methyl ester previously prepared (cf. EI Fangour S. et al., “Total synthesis of phytoprostane F₁ and its 16 epimer”, Tetrahedron Letters, 2003, vol. 44, p. 2105) (4.6 g, 31 mmol), p-toluensulfonyl chloride (7 g, 36.7 mmol), and pyridine (3 mL, 37.1 mmol) in 70 mL of CH₂Cl₂ was stirred at room temperature for 16 h. Removal of the solvent and purification by chromatography on silica (hexane/AcOEt, 4:1) yielded 6-(toluene-4-sulfonyloxy)-hexanoic acid methyl ester (1.24 g, 12%) as a viscous oil. ¹H-NMR [CDCl₃, δ, ppm]: 7.79 (d, J=5 Hz, 2H), 7.36 (d, J=5 Hz, 2H), 4.03 (t, J=5 Hz, 2H, CH₂O), 3.67 (s, 3H, COOCH₃), 2.46 (s, 3H, CH₃), 2.26 (t, J=5 Hz, 2H, CH₂COOO), 1.67 (m, 2H, CH₂), 1.58 (m, 2H, CH₂), 1.37 (m, 2H, CH₂).

### Example 35

### Preparation of 6-[6-amino-8-(6-iodo-benzol[1,3]dioxol-5-ylsulfanyl)-purin-9-yl]-hexanoic acid methyl ester

A solution of 8-(6-iodo-benzo[1,3]dioxol-5-ylsulfanyl)-9H-purin-6-ylamine previously prepared (cf. Chiosis G. et al., “Identification of Potent Water Soluble Purine-Scaffold Inhibitors of the Heat Shock Protein 90”, J. Med. Chem. 2006, vol. 49, no. 1, p. 381) (0.3 g, 0.7 mmol), 6-(Toluene-4-sulfonyloxy)-hexanoic acid methyl ester (0.5 g, 1.7 mmol), and Cs₂CO₃ (0.24 g, 0.7 mmol) in 6 mL of DMF was stirred at 80° C. for 16 h. Removal of the solvent and purification by chromatography on silica (CHCl₃/MeOH, 5%) yielded 6-[6-Amino-8-(6-iodo-benzo[1,3]dioxol-5-ylsulfanyl)-purin-9-yl]-hexanoic acid methyl ester (85 mg, 21%) as a viscous oil. ¹H-NMR [CDCl₃, δ, ppm]: 8.32 (s, 1H), 7.18 (s, 1H), 6.88 (s, 1H), 5.99 (s, 2H, OCH₂O), 4.20 (m, 2H, CH₂N), 3.62 (s, 3H, COOCH₃), 2.26 (t, J=5 Hz, 2H, CH₂COO), 1.67 (m, 2H, CH₂), 1.58 (m, 2H, CH₂), 1.37 (m, 2H, CH₂).

### Example 36

### Preparation of 6-[6-amino-8-(6-iodo-benzol[1,3]dioxol-5-ylsulfanyl)-purin-9-yl]-hexanoic acid

6-[6-amino-8-(6-iodo-benzo[1,3]dioxol-5-ylsulfanyl)-purin-9-yl]-hexanoic acid methyl ester (85 mg, 0.16 mmol) was treated with 5 mL of 1N LiOH (MeOH:H₂O (1:1)) in 2 mL dioxane at room temperature for 16 h. The reaction mixture was concentrated to dryness. The residue was dissolved in the minimum amount of water and acidified to pH=2. The resulting precipitate was collected, washed with water and dried yielded 6-[6-amino-8-(6-iodo-benzo[1,3]dioxol-5-ylsulfanyl)-purin-9-yl]-hexanoic acid (73 mg, 89%) as a foam. ¹H-NMR [CDCl₃, δ, ppm]: 8.32 (s, 1H), 7.49 (s, 1H), 7.30 (s, 1H), 6.10 (s, 2H, OCH₂O), 4.35 (m, 2H, CH₂N), 2.32 (t, J=5 Hz, 2H, CH₂COO), 1.67 (m, 2H, CH₂), 1.58 (m, 2H, CH₂), 1.37 (m, 2H, CH₂).

### Example 37

### Preparation of 6-[6-amino-8-(6-iodo-benzol[1,3]dioxol-5-ylsulfanyl)-purin-9-yl]-hexanoic acid 4,4-difluoro-5,7-dimethyl-4-bora-3a,4a-diaza-s-indacene-3-propionylethylenediamide (used as a tracer in example 42)

6-[6-amino-8-(6-iodo-benzo[1,3]dioxol-5-ylsulfanyl)-purin-9-yl]-hexanoic acid (3 mg, 5.6 μmol), 3,4-dihydro-3-hydroxy-4-oxo-1,2,3-benzotriazine (1 mg, 6 μmol), and N,N′-diisopropylcarbodiimide (0.1 mL, 7 mmol) were combined in a flask and dissolved in 1 mL DMF. The mixture was stirred under argon for 1 h at room temperature, and, then, N-ethylmorpholine (0.1 mL, 12 mmol) and 4,4-difluoro-5,7-dimethyl-4-bora-3a,4a-diaza-s-indacene-3-propionyl ethylenediamine hydrochloride (2 mg, 5 μmol) in 1 mL DMF were added to the mixture. After stirring for 16 h at room temperature, the solvents were removed under vacuum. The residue was purified by chromatography on alumina (CH₂Cl₂/MeOH, 2%) yielded 6-[6-amino-8-(6-iodo-benzo[1,3]dioxol-5-ylsulfanyl)-purin-9-yl]-hexanoic acid 4,4-difluoro-5,7-dimethyl-4-bora-3a,4a-diaza-s-indacene-3-propionylethylenediamide (2.8 mg, 58%) as a viscous oil. ¹H-NMR [CDCl₃, δ, ppm]: 8.32 (s, 1H), 7.49 (s, 1H), 7.30 (s, 1H), 6.28 (m, 2H), 6.14 (m, 2H), 6.10 s, 2H, OCH₂O), 4.35 (m, 2H, CH₂N), 3.50 (m, 4H, NCH₂CH₂N), 2.68 (m, 2H, COCH₂), 2.32 (t, J=5 Hz, 2H, CH₂COO), 1.78 (m, 2H, CH₂), 1.67 (m, 2H, CH₂), 1.58 (m, 2H, CH₂), 1.37 (m, 2H, CH₂). MS (EI, m/z) 844 (M⁺+1).

### Example 38

### Preparation of toluene-4-sulfonic acid 6-(1,3-dioxo-1,3-dihydro-isoindol-2-yl)-butyl ester

1,8-naphthalic anhydride (3.3 g, 22 mmol) was added to a solution 4-amino-butan-1-ol (2 g, 22 mmol) in absolute ethanol (100 mL). The reaction mixture was heated at reflux overnight. Removal of the solvent and purification by chromatography on alumina (CH₂Cl₂/MeOH, 50:1) yielded 2-(6-hydroxy-butyl)-isoindole-1,3-dione (4.7 g, 90%) as a foam. ¹H-NMR [CDCl₃, δ, ppm]: 8.62 (m, 2H), 8.22 (m, 2H), 3.69 (m, 2H, CH₂O), 3.58 (m, 2H, CH₂NO), 1.72 (m, 2H, CH₂), 1.41 (m, 2H, CH₂).

A solution of 2-(6-hydroxy-butyl)isoindole-1,3-dione (4.7 g, 22 mmol), p-toluensulfonyl chloride (5 g, 26 mmol), and pyridine (2 mL, 25 mmol) in 70 mL of CH₂Cl₂ was stirred at room temperature for 16 h. Removal of the solvent and purification by chromatography on silica (hexane/CH₂Cl₂/AcOEt, 5:4:1) yielded toluene-4-sulfonic acid 6-(1,3-dioxo-1,3-dihydro-isoindol-2-yl)-butyl ester (2.4 g, 29%) as a viscous oil. ¹H-NMR [CDCl₃, δ, ppm]:, 8.62 (m, 2H), 8.22 (m, 2H), 7.79 (d, J=5 Hz, 2H), 7.36 (d, J=5 Hz, 2H, 4.02 (m, 2H, CH₂O), 3.58 (m, 2H, CH₂NO), 2.46 (s, 3H, CH₃), 1.72 (m, 2H, CH₂), 1.41 (m, 2H, CH₂).

### Example 39

### Preparation of 2-{-4-[6-amino-8-(6-iodo-benzol[1,3]dioxol-5-ylsulfanyl)-purin-9-yl]-butyl}-isoindole-1,3-dione

A solution of 8-(6-iodo-benzo[1,3]dioxol-5-ylsulfanyl)-9H-purin-6-ylamine previously prepared (cf. Chiosis G. et al., “Identification of Potent Water Soluble Purine-Scaffold Inhibitors of the Heat Shock Protein 90”, J. Med. Chem., 2006, vol. 49, no. 1, p. 381 (0.3 g, 0.7 mmol), toluene-4-sulfonic acid 6-(1,3-dioxo-1,3-dihydro-isoindol-2-yl)-butyl ester (0.4 g, 1 mmol), and Cs₂CO₃ (0.2 g, 0.7 mmol) in 5 mL of DMF was stirred at 80° C. for 16 h. Removal of the solvent and purification by chromatography on silica (CHCl₃/AcOEt/MeOH, 5:4:1) yielded 2-{4-[6-amino-8-(6-iodo-benzo[1,3]dioxol-5-ylsulfanyl)-purin-9-yl]-butyl}-isoindole-1,3-dione (0.4 g, 98%) as a viscous oil. ¹H-NMR [CDCl₃, δ, ppm]: 8.32 (s, 1H), 7.81 (m, 2H), 7.68 (m, 2H), 7.49 (s, 1H), 7.30 (s, 1H), 5.99 (s, 2H, OCH₂O), 4.16 (m, 2H, CH₂N), 3.64 (m, 2H, CH₂NO), 1.72 (m, 2H, CH₂), 1.41 (m, 2H, CH₂).

### Example 40

### Preparation of 9-(4-amino-butyl)-8-(6-iodo-benzol[1,3]dioxol-5-ylsulfanyl)-9H-purin-6-ylamine

A 33% solution of methylamine in absolute ethanol (13 mL) is added to a stirred suspension of 2-{4-[6-amino-8-(6-iodo-benzo[1,3]dioxol-5-ylsulfanyl)-purin-9-yl]-butyl}-isoindole-1,3-dione (45 mg, 0.72 mmol) in EtOH (17 mL) at room temperature. After 5 min, the clear solution obtained was refluxed for 16 h. and the mixture was cooled at room temperature. Removal of the solvent and purification by chromatography on silica (CHCl₃/MeOH/NH₄OH, 5:1:0.5) yielded 9-(4-amino-butyl)-8-(6-iodo-benzo[1,3]dioxol-5-ylsulfanyl)-9H-purin-6-ylamine (0.17 g, 49%) as a foam. ¹H-NMR [CDCl₃, δ, ppm]: 8.32 (s, 1H), 7.49 (s, 1H), 7.30 (s, 1H), 5.99 (s, 2H, OCH₂O), 4.18 (m, 2H, CH₂N), 2.70 (m, 2H, CH₂NH₂), 1.72 (m, 2H, CH₂), 1.41 (m, 2H, CH₂).

### Example 41

### Preparation of 6-(7-nitro-benzo[1,2,5]oxadiazol-4-ylamino)-hexanoic acid {-4-[6-amino-8-(6-iodo-benzol[1,3]dioxol-5-ylsulfanyl)-purin-9-yl]-butyl}-amide (used as a tracer in example 42)

9-(4-amino-butyl)-8-(6-iodo-benzo[1,3]dioxol-5-ylsulfanyl)-9H-purin-6-ylamine (0.1 g, 0.2 mmol), 3,4-dihydro-3-hydroxy-4-oxo-1,2,3-benzotriazine (37 mg, 0.2 mmol), and N,N′-diisopropylcarbodiimide (0.1 mL, 0.7 mmol) were combined in a flask and dissolved in 10 mL DMF. The mixture was stirred under argon for 1 h at room temperature, and, then, N-ethylmorpholine (0.1 mL, 0.8 mmol) and 6-(7-nitrobenzofurazan-4-ylamino)hexanoic acid (56 mg, 0.2 mmol) in 10 mL of DMF were added to the mixture. After stirring for 16 h at room temperature, the solvents were removed under vacuum. The residue was purified by chromatography on alumina (CH₂Cl₂/MeOH, 2%) yielded 6-(7-nitro-benzo[1,2,5]oxadiazol-4-ylamino)-hexanoic acid {-4-[6-amino-8-(6-iodo-benzo[1,3]dioxol-5-ylsulfanyl)-purin-9-yl]-butyl}-amide (45 mg, 30%) as a viscous oil. ¹H-NMR [CDCl₃, δ, ppm]: 8.48 (m, 1H), 7.95 (s, 1H), 6.88 (s, 1H), 6.67 (m, 1H), 6.35 (m, 1H), 6.10 (s, 2H, OCH₂O), 4.24 (m, 2H, CH₂N), 3.35 (m, 2H, CH₂NHCO), 2.24 (m, 2H, CH₂N), 1.77 (m, 2H, CH₂CO), 1.82 (m, 4H, CH₂), 1.67 (m, 2H, CH₂), 1.58 (m, 2H, CH₂), 1.37 (m, 2H, CH₂). MS (EI, m/z) 783 (M⁺+23).

### Example 42

### Preparation of {3-[8-(6-iodo-benzo[1,3]dioxol-5-ylmethyl)-6-oxo-1,6-dihydro-purin-9-yl]-propyl}-isopropyl-carbamic acid tert-butyl ester

A solution of 8-(6-iodo-benzo[1,3]dioxol-5-ylmethyl)-9H-purin-6-ylamine previously prepared (cf. Chiosis G. et al., “Small-molecule HSP90 inhibitors, WO2006084030) ((0.9 g, 2.7 mmol), toluene-4-sulfonic acid 1-(tert-butoxycarbonyl-isopropyl-amino)-propyl ester previously prepared (cf. Llauger et al., “Evaluation of 8-Arylsulfanyl, 8-Arylsulfoxyl, and 8-Arylsulfonyl Adenine Derivatives as Inhibitors of the Heat Shock Protein 90”, J. Med. Chem. 2005, vol. 48, p. 2892) (1.26 g, 3.4 mmol), and Cs₂CO₃ (0.7 g, 2.2 mmol) in 24 mL of DMF was stirred at 80° C. for 16 h. Removal of the solvent and purification by chromatography on silica (CHCl₃/MeOH, 5%) yielded {3-[6-amino-8-(6-iodo-benzo[1,3]dioxol-5-ylmethyl)-purin-9-yl]-propyl}-isopropyl-carbamic acid tert-butyl ester (1.2 mg, 92%) as a viscous oil. ¹H-NMR [CDCl₃, δ, ppm]: 8.31 (s, 1H), 7.26 (s, 1H), 6.60 (s, 1H), 5.91 (s, 2H), 4.26 (s, 2H), 4.16 (m, 2H), 3.19 (m, 2H), 1.93 (m, 2H), 1.42 (s, 9H), 1.04 (d, 6H),

1.2 g (2.14 mmol) of {3-[6-amino-8-(6-iodo-benzo[1,3]dioxol-5-ylmethyl)-purin-9-yl]-propyl}-isopropyl-carbamic acid tert-butyl ester were dissolved in 58 mL of acetic acid. After 10 min stirring, a solution of 1.5 g (21.7 mmol) of NaNO₂ in 11 mL of H₂O was injected over the yellow solution. The brown mixture was stirred for 20 h at 25° C. The solvent was removed under reduced pressure and the crude was taken up in a mixture of EtOAc/H₂O. The aqueous phase was extracted twice and the combined organic phases were washed (NaHCO₃, brine) and dried (MgSO₄). Removal of the solvent under reduced pressure and a further purification by flash chromatography (CH₂Cl₂/EtOAc/MeOH, 100:100:3) yielded (200 mg, 16%) of {3-[8-(6-iodo-benzo[1,3]dioxol-5-ylmethyl)-6-oxo-1,6-dihydro-purin-9-yl]-propyl}-isopropyl-carbamic acid tert-butyl ester as a yellow foam. ¹H-NMR [CDCl₃, δ, ppm]: 8.31 (s, 1H), 7.28 (s, 1H), 6.74 (s, 1H), 5.94 (s, 2H), 4.31 (s, 2H), 4.18 (m, 2H), 3.21 (m, 2H), 1.90 (m, 2H), 1.42 (s, 9H), 1.05 (d, 6H).

### Example 43

### Preparation of {3-[1-(2,4-dinitro-phenyl)-8-(6-iodo-benzol[1,3]dioxol-5-ylmethyl)-6-oxo-1,6-dihydro-purin-9-yl]-propyl}-isopropyl-carbamic acid tert-butyl ester

{3-[8-(6-iodo-benzo[1,3]dioxol-5-ylmethyl)-6-oxo-1,6-dihydro-purin-9-yl]-propyl}-isopropyl-carbamic acid tert-butyl ester (0.15 g, 0.25 mmol), K₂CO₃ (87 mg, 0.63 mmol) and 1-chloro-2,4-dinitrobenzene (0.12 g, 0.63 mmol) were dissolved in 5 mL of distilled DMF. The resulting suspension was stirred at 85° C. for 18 h. After removing the solvent under reduced pressure, the crude was purified by flash chromatography (CHCl₃ 100%). {3-[1-(2,4-dinitro-phenyl)-8-(6-iodo-benzo[1,3]dioxol-5-ylmethyl)-6-oxo-1,6-dihydro-purin-9-yl]-propyl}-isopropyl-carbamic acid tert-butyl ester (40 mg, 21%) as a foam were obtained. ¹H-NMR [CDCl₃, δ, ppm]: 9.10 (s, 1H), 8.56 (s, 1H), 8.56 (s, 1H), 8.18 (s, 1H), 7.98 (s, 1H), 6.59 (s, 1H), 5.97 (s, 2H), 4.12 (s, 2H), 4.09 (m, 2H), 3.12 (m, 2H), 1.07 (d, 6H), 1.97 (m, 2H), 1.52 (s, 9H).

### Example 44

### Preparation of {3-[5-amino-4-carbamoyl-2-(6-iodo-benzol[1,3]dioxol-5-ylmethyl)-imidazol-1-yl]-propyl}-isopropyl-carbamic acid tert-butyl ester

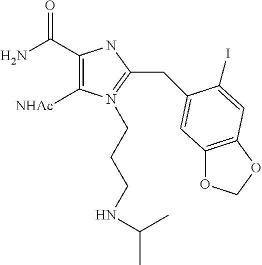

2 mL of ethylenediamine were injected over 40 mg (0.05 mmol) of {3-[1-(2,4-dinitro-phenyl)-8-(6-iodo-benzo[1,3]dioxol-5-ylmethyl)-6-oxo-1,6-dihydro-purin-9-yl]-propyl}-isopropyl-carbamic acid tert-butyl ester and the immediate formed dark red solution was stirred at 50° C. for 6 h. Solvent was removed under reduced pressure and co evaporated with toluene. The resulting product was isolated by flash column chromatography (CHCl₃:MeOH 3%), yielding (25 mg, 83%) of a brown oil as the desired product 3-[5-amino-4-carbamoyl-2-(6-iodo-benzo[1,3]dioxol-5-ylmethyl)-imidazol-1-yl]-propyl}-isopropyl-carbamic acid tert-butyl ester. ¹H-NMR [CDCl₃, δ, ppm]: 7.48 (s, 1H), 6.55 (s, 1H), 5.99 (s, 2H), 4.12 (s, 2H), 3.68 (m, 2H), 3.12 (m, 2H), 1.95 (m, 2H), 1.48 (s, 9H), 1.04 (d, 6H).

### Example 45

### Preparation of 5-amino-2-(6-iodo-benzol[1,3]dioxol-5-ylmethyl)-1-(3-isopropylamino-propyl)-1H-imidazole-4-carboxamide

0.15 mL f TFA was added to a solution of 3-[5-amino-4-carbamoyl-2-(6-iodo-benzo[1,3]dioxol-5-ylmethyl)-imidazol-1-yl]-propyl}-isopropyl-carbamic acid tert-butyl ester (15 mg, 0.025 mmol) in 0.5 mL of CH₂Cl₂. This reaction mixture was stirred at room temperature for 16 h. Removal of the solvent and purification by chromatography on silica (CHCl₃/MeOH/NH₄OH, 90:10:2) yielded 5-amino-2-(6-iodo-benzo[1,3]dioxol-5-ylmethyl)-1-(3-isopropylamino-propyl)-1H-imidazole-4-carboxamide (0.19 mg, 2%) as a viscous oil. ¹H-NMR [CDCl₃, δ, ppm]: 7.48 (s, 1H), 6.65 (s, 1H), 6.01 (s, 2H), 4.12 (s, 2H), 3.98 (m, 2H), 3.12 (m, 2H), 2.01 (m, 2H) 1.22 (d, 6H). MS (EI, m/z) 486 (M⁺+1).

### Example 46

### Preparation of 5-amino-1-butyl-1H-imidazole-4-carboxamide

A solution of 5-amino-1H-imidazole-4-carboxamide (0.6 g, 4.7 mmol), toluene-4-sulfonic acid butyl ester previously prepared (cf. Sekera, V. et al., “Higher alkyl sulfonates”, J. Am. Chem. Soc., 1993, vol. 55, p. 345) (1.3 g, 5.7 mmol), and Cs₂CO₃ (1.5 g, 4.7 mmol) in 25 mL of DMF was stirred at 80° C. for 16 h. Removal of the solvent and purification by chromatography on silica (CHCl₃/MeOH, 5%) yielded 5-amino-1-butyl-1H-imidazole-4-carboxamide (0.5 g, 62%) as a viscous oil. ¹H-NMR [CDCl₃, δ, ppm]: 8.1 (s, 1H), 3.79 (m, 2H, CH₂N), 1.54 (m, 2H, CH₂), 1.37 (m, 2H, CH₂), 0.92 (m, 3H, CH₃).

### Example 47

### Preparation of 5-amino-2-bromo-1-butyl-1H-imidazole-4-carboxamide

A solution of 5-amino-1-butyl-1H-imidazole-4-carboxamide (44 mg, 0.2 mmol) and N-bromosuccinimide (47 mg, 4.7 mmol) in 3 mL of CHCl₃ was stirred at reflux for 20 minutes. Removal of the solvent and purification by chromatography on silica (CHCl₃/MeOH, 10%) yielded 5-amino-2-bromo-1-butyl-1H-imidazole-4-carboxamide (29 mg, 46%) as a viscous oil. ¹H-NMR [CDCl₃, δ, ppm]: 3.79 (m, 2H, CH₂N), 1.54 (m, 2H, CH₂), 1.37 (m, 2H, CH₂), 0.92 (m, 3H, CH₃).

### Example 48

### Preparation of 5-amino-1-butyl-2-(7-chloro-benzothiazol-2-ylsulfanyl)-1H-imidazole-4-carboxamide

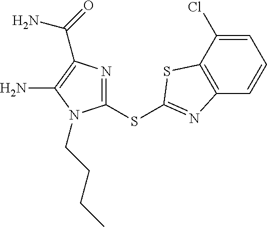

A solution of 5-amino-2-bromo-1-butyl-1H-imidazole-4-carboxamide (46 mg, 0.17 mmol), potassium tert-butoxide (64 mg, 0.52 mmol), lithium bromide (76 mg, 0.8 mmol) and 7-chloro-benzothiazole-2-thiol (86 mg, 0.4 mmol) in 7 mL of DMF was stirred at 120° C. for 16 h. Removal of the solvent and purification by chromatography on silica (CHCl₃/MeOH, 10%) yielded 5-amino-1-butyl-2-(7-chloro-benzothiazol-2-ylsulfanyl)-1H-imidazole-4-carboxamide (33 mg, 49%) as a foam. ¹H-NMR [CDCl₃, δ, ppm]: 7.73 (m, 1H), 7.33 (m, 1H), 7.26 (m, 1H), 3.89 (m, 2H, CH₂N), 1.54 (m, 2H, CH₂), 1.37 (m, 2H, CH₂), 0.92 (m, 3H, CH₃). MS (EI, m/z) 382 (M⁺+1).

### Example 49

### Preparation of 5-amino-1-butyl-2-(4-phenyl-thiazol-2-ylsulfanyl)-1H-imidazole-4-carboxamide

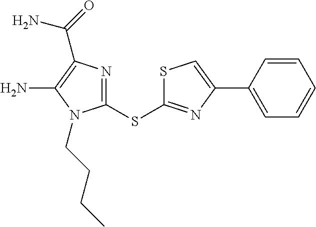

A solution of 5-amino-2-bromo-1-butyl-1H-imidazole-4-carboxamide (30 mg, 0.11 mmol), potassium tert-butoxide (31 mg, 0.25 mmol), lithium bromide (50 mg, 0.5 mmol) and 4-phenyl-thiazole-2-thiol (41 mg, 0.2 mmol) in 7 mL of DMF was stirred at 120° C. for 16 h. Removal of the solvent and purification by chromatography on silica (CHCl₃/MeOH, 10%) yielded 5-amino-1-butyl-2-(4-phenyl-thiazol-2-ylsulfanyl)-1H-imidazole-4-carboxamide (41 mg, 0.2 mmol) (8 mg, 18%) as a foam. ¹H-NMR [CDCl₃, δ, ppm]: 7.79 (m, 1H), 7.39-7.38 (m, 5H), 3.89 (m, 2H, CH₂N), 1.54 (m, 2H, CH₂), 1.37 (m, 2H, CH₂), 0.92 (m, 3H, CH₃). MS (EI, m/z) 374 (M⁺+1).

### Example 50

### Preparation of 5-amino-2-bromo-1-methyl-1H-imidazole-4-carboxamide

N-bromosuccinimide (0.3 g, 1.57 mmol) was added to a solution of 5-amino-1-methyl-1H-imidazole-4-carboxamide (0.2 g, 1.42 mmol) in DMF (10 mL). The reaction mixture was stirred at room temperature for 15 min. Solvent was removed and purification of the crude by flash column chromatography (CH₂Cl₂/MeOH 92:8) afforded 5-amino-2-bromo-1-methyl-1H-imidazole-4-carboxamide (60 mg, 19%) as foam. ¹H-NMR [CD₃OD, δ, ppm]: 3.44 (s, 3H, CH₃).

### Example 51

### Preparation of 5-amino-2-(7-chloro-benzothiazol-2-ylsulfanyl)-1-methyl-1H-imidazole-4-carboxamide

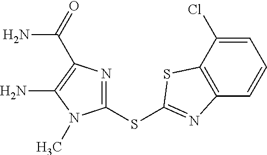

In a 3-necked round bottomed flask, containing a magnetic stirrer, 80 mg (0.36 mmol) of 5-amino-2-bromo-1-methyl-1H-imidazole-4-carboxamide, 0.16 g (1.83 mmol) of LiBr, 98 mg (0.80 mmol) of potassium tert-butoxide and 8.85 g (64 mmol) of 7-chloro-benzothiazole-2-thiol were weighted. The flask was purged with argon and 6 ml of distilled DMF were added by syringe. The resulting suspension was stirred overnight at 130° C. After this, the solvent was removed under high vacuum and the crude was purified through flash chromatography (SiO₂, CH₂Cl₂/MeOH:96/4 affording the 5-amino-2-(7-chloro-benzothiazol-2-ylsulfanyl)-1-methyl-1H-imidazole-4-carboxamide compound (38 mg, 31%) as a foam. ¹H-NMR [CD₃OD, δ, ppm]: 7.81 (m, 1H), 7.48 (m, 1H), 7.42 (m, 1H), 3.56 (s, 3H, CH₃). MS (EI, m/z) 340 (M⁺+1).

### Example 52

### Preparation of [3-(5-amino-4-carbamoyl-imidazol-1-yl)-propyl]-isopropyl-carbamic acid tert-butyl ester

A solution of 5-amino-1H-imidazole-4-carboxamide (1.4 g, 11 mmol), toluene-4-sulfonic acid 1-(tert-butoxycarbonyl-isopropyl-amino)-propyl ester (5 g, 13 mmol) previously prepared (cf. Llauger et al., “Evaluation of 8-Arylsulfanyl, 8-Arylsulfoxyl, and 8-Arylsulfonyl Adenine Derivatives as Inhibitors of the Heat Shock Protein 90”, J. Med. Chem. 2005, vol. 48, p. 2892), and Cs₂CO₃ (3.6 g, 11 mmol) in 40 mL of DMF was stirred at 80° C. for 16 h. Removal of the solvent and purification by chromatography on silica (CHCl₃/MeOH, 5%) yielded [3-(5-Amino-4-carbamoyl-imidazol-1-yl)-propyl]-isopropyl-carbamic acid tert-butyl ester (1.45 g, 40%) as a viscous oil. ¹H-NMR [CDCl₃, δ, ppm]: 7.12 (s, 1H), 3.37 (m, 2H), 3.32 (m, 1H), 3.07 (m, 2H), 1.92 (m, 2H), 1.40 (s, 9H), 1.07-1.06 (2s, 6H).

### Example 53

### Preparation of [3-(5-amino-2-bromo-4-carbamoyl-imidazol-1-yl)-propyl]-isopropyl-carbamic acid tert-butyl ester

N-bromosuccinimide (0.9 g, 4.9 mmol) was added to a solution of [3-(5-amino-4-carbamoyl-imidazol-1-yl)-propyl]-isopropyl-carbamic acid tert-butyl ester (1.4 g, 4.4 mmol) in DMF (80 mL). The reaction mixture was stirred at room temperature for 15 min. Solvent was removed and purification of the crude by flash column chromatography (CH₂Cl₂/MeOH 92:8) afforded the [3-(5-amino-2-bromo-4-carbamoyl-imidazol-1-yl)-propyl]-isopropyl-carbamic acid tert-butyl ester (1.2 mg, 68%) compound as foam. ¹H-NMR [CDCl₃, δ, ppm]: 3.37 (m, 2H), 3.32 (m, 1H), 3.07 (m, 2H), 1.92 (m, 2H), 1.40 (s, 9H), 1.07-1.06 (2s, 6H).

### Example 54

### Preparation of {3-[5-amino-4-carbamoyl-2-(7-chloro-benzothiazol-2-ylsulfanyl)-imidazol-1-yl]-propyl}-isopropyl-carbamic acid tert-butyl ester

In a 3-necked round bottomed flask, containing a magnetic stirrer, 32 mg (0.7 mmol) of [3-(5-amino-2-bromo-4-carbamoyl-imidazol-1-yl)-propyl]-isopropyl-carbamic acid tert-butyl ester, 34 mg (0.4 mmol) of LiBr, 22 mg (0.17 mmol) of potassium tert-butoxide and 28 mg (0.14 mmol) of 7-chloro-benzothiazole-2-thiol were weighted. The flask was purged with argon and 6 mL of distilled DMF were added by syringe. The resulting suspension was stirred overnight at 130° C. After this, the solvent was removed under high vacuum and the crude was purified through flash chromatography (SiO₂, CH₂Cl₂/MeOH: 96/4) affording the 3-[5-amino-4-carbamoyl-2-(7-chloro-benzothiazol-2-ylsulfanyl)-imidazol-1-yl]-propyl}-isopropyl-carbamic acid tert-butyl ester compound (14 mg, 34%) as a foam. ¹H-NMR [CD₃OD, δ, ppm]: 7.77 (m, 1H), 7.38 (m, 1H), 7.28 (m, 1H), 4.00 (m, 2H), 3.10 (m, 1H), 1.90 (m, 4H), 1.42 (s, 9H), 1.01-1.04 (2s, 6H).

### Example 55

### Preparation of 5-amino-2-(7-chloro-benzothiazol-2-ylsulfanyl)-1-(3-isopropylamino-propyl)-1H-imidazole-4-carboxamide

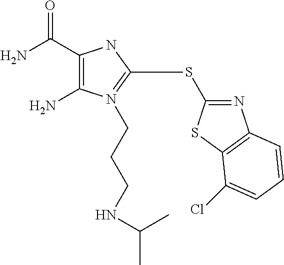

TFA (0.2 mL) was added to a solution of 3-[5-amino-4-carbamoyl-2-(7-chloro-benzothiazol-2-ylsulfanyl)-imidazol-1-yl]-propyl}-isopropyl-carbamic acid tert-butyl ester (14 mg, 0.26 mmol) in 2 mL of CH₂Cl₂. This reaction mixture was stirred at room temperature for 16 h. Removal of the solvent and purification by chromatography on silica (CHCl₃/MeOH/NH₄OH, 90:10:2) yielded 5-amino-2-(7-chloro-benzothiazol-2-ylsulfanyl)-1-(3-isopropylamino-propyl)-1H-imidazole-4-carboxamide (10 mg, 92%) as a viscous oil. ¹H-NMR [CDCl₃, δ, ppm]: 7.70 (m, H), 7.33 (m, 1H), 7.24 (m, 1H), 3.98 (m, 2H), 2.27 (m, 1H), 2.48 (m, 2H), 1.78 (m, 2H), 1.0-0.90 (2s, 6H). MS (EI, m/z) 425 (M⁺+1).

### Example 56

### Preparation of {3-[5-amino-4-carbamoyl-2-(1-isopropyl-1H-benzoimidazol-2-ylsulfanyl)-imidazol-1-yl]-propyl}-isopropyl-carbamic acid tert-butyl ester

In a 3-necked round bottomed flask, containing a magnetic stirrer, 0.3 g (0.7 mmol) of [3-(5-amino-2-bromo-4-carbamoyl-imidazol-1-yl)-propyl]-isopropyl-carbamic acid tert-butyl ester, 0.3 g (3.6 mmol) of LiBr, 0.19 g (1.7 mmol) of potassium tert-butoxide and 0.25 g (1.32 mmol) of 1-isopropyl-1H-benzoimidazole-2-thiol were weighted. The flask was purged with argon and 6 mL of distilled DMF were added by syringe. The resulting suspension was stirred overnight at 130° C. After this, the solvent was removed under high vacuum and the crude was purified through flash chromatography (SiO₂, CH₂Cl₂/MeOH:96/4) affording the {3-[5-amino-4-carbamoyl-2-(1-isopropyl-1-benzoimidazol-2-ylsulfanyl)-imidazol-1-yl]-propyl}-isopropyl-carbamic acid tert-butyl ester (70 mg, 18%) compound as a foam. ¹H-NMR [CD₃OD, δ, ppm]: 7.70 (m, 1H), 7.56 (m, 1H), 7.26 (m, 2H), 4.00 (m, 2H), 3.10 (m, 1H), 2.98 (m, 1H), 1.90 (m, 4H), 1.59 (2s, 6H), 1.42 (s, 9H), 1.01-1.04 (2s, 6H).

### Example 57

### Preparation of 5-amino-1-(3-isopropylamino-propyl)-2-(1-isopropyl-1H-benzoimidazol-2-ylsulfanyl)-1H-imidazole-4-carboxamide

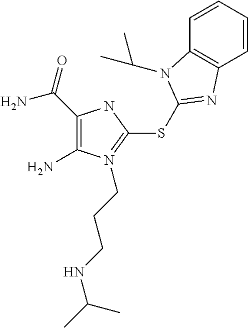

TFA (0.5 mL) was added to a solution of {3-[5-amino-4-carbamoyl-2-(1-isopropyl-1H-benzoimidazol-2-ylsulfanyl)-imidazol-1-yl]-propyl}-isopropyl-carbamic acid tert-butyl ester (70 mg, 0.13 mmol) in 7 mL of CH₂Cl₂. This reaction mixture was stirred at room temperature for 16 h. Removal of the solvent and purification by chromatography on silica (CHCl₃/MeOH/NH₄OH, 90:10:2) yielded 5-amino-1-(3-isopropylamino-propyl)-2-(1-isopropyl-1H-benzoimidazol-2-ylsulfanyl)-1H-imidazole-4-carboxamide (52 mg, 92%) as a viscous oil. ¹H-NMR [CDCl₃, δ, ppm]: 7.70 (m, 1H), 7.56 (m, 1H), 7.26 (m, 2H), 4.00 (m, 2H), 3.10 (m, 1H), 2.98 (m, 1H), 1.90 (m, 4H), 1.59 (2s, 6H), 1.01-1.04 (2s, 6H). MS (EI, m/z) 416 (M⁺+1).

### Example 58

### Preparation of {3-[5-amino-4-carbamoyl-2-(3-chloro-5-trifluoromethyl-pyridin-2-ylsulfanyl)-imidazol-1-yl]-propyl}-isopropyl-carbamic acid tert-butyl ester

In a 3-necked round bottomed flask, containing a magnetic stirrer, 0.1 g (0.25 mmol) of [3-(5-amino-2-bromo-4-carbamoyl-imidazol-1-yl)-propyl]-isopropyl-carbamic acid tert-butyl ester, 0.1 g (1.2 mmol) of LiBr, 0.07 g (0.5 mmol) of potassium tert-butoxide and 0.1 g (0.45 mmol) of 3-Chloro-5-trifluoromethyl-pyridine-2-thiol were weighted. The flask was purged with argon and 2 mL of distilled DMF were added by syringe. The resulting suspension was stirred overnight at 130° C. After this, the solvent was removed under high vacuum and the crude was purified through flash chromatography (SiO₂, CH₂Cl₂/MeOH:96/4 affording the desired compound {3-[5-amino-4-carbamoyl-2-(3-chloro-5-trifluoromethyl-pyridin-2-ylsulfanyl)-imidazol-1-yl]-propyl}-isopropyl-carbamic acid tert-butyl ester (18 mg, 13%) as a foam. ¹H-NMR [CD₃OD, δ, ppm]: 8.42 (m, 1H), 7.78 (m, 1H), 3.79 (m, 2H), 3.10 (m, 1H), 1.90 (m, 4H), 1.42 (s, 9H), 1.01-1.04 (2s, 6H).

### Example 59

### Preparation of 5-amino-2-(3-chloro-5-trifluoromethyl-pyridin-2-ylsulfanyl)-1-(3-isopropylamino-propyl)-1H-imidazole-4-carboxamide

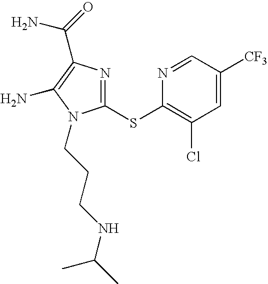

TFA (0.1 mL) was added to a solution of {3-[5-amino-4-carbamoyl-2-(3-chloro-5-trifluoromethyl-pyridin-2-ylsulfanyl)-imidazol-1-yl]-propyl}-isopropyl-carbamic acid tert-butyl ester (15 mg, 0.03 mmol) in 1 mL of CH₂Cl₂. This reaction mixture was stirred at room temperature for 16 h. Removal of the solvent and purification by chromatography on silica (CHCl₃/MeOH/NH₄OH, 90:10:2) yielded 5-amino-2-(3-chloro-5-trifluoromethyl-pyridin-2-ylsulfanyl)-1-(3-isopropylamino-propyl)-1H-imidazole-4-carboxamide (7.5 mg, 62%) as a viscous oil. ¹H-NMR [CD₃OD, δ, ppm]: 8.42 (m, 1H), 7.78 (m, 1H), 3.79 (m, 2H), 3.10 (m, 1H), 1.90 (m, 4H), 1.01-1.04 (2s, 6H). MS (EI, m/z) 437 (M⁺+1).

### Example 60

### Preparation of {3-[5-amino-4-carbamoyl-2-(naphthalen-2-ylsulfanyl)-imidazol-1-yl]-propyl}-isopropyl-carbamic acid tert-butyl ester

In a 3-necked round bottomed flask, containing a magnetic stirrer, 0.1 g (0.25 mmol) of [3-(5-amino-2-bromo-4-carbamoyl-imidazol-1-yl)-propyl]-isopropyl-carbamic acid tert-butyl ester, 0.1 g (1.2 mmol) of LiBr, 0.06 g (0.5 mmol) of potassium tert-butoxide and 0.07 g (0.4 mmol) of naphthalene-2-thiol were weighted. The flask was purged with argon and 2 mL of distilled DMF were added by syringe. The resulting suspension was stirred overnight at 130° C. After this, the solvent was removed under high vacuum and the crude was purified through flash chromatography (SiO₂, CH₂Cl₂/MeOH:96/4, affording the compound {3-[5-amino-4-carbamoyl-2-(naphthalen-2-ylsulfanyl)-imidazol-1-yl]-propyl}-isopropyl-carbamic acid tert-butyl ester (70 mg, 58%) as a foam. ¹H-NMR [CD₃OD, δ, ppm]: 7.78 (m, 3H), 7.60 (m, 1H), 7.44 (m, 2H), 7.27 (m, 1H), 3.79 (m, 2H), 3.10 (m, 1H), 1.90 (m, 4H), 1.42 (s, 9H), 1.01-1.04 (2s, 6H).

### Example 61

### Preparation of 5-amino-1-(3-isopropylamino-propyl)-2-(naphthalen-2-ylsulfanyl)-1H-imidazole-4-carboxamide

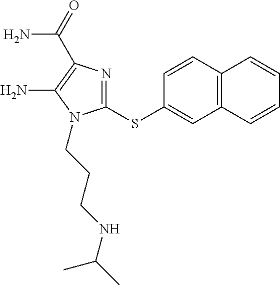

TFA (0.1 mL) was added to a solution of 3-[5-amino-4-carbamoyl-2-(naphthalen-2-ylsulfanyl)-imidazol-1-yl]-propyl}-isopropyl-carbamic acid tert-butyl ester (22 mg, 0.04 mmol) in 1 mL of CH₂Cl₂. This reaction mixture was stirred at room temperature for 16 h. Removal of the solvent and purification by chromatography on silica (CHCl₃/MeOH/NH₄OH, 90:10:2) yielded -amino-1-(3-isopropylamino-propyl)-2-(naphthalen-2-ylsulfanyl)-1H-imidazole-4-carboxamide (17 mg, 97%) as a viscous oil. ¹H-NMR [CD₃OD, δ, ppm]: 7.78 (m, 3H), 7.60 (m, 1H), 7.44 (m, 2H), 7.27 (m, 1H), 3.79 (m, 2H), 3.10 (m, 1H), 1.90 (m, 4H), 1.01-1.04 (2s, 6H). MS (EI, m/z) 384 (M⁺+1).

### Example 62

### Preparation of {3-[5-amino-4-carbamoyl-2-(4-phenyl-thiazol-2-ylsulfanyl)-imidazol-1-yl]-propyl}-isopropyl-carbamic acid tert-butyl ester

In a 3-necked round bottomed flask, containing a magnetic stirrer, 0.1 g (0.25 mmol) of [3-(5-amino-2-bromo-4-carbamoyl-imidazol-1-yl)-propyl]-isopropyl-carbamic acid tert-butyl ester, 0.1 g (1.2 mmol) of LiBr, 0.06 g (0.5 mmol) of potassium tert-butoxide and 0.09 g (0.4 mmol) of 4-Phenyl-thiazole-2-thiol were weighted. The flask was purged with argon and 2 mL of distilled DMF were added by syringe. The resulting suspension was stirred overnight at 130° C. After this, the solvent was removed under high vacuum and the crude was purified through flash chromatography (SiO₂, CH₂Cl₂/MeOH:96/4) affording the compound 3-[5-amino-4-carbamoyl-2-(4-phenyl-thiazol-2-ylsulfanyl)-imidazol-1-yl]-propyl}-isopropyl-carbamic acid tert-butyl ester (64 mg, 39%) as a foam. ¹H-NMR [CD₃OD, δ, ppm]: 7.98 (m, 1H), 7.80 (m, 2H), 7.35 (m, 3H), 7.27 (m, 1H), 3.79 (m, 2H), 3.10 (m, 1H), 1.90 (m, 4H), 1.42 (s, 9H), 1.01-1.04 (2s, 6H).

### Example 63

### Preparation of 5-amino-1-(3-isopropylamino-propyl)-2-(4-phenyl-thiazol-2-ylsulfanyl)-1H-imidazole-4-carboxamide

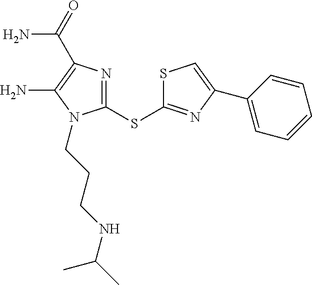

TFA (0.1 mL) was added to a solution of 3-[5-amino-4-carbamoyl-2-(4-phenyl-thiazol-2-ylsulfanyl)-imidazol-1-yl]-propyl}-isopropyl-carbamic acid tert-butyl ester (64 mg, 0.12 mmol) in 1 mL of CH₂Cl₂. This reaction mixture was stirred at room temperature for 16 h. Removal of the solvent and purification by chromatography on silica (CHCl₃/MeOH/NH₄OH, 90:10:2) yielded 5-amino-1-(3-isopropylamino-propyl)-2-(4-phenyl-thiazol-2-ylsulfanyl)-1H-imidazole-4-carboxamide (8 mg, 92%) as a viscous oil. ¹H-NMR [CD₃OD, δ, ppm]: 7.98 (m, 1H), 7.80 (m, 2H), 7.35 (m, 3H), 7.27 (m, 1H), 3.79 (m, 2H), 3.10 (m, 1H), 1.90 (m, 4H), 1.01-1.04 (2s, 6H). MS (EI, m/z) 417 (M⁺+1).

### Example 64

### Preparation of {3-[4-carbamoyl-5-(cyclopropanecarbonyl-amino)-2-(6-iodo-benzol[1,3]dioxol-5-ylsulfanyl)-imidazol-1-yl]-propyl}-isopropyl-carbamic acid tert-butyl ester

Cyclopropanecarbonyl chloride (7 μL, 0.08 mmol) and diisopropylethylamine (28 μL, 0.16 mmol were added to a solution of {3-[5-amino-4-carbamoyl-2-(6-iodo-benzo[1,3]dioxol-5-ylsulfanyl)-imidazol-1-yl]-propyl}-isopropyl-carbamic acid tert-butyl ester (24 mg, 0.05 mmol) in CH₂Cl₂ (2 mL). The reaction mixture was stirred for 16 h at room temperature. Solvent was removed and purification of the crude by flash column chromatography (CH₂Cl₂/MeOH 98:2) afforded the desired product {3-[4-carbamoyl-5-(cyclopropanecarbonyl-amino)-2-(6-iodo-benzo[1,3]dioxol-5-ylsulfanyl)-imidazol-1-yl]-propyl}-isopropyl-carbamic acid tert-butyl ester (7.3 mg, 27%) as colourless oil. ¹H-NMR [CDCl₃, δ, ppm]: 7.20 (s, 1H), 6.53 (s, 1H), 6.82 (d, J=8.0 Hz, 1H), 5.92 (s, 2H, OCH₂O), 4.48 (t, J=7.5 Hz, 2H, CH₂N), 3.10 (m, 1H), 1.90 (m, 4H), 1.42 (s, 9H), 1.14 (m, CH₂ cyc), 1.01-1.04 (2s, 6H), 0.84 (m, CH₂ cyc).

### Example 65

### Preparation of 5-(cyclopropanecarbonyl-amino)-2-(6-iodo-benzo[1,3]dioxol-5-ylsulfanyl)-1-(3-isopropylamino-propyl)-1H-imidazole-4-carboxamide

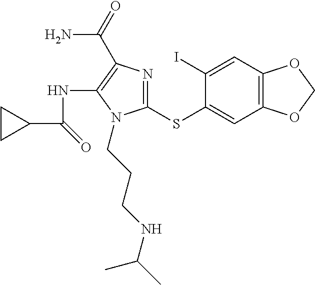

TFA (0.1 mL) was added to a solution of {3-[4-carbamoyl-5-(cyclopropane carbonyl-amino)-2-(6-iodo-benzo[1,3]dioxol-5-ylsulfanyl)-imidazol-1-yl]-propyl}-isopropyl-carbamic acid tert-butyl ester (7.3 mg, 0.01 mmol) in 1 mL of CH₂Cl₂. This reaction mixture was stirred at room temperature for 16 h. Removal of the solvent and purification by chromatography on silica (CHCl₃/MeOH/NH₄OH, 90:10:2) yielded 5-(cyclopropanecarbonyl-amino)-2-(6-iodo-benzo[1,3]dioxol-5-ylsulfanyl)-1-(3-isopropylamino-propyl)-1H-imidazole-4-carboxamide (5.6 mg, 90%) as a viscous oil. ¹H-NMR [CD₃OD, δ, ppm]: 7.20 (s, 1H), 6.53 (s, 1H), 6.82 (d, J=8.0 Hz, 1H), 5.92 (s, 2H, OCH₂O), 4.48 (t, J=7.5 Hz, 2H, CH₂N), 3.10 (m, 1H), 1.90 (m, 4H), 1.14 (m, CH₂ cyc), 1.01-1.04 (2s, 6H), 0.84 (m, CH₂ cyc). MS (EI, m/z) 572 (M⁺+1).

### Example 66

### Preparation of {3-[5-acetylamino-4-carbamoyl-2-(7-chloro-benzothiazol-2-ylsulfanyl)-imidazol-1-yl]-propyl}-isopropyl-carbamic acid tert-butyl ester

Acetyl chloride (19 μL, 0.2 mmol) and diisopropylethylamine (89 μL, 0.5 mmol were added to a solution of {3-[5-amino-4-carbamoyl-2-(7-chloro-benzothiazol-2-ylsulfanyl)-imidazol-1-yl]-propyl}-isopropyl-carbamic acid tert-butyl ester (67 mg, 0.1 mmol) in CH₂Cl₂ (6 mL). The reaction mixture was stirred for 16 h at room temperature. Solvent was removed and purification of the crude by flash column chromatography (CH₂Cl₂/MeOH 98:2) afforded the desired product 3-[5-acetylamino-4-carbamoyl-2-(7-chloro-benzothiazol-2-ylsulfanyl)-imidazol-1-yl]-propyl}-isopropyl-carbamic acid tert-butyl ester (10 mg, 14%) as colourless oil. ¹H-NMR [CDCl₃, δ, ppm]: 7.77 (m, 1H), 7.38 (m, 1H), 7.28 (m, 1H), 4.00 (m, 2H), 3.10 (m, 1H), 2.25 (s, 3H, OCH₃), 1.90 (m, 4H), 1.42 (s, 9H), 1.01-1.04 (2s, 6H).

### Example 67

### Preparation of 5-acetylamino-2-(7-chloro-benzothiazol-2-ylsulfanyl)-1-(3-isopropylamino-propyl)-1H-imidazole-4-carboxamide

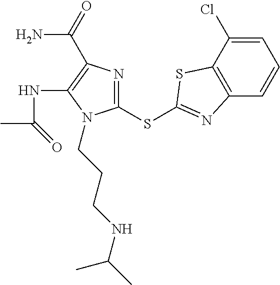

TFA (0.1 mL) was added to a solution of 3-[5-acetylamino-4-carbamoyl-2-(7-chloro-benzothiazol-2-ylsulfanyl)-imidazol-1-yl]-propyl}-isopropyl-carbamic acid tert-butyl ester (8 mg, 0.01 mmol) in 1 mL of CH₂Cl₂. This reaction mixture was stirred at room temperature for 16 h. Removal of the solvent and purification by chromatography on silica (CHCl₃/MeOH/NH₄OH, 90:10:2) yielded 5-Acetylamino-2-(7-chloro-benzothiazol-2-ylsulfanyl)-1-(3-isopropylamino-propyl)-1H-imidazole-4-carboxamide (4 mg, 57%) as a viscous oil. ¹H-NMR [CD₃OD, δ, ppm]: 7.77 (m, 1H), 7.38 (m, 1H), 7.28 (m, 1H), 4.00 (m, 2H), 3.10 (m, 1H), 2.25 (s, 3H, OCH₃), 1.90 (m, 4H), 1.01-1.04 (2s, 6H). MS (EI, m/z) 468 (M⁺+1).

### Example 68

### Preparation of {3-[5-acetylamino-4-carbamoyl-2-(6-iodo-benzo[1,3]dioxol-5-ylsulfanyl)-imidazol-1-yl]-propyl}-isopropyl-carbamic acid tert-butyl ester

Acetyl chloride (5 μL, 0.07 mmol) and diisopropylethylamine (26 μL, 0.15 mmol were added to a solution of {3-[5-amino-4-carbamoyl-2-(6-iodo-benzo[1,3]dioxol-5-ylsulfanyl)-imidazol-1-yl]-propyl}-isopropyl-carbamic acid tert-butyl ester (22 mg, 0.03 mmol) in CH₂Cl₂ (2 mL). The reaction mixture was stirred for 16 h at room temperature. Solvent was removed and purification of the crude by flash column chromatography (CH₂Cl₂/MeOH 98:2) afforded the desired product 3-[5-acetylamino-4-carbamoyl-2-(6-iodo-benzo[1,3]dioxol-5-ylsulfanyl)-imidazol-1-yl]-propyl}-isopropyl-carbamic acid tert-butyl ester (9 mg, 38%) as colourless oil. ¹H-NMR [CDCl₃, δ, ppm]: 7.20 (s, 1H), 6.53 (s, 1H), 6.82 (d, J=8.0 Hz, 1H), 5.92 (s, 2H, OCH₂O), 4.48 (t, J=7.5 Hz, 2H, CH₂N), 3.10 (m, 1H), 2.25 (s, 3H, OCH₃), 1.90 (m, 4H), 1.42 (s, 9H), 1.01-1.04 (2s, 6H).

### Example 69

### Preparation of 5-acetylamino-2-(6-iodo-benzo[1,3]dioxol-5-ylsulfanyl)-1-(3-isopropylamino-propyl)-1H-imidazole-4-carboxamide

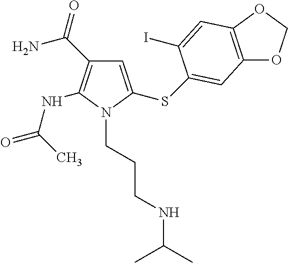

TFA (0.1 mL) was added to a solution of 3-[5-acetylamino-4-carbamoyl-2-(6-iodo-benzo[1,3]dioxol-5-ylsulfanyl)-imidazol-1-yl]-propyl}-isopropyl-carbamic acid tert-butyl ester (9 mg, 0.01 mmol) in 1 mL of CH₂Cl₂. This reaction mixture was stirred at room temperature for 16 h. Removal of the solvent and purification by chromatography on silica (CHCl₃/MeOH/NH₄OH, 90:10:2) yielded 5-acetylamino-2-(6-iodo-benzo[1,3]dioxol-5-ylsulfanyl)-1-(3-isopropylamino-propyl)-1H-imidazole-4-carboxamide (6 mg, 79%) as a viscous oil. ¹H-NMR [CD₃OD, δ, ppm]: 7.20 (s, 1H), 6.53 (s, 1H), 6.82 (d, J=8.0 Hz, 1H), 5.92 (s, 2H, OCH₂O), 4.48 (t, J=7.5 Hz, 2H, CH₂N), 3.10 (m, 1H), 2.25 (s, 3H, OCH₃), 1.90 (m, 4H), 1.01-1.04 (2s, 6H). MS (EI, m/z) 546 (M⁺+1)

### Example 70

### Preparation of 5-amino-2-(6-iodo-benzol[1,3]dioxol-5-ylsulfanyl)-1-methyl-1H-imidazole-4-carboxamide

A mixture of 4-iodo-1,2-(methylenedioxy)benzene (4 g, 16.1 mmol), iodine (2 g, 7.9 mmol), ethanol (10 ml) and sulphuric acid (1 ml) was heated to 70° C. followed by the addition of hydrogen peroxide (5 ml, 30%) over the period of 15 min. Reaction mixture was stirred at 70° C. for additional 15 min and then cooled to the room temperature. Reaction mixture was extracted with CHCl₃ (50 ml), washed with water, 5% Na₂SO₃, brine and dried over anhydrous MgSO₄. Volatiles were removed under reduced pressure. Residue was purified by flash chromatography (CombiFlash® Companion unit equipped with RediSep® flash column (normal phase, 35-60 micron average particle size silicagel, 40 g, Teledyne Isco); flow rate=35 mL/min; injection volume 2 mL; mobile phase A: hexane; mobile phase B: EtOAc; gradient 0-70% B in 1 h.) Fractions containing the desired product were combined and concentrated in vacuum to provide target product (1 g, yield: 18%)) of 4,5-diiodo-1,2-(methylenedioxy)benzene as white crystals.

A mixture of 4,5-diiodo-1,2-(methylenedioxy)benzene (700 mg, 88 mmol), 5-amino-2-mercapto-1-methyl-1H-imidazole-4-carboxamide (ChemBridge, 350 mg, 2 mmol), [Cu(neocup)(PPh₃)Br] (100 mg) 1M K₂CO₃ (6 ml) and 12 mL MeCN was irradiated under microwave conditions 10 minutes at 120° C. (Biotage, Initiator™ Sixty). Reaction mixture was submitter for HPLC purification. (Varian L/L 4002-2 column (5×50 cm; Microsorb 100-10 C-18), flow rate=50 mL/min; mobile phase A: 100% water, 0.1% TFA; mobile phase B: 100% ACN, 0.1% TFA; gradient elution: from 0% B to 90% B in 90 min., detection 254 nm). Fractions containing the desired product were combined, evaporated in vacuum to provide target product (472 mg (60%) of 5-Amino-2-(6-iodo-benzo[1,3]dioxol-5-ylsulfanyl)-1-methyl-1H-imidazole-4-carboxamide as white off solid.

### Example 71

### Preparation of 2-(6-iodo-benzol[1,3]dioxol-5-ylsulfanyl)-1-methyl-5-phenylacetylamino-1H-imidazole-4-carboxamide

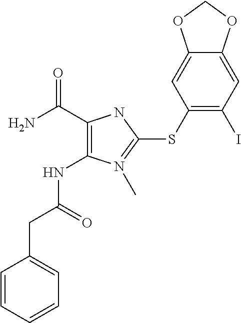

A solution of compound 5-amino-2-(6-iodo-benzo[1,3]dioxol-5-ylsulfanyl)-1-methyl-1H-imidazole-4-carboxamide (25 mg, 0.06 mmol), DIEA (21 μl, 0.12 mmol) and phenyl-acetyl chloride (98%, Aldrich, 10 μl, 0.075 mmol) in CHCl₃ (500 μl) was maintained at ambient temperature for 24 hours. Solvent was evaporated; residue was dissolved in 500 μl of DMSO and submitted for HPLC-purification. Fractions containing the desired product were combined and dried in vacuum (Savant) to provide target product (7 mg (18%) of) 2-(6-Iodo-benzo[1,3]dioxol-5-ylsulfanyl)-1-methyl-5-phenylacetylamino-1H-imidazole-4-carboxamide as trifluoroacetic salt. EI MS: m/z=537 (M⁺+1).

### Example 72

### Preparation 2-(5-iodobenzo[d][1,3]dioxol-6-ylthio)-1-methyl-5-(phenethylamino)-1H-imidazole-4-carboxamide

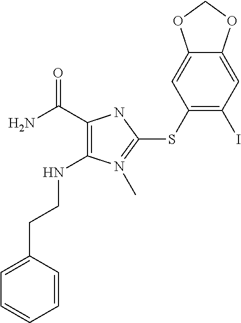

A mixture of compound 5-amino-2-(6-iodo-benzo[1,3]dioxol-5-ylsulfanyl)-1-methyl-1H-imidazole-4-carboxamide (60 mg, 0.143 mmol), phenyl-acetaldehyde (75 mg, 0.572 mmol) and silica supported cyanoborohydride (240 mg, 1.0 mmol/g) in 600 μl of 95/5 ethanol/acetic acid was irradiated under microwave conditions 25 minutes at 120° C. (Biotage, Initiator™ Sixty). The vial was centrifuged to condense silica and the solution transferred by pipette to 20 mL vial. The silica residue was rinsed with an additional 500 μl ethanol, centrifuged and combined with first solutions. Reaction mixture was concentrated in vacuum; residue was dissolved in 500 μl of DMSO and submitted for HPLC-purification. Fractions containing the desired product were combined and dried in vacuum (Savant) to provide target product (2.4 mg (2.6%) of 2-(5-iodobenzo[d][1,3]dioxol-6-ylthio)-1-methyl-5-(phenethylamino)-1H-imidazole-4-carboxamide as trifluoroacetic salt. EI MS: m/z=523 (M⁺+1).

### Example 73

### Preparation of 5-amino-2-[(6-bromo-1,3-benzodioxol-5-yl)thio]-1-methyl-1H-imidazole-4-carboxamide

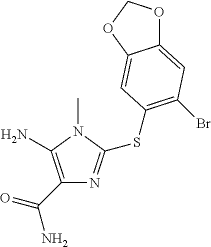

A suspension of 5-amino-2-mercapto-1-methyl-1H-imidazole-4-carboxamide (ChemBridge, 0.393 g, 2.28 mmol), 1,2-dibromo-4,5-(methylenedioxy)benzene (1.28 g, 4.57 mmol), t-BuOK (0.258 g, 2.30 mmol), (oxydi-2,1-phenylene)-bis(diphenylphosphine) (0.122 g, 0.228 mmol) and Pd₂(dba)₃ (0.209 g, 0.228 mmol) in DMF (15 mL) was warmed up to 100° C., and allowed to react for 5 h. Solvent was concentrated off and the crude residue was purified by flash chromatography on SiO₂ (10% MeOH/CH₂Cl₂/NH₃) to furnish 5-amino-2-[(6-bromo-1,3-benzodioxol-5-yl)thio]-1-methyl-1H-imidazole-4-carboxamide (0.519 g, off-white solid, yield: 61%). ¹H NMR (DMSO-d₆, 250 MHz) δ ppm: 7.28 (s, 1H), 7.08 (bs, 1H), 6.77 (bs, 1H), 6.14 (bs, 2H), 6.13 (s, 1H), 6.04 (s, 2H), 3.30 (s, 3H). EI MS: m/z=371 (M+1).

### Example 74

### Preparation of 5-Bromo-6-isopropyl-benzol[1,3]dioxole

n-BuLi (2.5 M in hexanes, 7.3 mL, 18.27 mmol) was added to a −78° C. cooled solution of methyltriphenylphosphonium bromide (6.52 g, 18.27 mmol) in THF (100 mL). After 15 min, a solution of 3′,4′-(methylenedioxy)acetophenone (2 g, 12.18 mmol) in THF (50 mL) was added and mixture was stirred at low temperature for 1 h. The reaction mixture was poured into H₂O (100 mL) and extracted with EtOAc (200 mL). The organic layer was dried over Na₂SO₄ (anhydrous), filtered and concentrated. The crude residue was purified by flash chromatography on SiO₂ (5% EtOAc/hexanes) to furnish 5-isopropenyl-1,3-benzodioxole (0.518 g, white solid, yield: 65%). ¹H NMR (CDCl₃, 250 MHz) δ ppm: 6.97 (m, 2H), 6.77 (m, 1H), 5.96 (s, 2H), 5.27 (s, 1H), 5.00 (s, 1H), 2.11 (s, 3H).

A suspension of 5-isopropenyl-1,3-benzodioxole (1.25 g, 7.70 mmol) and Pd/C (0.82 g, 10% palladium on activated carbon, 0.77 mmol) in EtOAc (20 mL) was stirred under H₂ atmosphere (balloon) for 1.5 h. It was filtered through Celite, washed with AcOEt, and concentrated, to furnish 5-isopropyl-1,3-benzodioxole (1.21 g, pale yellow oil, yield: 96%). The crude residue was submitted to next step without purification. ¹H NMR (CDCl₃, 250 MHz) δ ppm: 6.75-6.60 (m, 3H), 5.89 (s, 2H), 2.81 (m, 1H), 1.20 (s, 3H), 1.17 (s, 3H).

NBS (1.43 g, 8.03 mmol) was added to a solution of 5-isopropyl-1,3-benzodioxole (1.2 g, 7.3 mmol) in CH₃CN (25 mL). The reaction mixture was stirred at room temperature for 1.5 h, poured into H₂O (25 mL) and extracted with EtOAc (25 mL). The organic layer was dried over Na₂SO₄ (anhydrous), filtered and concentrated. The crude residue was purified by flash chromatography on SiO₂ (0→5% EtOAc/hexanes) to furnish 5-bromo-6-isopropyl-benzo[1,3]dioxole (1.5 g, yellow oil, yield: 99%). ¹H NMR (CDCl₃, 250 MHz) δ ppm: 6.97 (s, 1H), 6.75 (s, 1H), 5.92 (s, 2H), 3.28 (m, 1H), 1.18 (s, 3H), 1.17 (s, 3H).

### Example 75

### Preparation of 5-amino-2-[(6-isopropyl-1,3-benzodioxol-5-yl)thio]-1-methyl-1H-imidazole-4-carboxamide

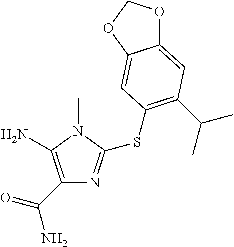

The compound was synthesized from 5-amino-2-mercapto-1-methyl-1H-imidazole-4-carboxamide and 5-bromo-6-isopropyl-benzo[1,3]dioxole following the experimental procedure detailed in Example 73. It was purified by flash chromatography on SiO₂ (5% MeOH/CH₂Cl₂) to yield a brown solid that was precipitated from Et₂O (yield: 10%). ¹H NMR (DMSO-d₆, 250 MHz) δ ppm: 7.01 (sa, 1H), 6.95 (s, 1H), 6.76 (bs, 1H), 6.33 (s, 1H), 6.06 (s, 2H), 5.95 (s, 2H), 3.47 (m, 1H), 3.30 (s, 3H), 1.18 (s, 3H), 1.15 (s, 3H). EI MS: m/z=335 (M+1).

### Example 76

### Preparation of 5-bromo-6-vinyl-1,3-benzodioxole

n-BuLi (2.5 M in hexanes, 10.47 mL, 26.19 mmol) was added to a −78° C. cooled solution of methyltriphenylphosphonium bromide (9.35 g, 26.19 mmol) in THF (300 mL). The reaction mixture was stirred at low temperature for 10 min and a solution of 6-bromo-1,3-benzodioxole-5-carboxaldehyde (4 g, 17.46 mmol) in THF (50 mL) was added. After 1 h, the reaction was poured into H₂O (250 mL) and extracted with EtOAc (300 mL). The organic layer was dried over Na₂SO₄ (anhydrous), filtered and concentrated. The crude residue was purified by flash chromatography on SiO₂ (10% EtOAc/hexanes) to furnish 5-bromo-6-vinyl-1,3-benzodioxole (2.96 g, yellow oil, yield: 75%). ¹H NMR (CDCl₃, 250 MHz) δ ppm: 7.03 (s, 1H), 6.99 (s, 1H), 6.97 (m, 1H), 5.97 (s, 2H), 5.55 (d, J=17.3 Hz, 1H), 5.25 (d, J=10.7 Hz, 1H).

### Example 77

### Preparation of 5-amino-1-methyl-2-[(6-vinyl-1,3-benzodioxol-5-yl)thio]-1H-imidazole-4-carboxamide

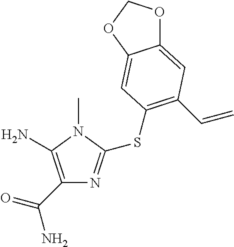

The compound was synthesized from of 5-amino-2-mercapto-1-methyl-1H-imidazole-4-carboxamide and 5-bromo-6-vinyl-1,3-benzodioxole following the experimental procedure detailed in Example 73. It was purified by flash chromatography on SiO₂ (5% MeOH/CH₂Cl₂) to yield a pale orange solid that was slurred with Et₂O (5 mL) to furnish an off-white solid (yield: 41%). ¹H NMR (DMSO-d₆, 250 MHz) δ ppm: 7.24 (s, 1H), 7.18 (dd, J₁=10.5 Hz, J₂=6.3 Hz 1H), 7.05 (bs, 1H), 6.77 (bs, 1H), 6.42 (s, 1H), 6.08 (bs, 2H), 6.02 (s, 2H), 5.75 (d, J=17.0 Hz, 1H), 5.31 (d, J=11.0 Hz, 1H). EI MS: m/z=319 (M+1).

### Example 78

### Preparation of 5-Bromo-6-ethoxy-1,3-benzodioxole

Iodoethane (0.692 mL, 8.68 mmol) was added to a suspension sesamol (1 g, 7.24 mmol) and K₂CO₃ (1.5 g, 10.86 mmol) in DMF (25 mL). The reaction mixture was stirred at room temperature for 1.5 h, poured into H₂O (50 mL) and extracted with EtOAc (2×50 mL). The organic layer was dried over Na₂SO₄ (anhydrous), filtered and concentrated. The crude residue was purified by flash chromatography on SiO₂ (5% EtOAc/hexanes) to furnish 5-ethoxy-1,3-benzodioxole (0.75 g, off-white solid, yield: 62%). ¹H NMR (CDCl₃, 250 MHz) δ ppm: 6.70 (d, J=8.5 Hz, 1H), 6.49 (d, J=2.5 Hz, 1H), 6.32 (dd, J₁=8.5 Hz, J₂=2.7 Hz, 1H), 5.91 (s, 2H), 3.95 (c, J₁=14 Hz, J₂=6.9 Hz, 2H), 1.38 (t, J=7.1 Hz, 3H).

NBS (0.883 g, 4.96 mmol) was added to a solution of 5-ethoxy-1,3-benzodioxole (0.75 g, 4.51 mmol) in CH₃CN (30 mL). The reaction mixture was stirred at room temperature for 30 min, poured into H₂O (30 mL) and extracted with EtOAc (2×50 mL). The organic layer was dried over Na₂SO₄ (anhydrous), filtered and concentrated. The crude residue was purified by flash chromatography on SiO₂ (5% EtOAc/hexanes) to furnish 5-bromo-6-ethoxy-1,3-benzodioxole (0.93 g, white solid, yield: 85%). ¹H NMR (CDCl₃, 250 MHz) δ ppm: 6.98 (s, 1H), 6.55 (s, 1H), 5.93 (s, 2H), 4.00 (c, J₁=14 Hz, J₂=6.9 Hz, 2H), 1.42 (t, J=7.1 Hz, 3H).

### Example 79

### Preparation of 5-amino-2-[(6-ethoxy-1,3-benzodioxol-5-yl)thio]-1-methyl-1H-imidazole-4-carboxamide

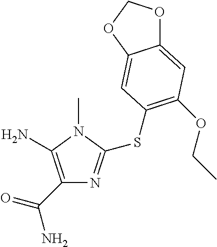

The compound was synthesized from of 5-amino-2-mercapto-1-methyl-1H-imidazole-4-carboxamide and 5-bromo-6-ethoxy-1,3-benzodioxole following the experimental procedure detailed in Example 73. It was purified by flash chromatography on SiO₂ (5% MeOH/CH₂Cl₂) to yield a purple solid that was slurred with Et₂O (5 mL) to furnish a pale purple solid (yield: 9%). ¹H NMR (DMSO-d₆, 250 MHz) δ ppm: 7.01 (bs, 1H), 6.86 (s, 1H), 6.74 (bs, 1H), 6.15 (s, 1H), 6.05 (bs, 2H), 5.94 (s, 2H), 4.03 (c, J₁=13.7 Hz, J₂=6.8 Hz, 2H), 3.34 (s, 3H), 1.29 (t, J=6.8 Hz, 3H). EI MS: m/z=337 (M+1).

### Example 80

### Preparation of 5-bromo-6-(methylthio)-1,3-benzodioxole

n-BuLi (2.5 M in hexanes, 6.5 mL, 16.41 mmol) was added to a −78° C. cooled solution of 1-bromo-3,4-(methylenedioxy)benzene (3 g, 14.92 mmol) in THF (30 mL). The reaction mixture was stirred at low temperature for 30 min and methyl disulfide (2.64 mL, 29.84 mmol) was added. After 30 min, the mixture was poured into H₂O (50 mL) and extracted with EtOAc (30 mL). The organic layer was dried over Na₂SO₄ (anhydrous), filtered and concentrated. The crude residue was purified by flash chromatography on SiO₂ (2% EtOAc/hexanes) to furnish 5-(methylthio)-1,3-benzodioxole (2.28 g, colourless oil, yield: 91%). ¹H NMR (CDCl₃, 250 MHz) δ ppm: 6.87-6.69 (m, 3H), 5.94 (s, 2H), 2.94 (s, 3H).

NBS (2.65 g, 14.90 mmol) was added to a solution of 5-(methylthio)-1,3-benzodioxole (2.28 g, 13.55 mmol) in CH₃CN (25 mL). The reaction mixture was stirred at room temperature for 30 min, poured into H₂O (50 mL) and extracted with EtOAc (2×50 mL). The organic layer was dried over Na₂SO₄ (anhydrous), filtered and concentrated. The crude residue was purified by flash chromatography on SiO₂ (0 to 2% EtOAc/hexanes) to furnish 5-bromo-6-(methylthio)-1,3-benzodioxole (1.75 g, yellow oil, yield: 52%). ¹H NMR (CDCl₃, 250 MHz) δ ppm: 7.02 (s, 1H), 6.75 (s, 1H), 5.97 (s, 2H), 2.43 (s, 3H).

### Example 81

### Preparation of 5-amino-1-methyl-2-{[6-(methylthio)-1,3-benzodioxol-5-yl]thio}-1H-imidazole-4-carboxamide

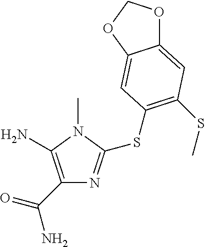

The compound was synthesized from of 5-amino-2-mercapto-1-methyl-1H-imidazole-4-carboxamide and 5-bromo-6-(methylthio)-1,3-benzodioxole following the experimental procedure detailed in Example 73. It was purified by flash chromatography on SiO₂ (5% MeOH/CH₂Cl₂) and SiO₂ (10 to 20% ⁱPrOH/CHCl₃) to yield a brown solid that was slurred with Et₂O (5 mL) to furnish an off-white solid (yield: 51%). ¹H NMR (DMSO-d₆, 250 MHz) δ ppm: 7.07 (bs, 1H), 7.02 (s, 1H), 6.77 (bs, 1H), 6.19 (s, 1H), 6.09 (bs, 2H), 6.00 (s, 2H), 3.32 (s, 3H), 2.46 (s, 3H). EI MS: m/z=339 (M+1).

### Example 82

### Preparation of 5-bromo-6-(methylsulfonyl)-1,3-benzodioxole

Potassium peroxomonosulfate (Oxone®, 3.73 g, 6.07 mmol) was added to a solution of 5-bromo-6-(methylthio)-1,3-benzodioxole (0.5 g, 2.02 mmol) in THF/MeOH/H₂O (5:5:5 mL). The reaction mixture was stirred at room temperature for 2.5 h. Extra Oxone® (2.48 g, 4.04 mmol) was added and the reaction mixture was stirred at room temperature for 1 h, poured into H₂O (20 mL) and extracted with EtOAc (2×30 mL). The organic layer was dried over Na₂SO₄ (anhydrous), filtered and concentrated. The crude residue was purified by flash chromatography on SiO₂ (40% EtOAc/hexanes) to furnish 5-bromo-6-(methylsulfonyl)-1,3-benzodioxole (0.437 g, white solid, yield: 78%). ¹H NMR (CDCl₃, 250 MHz) δ ppm: 7.62 (s, 1H), 7.17 (s, 1H), 6.11 (s, 2H), 3.24 (s, 3H).

### Example 83

### Preparation of 5-amino-1-methyl-2-{[6-(methylsulfonyl)-1,3-benzodioxol-5-yl]thio}-1H-imidazole-4-carboxamide

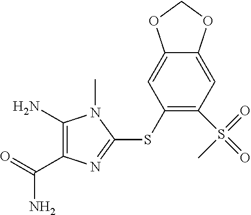

The compound was synthesized from of 5-amino-2-mercapto-1-methyl-1H-imidazole-4-carboxamide and 5-bromo-6-(methylsulfonyl)-1,3-benzodioxole following the experimental procedure detailed in Example 73. It was purified by flash chromatography on SiO₂ (5% MeOH/CH₂Cl₂) to yield a pink solid that was slurred with Et₂O (5 mL) to furnish an off-white solid (yield: 30%). ¹H NMR (DMSO-d₆, 250 MHz) δ ppm: 7.38 (s, 1H), 7.13 (bs, 1H), 6.81 (bs, 1H), 6.21 (bs, 1H), 6.16 (bs, 2H), 5.76 (s, 2H), 3.39 (s, 3H), 3.33 (s, 3H).

### Example 84

### Preparation of 5,6-diiodo-1,3-benzodioxole

I₂ (19.5 g, 76.96 mmol) was added in portions to a suspension of 1,3-benzodioxole (1.9 g, 15.39 mmol) and AgCO₂CF₃ (17 g, 76.96 mmol) in CHCl₃ (100 mL). The reaction mixture was refluxed for 24 h, allowed to reach room temperature and filtered through Celite. The organic layer was washed with Na₂S₂O₃ (saturated aqueous solution, 2×200 mL), dried over Na₂SO₄ (anhydrous), filtered and concentrated. The crude residue was purified by flash chromatography on SiO₂ (3% Et₂O/Hexanes) to furnish 5,6-diiodo-1,3-benzodioxole (1.94 g, white solid, yield: 34%). ¹H NMR (CDCl₃, 250 MHz) δ ppm: 7.31 (s, 2H), 5.98 (s, 2H).

### Example 85

### Preparation of 5-amino-2-[(6-iodo-1,3-benzodioxol-5-yl)thio]-1-methyl-1H-imidazole-4-carboxamide

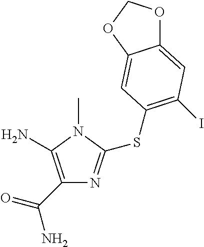

The compound was synthesized from of 5-amino-2-mercapto-1-methyl-1H-imidazole-4-carboxamide and 5,6-diiodo-1,3-benzodioxole following the experimental procedure detailed in Example 73. It was purified by flash chromatography on SiO₂ (5% MeOH/CH₂Cl₂) and SiO₂ (2 to 5% MeOH/CH₂Cl₂) to yield colourless oil (yield: 10%). EI MS: m/z=419 (M+1).

### Example 86

### Preparation of 5-amino-2-[(6-bromo-1,3-benzodioxol-5-yl)thio]-1-(3-morpholin-4-ylpropyl)-1H-imidazole-4-carboxamide

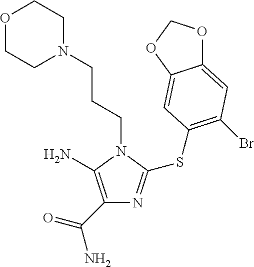

The compound was synthesized from of 5-amino-2-mercapto-1-(3-morpholin-4-ylpropyl)-1H-imidazole-4-carboxamide previously prepared from 3-morpholinopropyl isothiocyanate following the method described of cf. Cook H. A. et al., “Studies in the Azole Series. Part XVII. The preparation and cyclisation reactions of aminocyanoacetamide”, J. Chem. Soc., 1949, p. 1440) and 1,2-dibromo-4,5-(methylenedioxy)benzene following the experimental procedure detailed in Example 73. It was purified by flash chromatography on SiO₂ (5 to 10% MeOH/CH₂Cl₂) to yield an orange oil (yield: 31%). ¹H NMR (DMSO-d₆, 250 MHz) δ ppm: 7.94 (bs, 1H), 7.26 (s, 1H), 6.95 (bs, 1H), 6.73 (bs, 2H), 6.66 (s, 1H), 6.22 (s, 2H), 4.13 (m, 2H), 4.00 (m, 4H), 3.75 (bs, 2H), 2.71 (m, 4H), 2.13 (m, 2H).

### Example 87

### Preparation of 5-amino-2-[(6-iodo-1,3-benzodioxol-5-yl)thio]-1-(3-morpholin-4-ylpropyl)-1H-imidazole-4-carboxamide

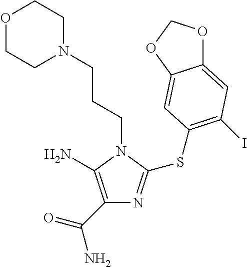

The compound was synthesized from of 5-amino-2-mercapto-1-(3-morpholin-4-ylpropyl)-1H-imidazole-4-carboxamide previously prepared from 3-morpholinopropyl isothiocyanate following the method described of cf. Cook H. A. et al., supra) and 5,6-diiodo-1,3-benzodioxole following the experimental procedure detailed in Example 73. It was purified by flash chromatography on SiO₂ (5% MeOH/CH₂Cl₂) to yield a yellow solid that was slurred with Et₂O to give an off-white solid (yield: 6%). ¹H NMR (DMSO-d₆, 250 MHz) δ ppm: 7.41 (s, 1H), 7.13 (bs, 1H), 6.80 (bs, 1H), 6.28 (bs, 2H), 6.18 (s, 1H), 6.01 (s, 2H), 3.83 (m, 2H), 3.54 (m, 4H), 2.22 (m, 6H), 1.63 (m, 2H).

### Example 88

### Preparation of 5-Amino-2-[(6-iodo-1,3-benzodioxol-5-yl)thio]-1-[3-(diethylamino)propyl]-1H-imidazole-4-carboxamide (CRY-267)

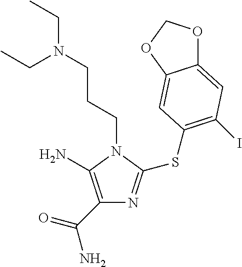

The compound was synthesized from of 5-amino-1-[3-(diethylamino)propyl]-2-mercapto-1H-imidazole-4-carboxamide previously prepared from 3-(diethylamino)propyl isothiocyanate following the method described of cf. Cook H. A. et al., supra) and 5,6-diiodo-1,3-benzodioxole following the experimental procedure detailed in Example 73. It was purified by flash chromatography on SiO₂ (5% MeOH/CH₂Cl₂/NH₃) and then by HPLC to yield an off-white solid that was slurred with Et₂O to give a white solid (yield: 8%). ¹H NMR (DMSO-d₆, 250 MHz) δ ppm: 7.39 (s, 1H), 7.13 (bs, 1H), 6.80 (bs, 1H), 6.27 (bs, 2H), 6.19 (s, 1H), 6.02 (s, 2H), 3.79 (m, 2H), 2.42-2.16 (m, 6H), 1.55 (m, 2H), 0.87 (t, J=7.1 Hz, 6H). EI MS: m/z=518 (M+1).

### Example 89

### Preparation of 5-amino-2-[(6-bromo-1,3-benzodioxol-5-yl)thio]-1-[3-(diethylamino)propyl]-1H-imidazole-4-carboxamide

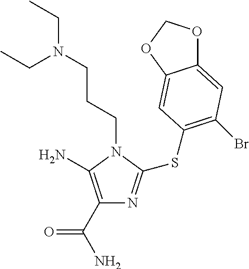

The compound was synthesized from of 5-amino-1-[3-(diethylamino)propyl]-2-mercapto-1H-imidazole-4-carboxamide previously prepared from 3-(diethylamino)propyl isothiocyanate following the method described of cf. Cook H. A. et al., supra) and 1,2-dibromo-4,5-(methylenedioxy)benzene following the experimental procedure detailed in Example 73. It was purified by flash chromatography on SiO₂ (10 to 30% MeOH/CH₂Cl₂) to yield an off-white solid that was slurred with Et₂O to give a white solid (yield: 23%). ¹H NMR (DMSO-d₆, 250 MHz) δ ppm: 7.29 (s, 1H), 7.14 (bs, 1H), 6.81 (bs, 1H), 6.29 (bs, 2H), 6.19 (s, 1H), 6.04 (s, 2H), 3.82 (m, 2H), 2.54-2.11 (m, 6H), 1.60 (m, 2H), 0.89 (m, 6H).

### Example 90

### Preparation of 2-[(6-bromo-1,3-benzodioxol-5-yl)thio]-1-methyl-5-[(methylsulfonyl)amino]-1H-imidazole-4-carboxamide

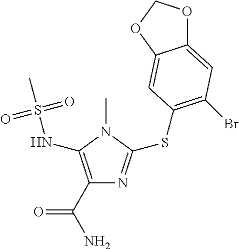

Methanesulfonyl chloride (0.27 mL, 0.349 mmol) was added to a suspension 5-amino-2-[(6-bromo-1,3-benzodioxol-5-yl)thio]-1-methyl-1H-imidazole-4-carboxamide (0.3 g, 0.808 mmol) and Et₃N (0.338 mL, 2.42 mmol) in CH₂Cl₂ (20 mL). The reaction mixture was stirred at room temperature for 4 h, poured into H₂O (25 mL) and extracted with CH₂Cl₂ (30 mL). The organic layer was dried over Na₂SO₄ (anhydrous), filtered and concentrated. The crude residue was purified by flash chromatography on SiO₂ (5% MeOH/CH₂Cl₂) to yield a orange solid that was slurred with CH₂Cl₂ (5 mL), to furnish 5-[bis(methylsulfonyl)amino]-2-[(6-bromo-1,3-benzodioxol-5-yl)thio]-1-methyl-1H-imidazole-4-carboxamide (0.22 g, off-white solid, yield: 52%). ¹H NMR (DMSO-d₆, 250 MHz) δ ppm: 7.74 (bs, 1H), 7.46 (bs, 1H), 7.34 (s, 1H), 6.27 (s, 1H), 6.07 (s, 2H), 3.64 (s, 6H), 3.58 (s, 3H).

LiOH (1 mL, 2 M solution in H₂O, 1.9 mmol) was added to a solution of 5-[bis(methylsulfonyl)amino]-2-[(6-bromo-1,3-benzodioxol-5-yl)thio]-1-methyl-1H-imidazole-4-carboxamide (0.2 g, 0.37 mmol) in THF/MeOH (5:5 mL). The reaction mixture was stirred at room temperature. for 1.5 h and poured into H₂O (5 mL). The mixture was taken up to pH 4 with HCl (10% aqueous solution), and extracted with CH₂Cl₂ (10 mL). The organic layer was dried over Na₂SO₄ (anhydrous), filtered and concentrated. The crude residue was purified by flash chromatography on SiO₂ (5% MeOH/CH₂Cl₂) to yield a off-white solid that was slurred with Et₂O (5 mL), to furnish CRY-265 (0.108 g, off-white solid, yield: 65%). ¹H NMR (DMSO-d₆, 250 MHz) δ ppm: 10.02 (bs, 1H), 7.63 (bs, 1H), 7.32 (s, 2H), 6.28 (s, 1H), 6.07 (s, 2H), 3.50 (s, 3H), 3.11 (s, 3H). EI MS: m/z=449 (M+1).

### Example 91

### Preparation of 5-(acetylamino)-2-[(6-bromo-1,3-benzodioxol-5-yl)thio]-1-methyl-1H-imidazole-4-carboxamide

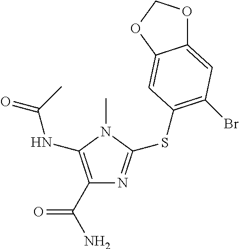

Ac₂O (0.2 mL, 0.2.11 mmol) was added to a solution of 5-amino-2-[(6-bromo-1,3-benzodioxol-5-yl)thio]-1-methyl-1H-imidazole-4-carboxamide (0.25 g, 0.673 mmol) in DCE (200 mL). The reaction mixture was warmed up to reflux for 24 h. Solvent was concentrated off and the crude residue was purified by flash chromatography on SiO₂ (2% MeOH/CH₂Cl₂) to furnish 2-[(6-bromo-1,3-benzodioxol-5-yl)thio]-5-(diacetylamino)-1-methyl-1H-imidazole-4-carboxamide (0.22 g, off-white solid, yield: 66%). ¹H NMR (CDCl₃, 250 MHz) δ ppm: 7.00 (s, 2H), 6.48 (s, 1H), 5.95 (s, 2H), 5.91 (bs, 1H), 3.40 (s, 3H), 2.33 (s, 6H). EI MS: m/z=455 (M+1).

LiOH (1.2 mL, 2 M solution in H₂O, 2.4 mmol) was added to a solution of 2-[(6-bromo-1,3-benzodioxol-5-yl)thio]-5-(diacetylamino)-1-methyl-1H-imidazole-4-carboxamide (0.22 g, 0.483 mmol) in THF/MeOH (5:5 mL). The reaction mixture was stirred at room temperature. for 2 h and poured into H₂O (10 mL). It was taken up to pH 3 with HCl (10% aqueous solution), and extracted with CH₂Cl₂ (2×10 mL). The organic layer was dried over Na₂SO₄ (anhydrous), filtered and concentrated. The crude residue was purified by flash chromatography on SiO₂ (5% MeOH/CH₂Cl₂) to yield an off-white solid that was precipitated from Et₂O (5 mL), to furnish 5-(acetylamino)-2-[(6-bromo-1,3-benzodioxol-5-yl)thio]-1-methyl-1H-imidazole-4-carboxamide (0.140 g, off-white solid, yield: 70%). ¹H NMR (DMSO-d₆, 250 MHz) δ ppm: 10.13 (bs, 1H), 7.49 (bs, 1H), 7.31 (s, 1H), 7.18 (bs, 1H), 6.19 (s, 1H), 6.05 (s, 2H), 3.33 (s, 3H), 2.07 (s, 3H). EI MS: m/z=413 (M+1).

### Example 92

### Preparation of 4-amino-1-(6-bromo-benzol[1,3]dioxol-5-ylmethyl)-5-methyl-1H-pyrazole-3-carboxamide

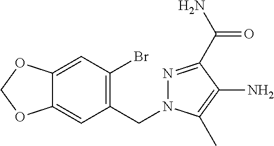

Hydrazine monohydrate (5.4 mL, 110.68 mmol) was added to a 0° C. cooled solution of ethyl 2,4-dioxopentanoate (11.67 g, 73.79 mmol) in EtOH/AcOH (100/1 mL). The reaction mixture was stirred at room temperature. for 15 h, poured into H₂O (50 mL) and NaHCO₃ (saturated aqueous solution, 5 mL) and extracted with EtOAc (3×50 mL). The organic layer was dried over Na₂SO₄ (anhydrous), filtered and concentrated, to furnish ethyl 5-methyl-1H-pyrazole-3-carboxylate, which was submitted to next step without further purification (8.41 g, white solid, yield: 74%). ¹H NMR (CDCl₃, 250 MHz): 6 ppm 6.55 (s, 1H), 4.34 (c, J=7.13 Hz, 2H), 2.35 (s, 3H), 1.33 (t, J=7.13 Hz, 3H). EI MS: m/z=155 (M+1).

5-Bromo-6-bromomethyl-1,3-benzodioxole (2 g, 6.78 mmol) was added to a solution of ethyl 5-methyl-4-nitro-1H-pyrazole-3-carboxylate (1.0 g, 5.02 mmol), Cs₂CO₃ (2.45 g, 7.53 mmol) in CH₃CN (50 mL). The reaction mixture was warmed up to reflux for 2 h, poured into H₂O (50 mL) and NaHCO₃ (saturated aqueous solution, 5 mL) and extracted with EtOAc (50 mL). The organic layer was dried over Na₂SO₄ (anhydrous), filtered and concentrated. The crude residue was purified by flash chromatography on SiO₂ (0 to 30% EtOAc/hexanes), to afford ethyl 1-[(6-bromo-1,3-benzodioxol-5-yl)methyl]-5-methyl-4-nitro-1H-pyrazole-3-carboxylate (0.855 g, yellow solid, yield: 42%). ¹H NMR (CDCl₃, 250 MHz): 6 ppm 7.03 (s, 1H), 6.41 (s, 1H), 5.98 (s, 2H), 5.36 (s, 2H), 4.45 (c, J=7.14 Hz, 2H), 2.54 (s, 3H), 1.40 (t, J=7.13 Hz, 3H). EI MS: m/z=414 (M+1).

NaOH (0.8 mL, 10 M solution in H₂O, 8 mmol) was added to a solution of ethyl 1-[(6-bromo-1,3-benzodioxol-5-yl)methyl]-5-methyl-4-nitro-1H-pyrazole-3-carboxylate (0.978 g, 2.37 mmol) in MeOH (10 mL). The reaction mixture was warmed up to reflux for 30 min. It was allowed to reach room temperature. and poured into H₂O (10 mL). The mixture was taken up to pH 5 with HCl (10% aqueous solution), and extracted with CH₂Cl₂ (5×15 mL). The organic layer was dried over Na₂SO₄ (anhydrous), filtered and concentrated to furnish 1-[(6-bromo-1,3-benzodioxol-5-yl)methyl]-5-methyl-4-nitro-1H-pyrazole-3-carboxylic acid, which was submitted to next step without further purification (0.5 g, off-white solid, yield: 55%). ¹H NMR (CDCl₃, 250 MHz): 6 ppm 7.04 (s, 1H), 6.46 (s, 1H), 5.99 (s, 2H), 5.44 (s, 2H), 2.59 (s, 3H).

A solution of 1-[(6-bromo-1,3-benzodioxol-5-yl)methyl]-5-methyl-4-nitro-1H-pyrazole-3-carboxylic acid (0.5 g, 1.30 mmol) in SOCl₂ (5 mL) was warmed up to reflux, and allowed to react for 2 h. Solvent was concentrated off, THF (20 mL) was added and the mixture was concentrated again. The crude residue was taken up in THF (5 mL), cooled down to −10° C. and NH₃ (2M in EtOH, 2 mL) was added. The reaction mixture was stirred at room temperature for 1 h and it was concentrated to furnish 1-[(6-bromo-1,3-benzodioxol-5-yl)methyl]-5-methyl-4-nitro-1H-pyrazole-3-carboxamide, which was submitted to next step without further purification (off-white solid). ¹H NMR (DMSO-d₆, 250 MHz): δ ppm 8.01 (s, 1H), 7.71 (s, 1H), 7.30 (s, 1H), 6.64 (s, 1H), 6.06 (s, 2H), 5.36 (s, 2H), 2.61 (s, 3H). EI MS: m/z=385 (M+1).

SnCl₂.H₂O (0.879 g, 3.9 mmol) was added to a suspension of 1-[(6-bromo-1,3-benzodioxol-5-yl)methyl]-5-methyl-4-nitro-1H-pyrazole-3-carboxamide (0.498 g, 1.30 mmol) in EtOH (10 mL). The reaction mixture was warmed up to reflux for 2 h. Solvent was concentrated and off the crude residue was poured into H₂O (15 mL). It was taken up to pH 9 with NaOH (10% aqueous solution), and extracted with CH₂Cl₂ (2×20 mL). The organic layer was dried over Na₂SO₄ (anhydrous), filtered and concentrated. The crude residue was purified by flash chromatography on SiO₂ (5% MeOH/CH₂Cl₂) to furnish 4-amino-1-(6-bromo-benzo[1,3]dioxol-5-ylmethyl)-5-methyl-1H-pyrazole-3-carboxamide (0.261 g, off-white solid, yield: 57%). ¹H NMR (CD₃OD, 250 MHz): δ ppm 7.09 (s, 1H), 6.12 (s, 1H), 5.96 (s, 2H), 5.26 (s, 2H), 2.12 (s, 3H). EI MS: m/z=355 (M+1).

### Example 93

### Preparation of 4-(acetylamino)-1-[(6-bromo-1,3-benzodioxol-5-yl)methyl]-5-methyl-1H-pyrazole-3-carboxamide

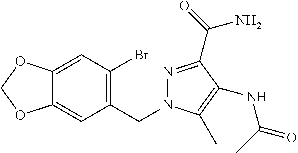

Ac₂O (0.022 mL, 0.237 mmol) was added to a solution of 4-amino-1-(6-bromo-benzo[1,3]dioxol-5-ylmethyl)-5-methyl-1H-pyrazole-3-carboxamide (0.07 g, 0.198 mmol) in CH₂Cl₂ (5 mL). The reaction mixture was stirred at room temperature for 1 h, poured into H₂O (5 mL) and extracted with CH₂Cl₂ (2×5 mL). The organic layer was dried over Na₂SO₄ (anhydrous), filtered and concentrated. The crude residue was purified by flash chromatography on SiO₂ (5% MeOH/CH₂Cl₂) to yield a viscous oil that was precipitated from Et₂O (2 mL), to furnish 4-(acetylamino)-1-[(6-bromo-1,3-benzodioxol-5-yl)methyl]-5-methyl-1H-pyrazole-3-carboxamide (0.051 g, off-white solid, yield: 65%). ¹H NMR (CD₃OD, 250 MHz): 6 ppm 7.10 (s, 1H), 6.31 (s, 1H), 5.97 (s, 2H), 5.34 (s, 2H), 2.14 (s, 3H), 2.13 (s, 3H). EI MS: m/z=396 (M+1).

### Example 94

### Preparation of 1-[(6-bromo-1,3-benzodioxol-5-yl)methyl]-5-methyl-4-(propionylamino)-1H-pyrazole-3-carboxamide

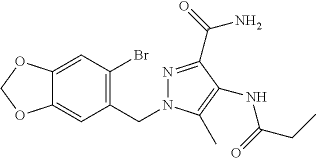

Propionyl chloride (0.02 mL, 0.237 mmol) was added to a solution of 4-amino-1-(6-bromo-benzo[1,3]dioxol-5-ylmethyl)-5-methyl-1H-pyrazole-3-carboxamide (0.07 g, 0.198 mmol) and Et₃N (0.04 mL, 0.297 mmol) in CH₂Cl₂ (5 mL). The reaction mixture was stirred at room temperature. for 1 h, poured into H₂O (5 mL) and extracted with CH₂Cl₂ (2×5 mL). The organic layer was dried over Na₂SO₄ (anhydrous), filtered and concentrated. The crude residue was purified by flash chromatography on SiO₂ (5% MeOH/CH₂Cl₂) to yield a viscous oil that was precipitated from Et₂O (2 mL), to furnish 1-[(6-bromo-1,3-benzodioxol-5-yl)methyl]-5-methyl-4-(propionylamino)-1H-pyrazole-3-carboxamide (0.039 g, off-white solid, yield: 48%). ¹H NMR (CD₃OD, 250 MHz): 6 ppm 7.10 (s, 1H), 6.32 (s, 1H), 5.97 (s, 2H), 5.49 (s, 1H), 5.34 (s, 2H), 2.42 (c, J₁=15.3 Hz, J₂=7.5 Hz, 2H), 2.13 (s, 3H), 1.21 (t, J=7.5 Hz, 3H). EI MS: m/z=410 (M+1).

### Example 95

### Preparation of 1-[(6-bromo-1,3-benzodioxol-5-yl)methyl]-5-methyl-4-[(methylsulfonyl)amino]-1H-pyrazole-3-carboxamide

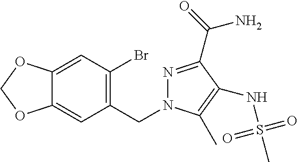

Methanesulfonyl chloride (0.04 mL, 0.474 mmol) was added to a solution of 4-amino-1-(6-bromo-benzo[1,3]dioxol-5-ylmethyl)-5-methyl-1H-pyrazole-3-carboxamide (0.07 g, 0.198 mmol) and Et₃N (0.08 mL, 0.594 mmol) in CH₂Cl₂ (5 mL). The reaction mixture was stirred at room temperature. for 1 h, poured into H₂O (5 mL) and extracted with CH₂Cl₂ (2×5 mL). The organic layer was dried over Na₂SO₄ (anhydrous), filtered and concentrated. The crude residue was purified by flash chromatography on SiO₂ (5% MeOH/CH₂Cl₂) to yield a viscous oil that was precipitated from Et₂O (2 mL), to furnish 1-[(6-bromo-1,3-benzodioxol-5-yl)methyl]-5-methyl-4-[(methylsulfonyl)amino]-1H-pyrazole-3-carboxamide (0.017 g, off-white solid, yield: 36%). ¹H NMR (CD₃OD, 250 MHz): δ ppm 7.11 (s, 1H), 6.30 (s, 1H), 5.98 (s, 2H), 5.49 (s, 1H), 5.35 (s, 2H), 2.92 (s, 3H), 2.26 (s, 3H). EI MS: m/z=432 (M+1).

### Example 96

### Preparation of 5-bromomethyl-6-iodo-benzol[1,3]dioxole

A solution of I₂ (1.68 g, 6.57 mmol) in CHCl₃ (10 mL) was added to a solution of piperonyl alcohol (1 g, 6.57 mmol) and Ag₂CO₃ (1.45 g, 6.57 mmol) in CHCl₃ (70 mL). The reaction mixture was stirred at room temperature for 15 h and filtered through Celite. The organic layer was washed with Na₂S₂O₃ (sat. aqueous solution, 50 mL), dried over Na₂SO₄ (anhydrous), filtered and concentrated. The crude residue was purified by flash chromatography on SiO₂ (0 to 10% MeOH/CH₂Cl₂) to furnish (6-iodo-1,3-benzodioxol-5-yl)methanol (1.04 g, white solid, yield: 57%). ¹H NMR (CDCl₃, 250 MHz): 6 ppm 7.20 (s, 1H), 6.95 (s, 1H), 5.93 (s, 2H), 4.55 (s, 2H).

Phosphorus tribromide (0.417 mL, 4.44 mmol) was added to a −10° C. cooled solution of (6-iodo-1,3-benzodioxol-5-yl)methanol (1.03 g, 3.70 mmol)) in Et₂O (25 mL). The reaction mixture was stirred at low temperature for 1 h, poured into NaHCO₃ (sat. aqueous solution, 30 mL) and extracted with Et₂O (25 mL). The organic layer was dried over Na₂SO₄ (anhydrous), filtered and concentrated to furnish 5-bromomethyl-6-iodo-benzo[1,3]dioxole which was submitted to next step without further purification/characterization (0.75 g, white solid, yield: 60%).

### Example 96

### Preparation of 4-amino-1-[(6-iodo-1,3-benzodioxol-5-yl)methyl]-5-methyl-1H-pyrazole-3-carboxamide

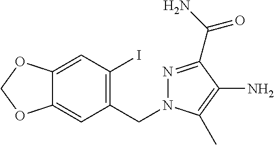

It was prepared following the general method described in Example 92, by using 5-bromomethyl-6-iodo-benzo[1,3]dioxole as alkylating reagent, to furnish 4-amino-1-[(6-iodo-1,3-benzodioxol-5-yl)methyl]-5-methyl-1H-pyrazole-3-carboxamide as an off-white solid after flash chromatography purification (yield: 32%). ¹H NMR (DMSO-d₆, 250 MHz): 6 ppm 7.42 (s, 1H), 7.21 (s, 1H), 7.04 (s, 1H), 6.02 (s, 2H), 5.97 (s, 1H), 5.10 (s, 2H), 4.54 (s, 2H), 2.04 (s, 3H). EI MS: m/z=401 (M+1).

### Example 97

### Preparation of 5-amino-2-[(6-bromo-2,3-dihydro-1-benzofuran-5-yl)thio]-1-methyl-1H-imidazole-4-carboxamide

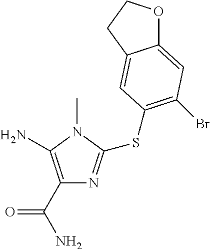

tBuOK (15.46 g, 137.83 mmol) was added to a solution of 1,4-dibromo-2-fluorobenzene (10 g, 39.38 mmol) in a mixture of ethylene glycol (50 mL) and NMP (5 mL). The reaction mixture was warmed up to 100° C. and allowed to react for 3 h. It was poured into H₂O (50 mL) and extracted with EtOAc (2×50 mL). The organic layer was dried over Na₂SO₄ (anhydrous), filtered and concentrated. The crude residue was purified by flash chromatography on SiO₂ (15%→30% EtOAc/hexanes) to furnish 2-(2,5-dibromophenoxy)ethanol (9.22 g, off-white solid, yield: 79%). ¹H NMR (CDCl₃, 250 MHz) δ ppm: 7.39 (d, J=8.2 Hz, 1H), 7.07-6.96 (m, 2H), 4.12 (t, J=4.1 Hz, 2H), 3.99 (t, J=4.1 Hz, 2H).

PBr₃ (6.42 mL, 68.38 mmol) was added to a solution of 2-(2,5-dibromo-phenoxy)ethanol (9.2 g, 31.08 mmol) in toluene (200 mL). The reaction mixture was warmed up to 90° C. and allowed to react for 3 h. It was poured into H₂O (200 mL) and extracted with EtOAc (2×150 mL). The organic layer was dried over Na₂SO₄ (anhydrous), filtered and concentrated. The crude residue was slurred with CH₂Cl₂ and collected by filtration. The crude residue was purified by flash chromatography on SiO₂ (20% EtOAc/hexanes) to furnish 1,4-dibromo-2-(2-bromoethoxy)benzene (3.84 g, white solid, yield: 34%). ¹H NMR (CDCl₃, 250 MHz) δ ppm: 7.4 (d, J=8.7 Hz, 1H), 7.02 (m, 2H), 4.32 (t, J=6.3 Hz, 2H), 3.67 (t, J=6.5 Hz, 2H).

nBuLi (2.5 M in hexane, 5.13 mL, 12.84 mmol) was added to a −78° C. cooled solution of 1,4-dibromo-2-(2-bromoethoxy)benzene (3.84 g, 10.70 mmol) in THF (100 mL). The reaction mixture was stirred at low temperature for 4 h. It was poured into H₂O (100 mL) and extracted with EtOAc (2×100 mL). The organic layer was dried over Na₂SO₄ (anhydrous), filtered and concentrated. The crude residue was purified by flash chromatography on SiO₂ (3% EtOAc/hexanes) to furnish 6-bromo-2,3-dihydro-1-benzofuran (2, g, yellow solid, yield: 94%).

NBS (0.982 g, 5.52 mmol) was added to a solution of 6-bromo-2,3-dihydro-1-benzofuran (1 g, 5.02 mmol) in CH₃CN (30 mL). The reaction mixture was stirred at room temperature for 3 h, poured into H₂O (50 mL) and extracted with EtOAc (2×50 mL). The organic layer was dried over Na₂SO₄ (anhydrous), filtered and concentrated. The crude residue was purified by flash chromatography on SiO₂ (5% EtOAc/hexanes) to furnish 5,6-dibromo-2,3-dihydro-1-benzofuran (1.3 g, yellow oil, yield: 93%). ¹H NMR (CDCl₃, 250 MHz) δ ppm: 7.39 (m, 1H), 7.04 (m, 1H), 4.60 (dt, J=8.7 Hz, 2.4 Hz, 2H), 3.16 (dt, J=9.1 Hz, 1.1 Hz, 2H).

The compound was synthesized from 5-amino-2-mercapto-1-methyl-1H-imidazole-4-carboxamide and 5,6-dibromo-2,3-dihydro-1-benzofuran following the experimental procedure detailed in Example 4. It was purified by flash chromatography on SiO₂ (5% MeOH/CH₂Cl₂) to yield an off-white solid that was slurred with Et₂O to furnish a white solid (yield: 13%). ¹H NMR (DMSO-d₆, 250 MHz) δ ppm: 7.48 (bs, 1H), 7.10 (bs, 1H), 6.79 (bs, 1H), 6.18 (bs, 2H), 5.81 (s, 1H), 4.52 (t, J₁=8.7 Hz, 2H), 3.33 (s, 3H), 3.14 (t, J₁=8.7 Hz, 2H). EI MS: m/z=369 (M+1).

### Example 98

### Preparation of 5-amino-2-{[6-(methylthio)-1,3-benzodioxol-5-yl]thio}-1-[3-(diethylamino)propyl]-1H-imidazole-4-carboxamide

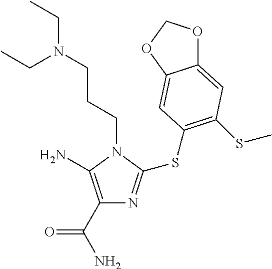

The compound was synthesized from 5-amino-1-[3-(diethylamino)propyl]-2-mercapto-1H-imidazole-4-carboxamide and 5-bromo-6-(methylthio)-1,3-benzodioxole following the experimental procedure detailed in Example 73. It was purified by flash chromatography on SiO₂ (5% MeOH/CH₂Cl₂) to yield an off-white solid that was slurred with Et₂O to give a white solid (yield: 22%). ¹H NMR (DMSO-d₆, 250 MHz) δ ppm: 7.08 (bs, 1H), 7.06 (s, 1H), 6.78 (bs, 1H), 6.33 (s, 1H), 6.26 (bs, 2H), 6.01 (s, 2H), 3.89 (m, 2H), 3.01 (m, 6H), 2.47 (s, 3H), 1.85 (m, 2H), 1.11 (m, 6H). EI MS: m/z=438 (M+1).

### Example 99

### Preparation of 5-amino-2-{[6-(methylthio)-2,3-dihydro-1-benzofuran-5-yl]thio}-methyl-1H-imidazole-4-carboxamide

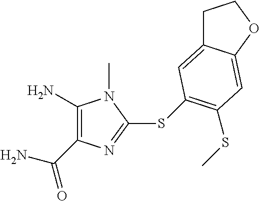

nBuLi (2.5 M in hexanes, 1.9 mL, 4.75 mmol) was added to a −78° C. cooled solution of 6-bromo-2,3-dihydro-1-benzofuran (0.86 g, 4.32 mmol) in THF (20 mL). The reaction mixture was stirred at low temperature for 30 min. Methyl disulfide (0.766 mL, 8.64 mmol) was added and mixture was stirred at low temperature for 1 h. The reaction mixture was poured into H₂O (20 mL) and extracted with EtOAc (2×20 mL). The organic layer was dried over Na₂SO₄ (anhydrous), filtered and concentrated. The crude residue was purified by flash chromatography on SiO₂ (0%→2% EtOAc/hexanes) to furnish 6-(methylthio)-2,3-dihydro-1-benzofuran (0.445 g, yellow oil, yield: 62%). ¹H NMR (CDCl₃, 250 MHz) δ ppm: 7.09 (m, 1H), 6.76 (m, 2H), 4.57 (dt, J=8.5 Hz, J=2.2 Hz, 2H), 3.16 (m, 2H), 2.46 (s, 3H).

NBS (0.471 g, 2.64 mmol) was added to a solution of 6-(methylthio)-2,3-dihydro-1-benzofuran (0.4 g, 2.4 mmol) in CH₃CN (20 mL). The reaction mixture was stirred at room temperature for 1 h, poured into H₂O (30 mL) and extracted with EtOAc (2×25 mL). The organic layer was dried over Na₂SO₄ (anhydrous), filtered and concentrated. The crude residue was purified by flash chromatography on SiO₂ (0%→2% EtOAc/hexanes) to furnish 5-bromo-6-(methylthio)-2,3-dihydro-1-benzofuran (1.3 g, yellow solid, yield: 84%). ¹H NMR (CDCl₃, 250 MHz) δ ppm: 7.31 (s, 1H), 6.61 (s, 1H), 4.59 (t, J=8.5 Hz, 2H), 3.18 (t, J=8.7 Hz, 2H), 2.43 (s, 3H).

The compound was synthesized from 5-amino-2-mercapto-1-methyl-1H-imidazole-4-carboxamide and 5-bromo-6-(methylthio)-2,3-dihydro-1-benzofuran following the experimental procedure detailed in Example 4. It was purified by flash chromatography on SiO₂ (3% MeOH/CH₂Cl₂/NH₃) to yield an off-white solid that was precipitated from Et₂O (yield: 10%). ¹H NMR (DMSO-d₆, 250 MHz) δ ppm: 7.01 (bs, 1H), 6.74 (m, 2H), 6.06 (s, 2H), 4.50 (t, J=7.4 Hz, 2H), 3.32 (s, 3H), 3.07 (t, J=7.9 Hz, 2H), 2.47 (s, 3H). EI MS: m/z=337 (M+1).

### Example 100

### Preparation of 5-amino-2-{[6-(ethylthio)-1,3-benzodioxol-5-yl]thio}-1-[3-(diethylamino)propyl]-1H-imidazole-4-carboxamide

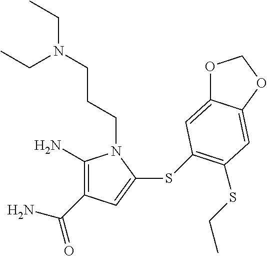

nBuLi (2.5 M in hexanes, 3.82 mL, 9.57 mmol) was added to a −78° C. cooled solution of 1-bromo-3,4-(methylenedioxy)benzene (1.75 g, 8.7 mmol) in THF (30 mL). The reaction mixture was stirred at low temperature for 30 min. Ethyl disulfide (2.14 mL, 17.41 mmol) was added and mixture was stirred at low temperature for 30 min. The reaction mixture was poured into H₂O (50 mL) and extracted with EtOAc (2×50 mL). The organic layer was dried over Na₂SO₄ (anhydrous), filtered and concentrated. The crude residue was purified by flash chromatography on SiO₂ (5% EtOAc/hexanes) to furnish 5-(ethylthio)-1,3-benzodioxole (1.57 g, yellow oil, yield: 99%). ¹H NMR (CDCl₃, 250 MHz) δ ppm: 6.95-6.70 (m, 3H), 5.96 (s, 2H), 2.84 (c, J=7.4 Hz, 2H), 1.32 (t, J=7.4 Hz, 3H).

NBS (1.61 g, 9.05 mmol) was added to a solution of 5-(ethylthio)-1,3-benzodioxole (1.5 g, 8.23 mmol) in CH₃CN (25 mL). The reaction mixture was stirred at room temperature. for 1 h, poured into H₂O (30 mL) and extracted with EtOAc (2×30 mL). The organic layer was dried over Na₂SO₄ (anhydrous), filtered and concentrated. The crude residue was purified by flash chromatography on SiO₂ (2% EtOAc/hexanes) to furnish 5-bromo-6-(ethylthio)-1,3-benzodioxole (1.38 g, yellow solid, yield: 64%). ¹H NMR (CDCl₃, 250 MHz) δ ppm: 7.04 (s, 1H), 6.87 (s, 1H), 5.97 (s, 2H), 2.88 (c, J=7.4 Hz, 2H), 1.3 (t, J=7.4 Hz, 3H).

The compound was synthesized from 5-amino-1-[3-(diethylamino)propyl]-2-mercapto-1H-imidazole-4-carboxamide and 5-bromo-6-(ethylthio)-1,3-benzodioxole following the experimental procedure detailed in Example 73. It was purified by flash chromatography on SiO₂ (4%→10% MeOH/CH₂Cl₂) to yield an off-white solid that was precipitated from Et₂O (yield: 40%). ¹H NMR (DMSO-d₆, 250 MHz) δ ppm: 7.08 (bs, 2H), 6.79 (s, 1H), 6.27 (bs, 2H), 6.11 (s, 1H), 3.81 (m, 2H), 2.91 (m, 3H), 1.64 (m, 3H), 1.19 (m, 4H), 0.91 (m, 9H). EI MS: m/z=452 (M+1).

### Example 101

### Human Hsp90α Fluorescence Polarization Assay

The aim of this study was to determine the binding ability of compounds of the invention to the N-terminal ATP binding site of Hsp90. For that purpose competition assays with fluorescently labeled Hsp90 binders were used (compounds of examples 37 and 41).

Compounds of examples 37 and 41 are structurally based on 8-(6-Iodo-benzo[1,3]dioxol-5-ylsulfanyl)-9-(3-isopropylamino-propyl)-9H-purin-6-ylamine, a known binder of the N-terminal ATP binding site of Hsp90 which has the ability of inhibiting the Hsp90 function (cf. Chiosis G. et al. “Identification of Potent Water Soluble Purine-Scaffold Inhibitors of the Heat Shock Protein 90”, J. Med. Chem., 2006, vol. 49, no. 1, p. 381). Compounds 37 and 41 keep the essential structural features of the known binder (which means that said compounds are able to bind to the ATP binding site) and incorporate a fluorescent probe which allows the detection of the binding by fluorescence polarization.

When the binders of examples 37 and 41 are used in the competition assay, in a first step said binders are allowed to interact with the N-terminal ATP binding site of Hsp90. Then the compounds of the invention are added and it is determined if they are able to bind to the N-terminal ATP binding site (which will imply that the binders of examples 37 and 41 have been replaced from the N-terminal ATP binding site). If after performing the assay, the binding between the tested compound of the invention and the Hsp90 is observed, this will be indicative that the compound of the invention has been bound to the ATP binding site of Hsp90 and, therefore, can inhibit the Hsp90.

### a) Materials and Methods

Once compounds of examples 37 and 41 were synthesized as reported above, they were dissolved at 238 μM and 1 mM in DMSO respectively (Sigma-Aldrich, cat. No. 154938). Hsp90α was purchased from Stressgen Bioreagents (cat. No. SPP-776; Victoria, Canada). The assay buffer contained 100 mM Tris·HCl, pH 7.4, 20 mM KCl, 6 mM MgCl₂. Before each use 5 μg/mL bovine serum albumin (BSA, Pierce, Rockford (Ill.), cat No. 23209), 0.25 mM TCEP (Sigma, cat No. 64706) and 0.01% NP40 (Nonidet P40 Substitute, Fluka, cat No. 74385) were freshly added (cf. Howes et al., “A fluorescence polarization assay for inhibitors of Hsp90”, Analytical Biochemistry, 2006, vol. 350, p. 202-213; Kim et al., “Development of a fluorescence polarization assay for the molecular chaperone Hsp90”, J. Biomol. Screen., 2004, vol. 9 (5)).

### b) Fluorescence Polarization Measurements

Fluorescence polarization measurements were performed on an Infinite F200 (Tecan, Austria). Measurements were taken in black 96-well microplates (Corning Costar No. 3650) in which both the excitation and the emission occurred from the top of the wells. In the Infinite F200, a xenon arc lamp provides excitation light that passes through a 485 nm (20-nm bandwidth) excitation filter and then a polarizer filter. A beam splitter filter directs the polarized excitation light into the well, and emitted fluorescence transmits back through the same beam-splitter filter, through a polarizer filter, and then through a 535 nm (25-nm bandwidth) emission filter for detection (Kim et al., supra). The read time per well was 20 μs, and the number of reads per well was 25.

### c) Assay Optimization for Example 37/Hsp90α and Example 41/Hsp90α

The compound of example 37 was used as a tracer by measuring changes on fluorescent polarization (mP) upon binding to Hsp90α. Binding isotherms between the compound of example 37 and Hsp90α were obtained by duplicate under the following conditions: each individual well in a 96-well assay plate contained 100 nM of the compound of example 37 and increasing concentrations (from 0 to 1.5 μM) of Hsp90α protein, in assay buffer containing the indicated TCEP, BSA and NP40 concentrations in a final volume of 100 μL. The plate was mixed thoroughly and measured at room temperature with an excitation wavelength at 485 nm and emission wavelength at 535 nm. Control wells contained assay buffer with either only protein at its maximum assay concentration, or tracer at 100 nM. (Kim et al., supra).

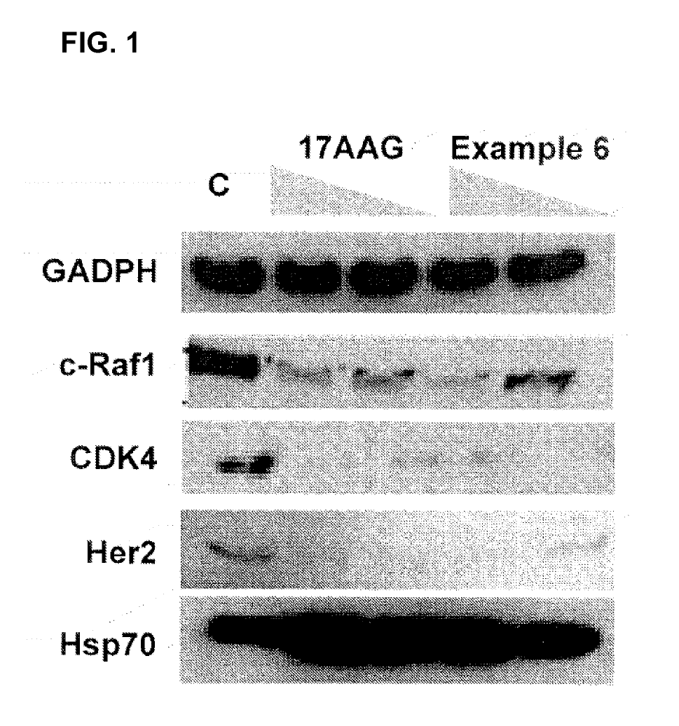

Data from experiments were fitted to a 1:1 binding model to determine the dissociation constant (K_{d}; FIG. 1). The tracer and Hsp90α had a K_{D} of 60 nM.

The compound of example 41 was also used as a tracer by measuring changes on fluorescent polarization (mP) upon binding to Hsp90α. Binding isotherms between the compound of example 41 and Hsp90α were obtained by duplicate under the following conditions: each individual well in a 96-well assay plate contained 200 nM of the compound of example 41 and increasing concentrations (from 0 to 1.5 μM) of Hsp90α protein, in assay buffer containing the indicated TCEP, BSA and NP40 concentrations in a final volume of 100 μL. The plate was mixed thoroughly and measured at room temperature with an excitation wavelength at 485 nm and emission wavelength at 535 nm. Control wells contained assay buffer with either only protein at its maximum assay concentration, or tracer at 200 nM. (Kim et al., supra).

Data from experiments were fitted to a 1:1 binding model to determine the dissociation constant. The tracer and Hsp90α had a K_{D} of 21 nM

d) Competition Assays with Example 37

Compounds were assayed over a three-fold dilution series in a 96-well plate format to determine IC₅₀ values. The serial dilution was performed in a separate 96-well plate (Thermowell 96 well PCR plate, Non-Treated Polypropylene, Corning Costar No. 6551) at 50 times final assay concentration in DMSO. (Howes et al, supra). 2 μl of compound was transferred to a black 96-well plate (Corning Costar No. 3650) and 98 μL of reaction solution (100 mM Tris·HCl, pH 7.4, 20 mM KCl, 6 mM MgCl₂, 5 μg/mL bovine serum albumin (BSA), 0.25 mM TCEP, 0.01% NP40, 50 nM the compound of example 37, 125 nM Hsp90α) was added per well. The plate was allowed to equilibrate for 1 h 30 min on a slow moving shaker at 20° C. in the dark. For each assay, background wells (buffer only), free tracer controls (50 nM the compound of example 37), and bound tracer controls (50 nM the compound of example 37, 125 nM Hsp90α) were included on each assay plate. After incubation, FP values were read, correlated to the fraction of tracer bound to Hsp90α, and plotted against log₁₀ values of competitor concentrations. IC₅₀ values are defined as the compound concentration at which 50% of bound compound of example 37 was displaced. (Table 1).

TABLE 1
Relative binding affinity values (IC₅₀) obtained from FP
measurements

| Example in patent | IC₅₀ (μM) |
|---|---|
| Example 6 | 0.88 |
| Example 10 | 1.70 |
| Example 27 | 200 |
| Example 33 | 0.50 |
| Example 44 | 3.20 |
| Example 48 | 2.76 |
| Example 51 | 3.10 |
| Example 55 | 2.80 |
| Example 69 | 200 |
| Example 72 | 3.00 |
| Example 73 | 15.0 |
| Example 75 | 56.0 |
| Example 77 | 4.50 |
| Example 79 | 12.5 |
| Example 81 | 4.00 |
| Example 83 | 8.00 |
| Example 85 | 5.30 |
| Example 86 | 3.40 |
| Example 87 | 0.90 |
| Example 88 | 0.60 |
| Example 89 | 4.80 |
| Example 97 | 5.00 |
| Example 98 | 2.00 |
| Example 99 | 13.0 |
| Example 100 | 1.45 |

These results confirm that the compounds of the invention are able to bind to the ATP binding site of the N-terminal domain of Hsp90 after replacing the compound previously bound (i.e., binder of example 37) at a nM/μM range. Therefore, it can be derived that the compounds of the invention can be used as inhibitors of the Hsp90.

### Example 102

### Determination of Influence of the Compounds of the Invention on Cell Proliferation by MTT Method

The objective of this study was to determine the influence of the compounds on cell proliferation of K-562, A-549, MCF-7, HeLa, WI-38 by MTT method. The compounds were tested over a three-fold dilution series in triplicate for the determination of EC₅₀ of their influence on cell proliferation.

The assay is based on the cleavage of the yellow tetrazolium salt MTT into purple formazan by metabolically active cells. The MTT formazan crystals are insoluble in aqueous solution, but may be solubilized by adding the 10% SDS, then incubating the plates overnight in a humidified atmosphere (e.g., 37° C., 5% CO₂). The solubilized formazan product can be photometrically quantitated using Tecan reader. An increase in the number of living cells results in an increase of total metabolic activity, which leads to a stronger color formation.

EC₅₀ values are defined as the compound concentration at which 50% of reaction product is formed.

### a) Assay Protocol

Test compounds were dissolved to 10 mM in 100% DMSO.

In the day of the experiment cells were seeded into all wells of 96-well plates. Serial dilution of compounds was performed in a separate 96-well plate at 20 times final assay concentration in cell culture medium (DMEM with 10% Fetal Bovine Serum). 5 μl of compound was transferred to 96-well plate containing seeded cells in cell culture medium (final total volume=100 μl). Cells were cultured in the presence of the respective test substances in the 96-well plate at 37° C. in a humidified 5% CO₂ incubator for a certain period of time (24, 72 or 120 hours). Then 5 μL of thiazolyl blue tetrazolium bromide (MTT) 5 mg/mL was added to the cells and the cells were reincubated 4 hours in a humidified 5% CO₂ incubator. Then 10% SDS was added into the each well. The reaction product was quantified by measuring the absorbance at 568 nm using a scanning multiwell spectrophotometer Tecan. The developed color and thereby the absorbance values directly correlate to the number of the living cells.

The data were analyzed using Microsoft Excel 2000, GraphPad Prism 3.03.

### c) Assay Reagents

Cell lines:

K 562 (human, chronic myelongenous leukemia),
A 549 (human, lung carcinoma),
MCF-7 (human, breast adenocarcinoma),
Hela (human, cervix, carcinoma, epitheloid),
WI-38 (human, embryonic lung)

The cell lines proceed from the collection of Institute of Cytology (Russian Academy of Sciences)

Materials:

96-well cell culture cluster (Costar Corning #3799)
175 cm² cell culture flask (Costar Corning #431079)
Cell culture medium RPMI 1640, DMEM, L-15
Fetal bovine serum (HyClone, Standard)

Stock solutions:

MTT (Thiazolyl Blue Tetrazolium Bromide) (Sigma # M5655)

### d) Instrumentation

Multichannel pipettes (Finnpipette, Thermo Labsystems) were used for liquid handling of all solutions. Safire (Tecan) plate reader was used for fluorescence detection of the amount of dye conversion in solution.

The results are included in Tables 2 and 3 below.

TABLE 2
EC₅₀ values (μM) for the example 6 of the present invention
on K-562, A-549, MCF-7, HeLa, WI-38 cell lines.

| Cell | Incubation | |
|---|---|---|
| line | time | Example 6 |
| K 562 | 1 day | >100 |
| | 3 days | 12.2 |
| | 5 days | 9.1 |
| A 549 | 1 day | >100 |
| | 3 days | 24.1 |
| | 5 days | 14.9 |
| MCF-7 | 1 day | >100 |
| | 3 days | 17.3 |
| | 5 days | 16.7 |
| HeLa | 1 day | >100 |
| | 3 days | 14.6 |
| | 5 days | 8.6 |
| WI-38 | 1 day | >100 |
| | 3 days | 27.7 |
| | 5 days | 19.5 |

TABLE 3
EC₅₀ values (μM) for the example 10 of the present invention
on A-549 and MCF-7cell lines.

| Cell | Incubation | Example |
|---|---|---|
| line | time | 10 |
| A 549 | 1 day | >100 |
| | 3 days | 13.3 |
| | 5 days | 16.2 |
| MCF-7 | 1 day | >100 |
| | 3 days | 10.1 |
| | 5 days | 11.7 |

The results show that the compounds of the invention are active towards several human cell lines what it means that they are promising anticancer agents to treat several types of cancer.

### Example 103

### Determination of Influence of Compounds on Cell Proliferation by AlamarBlue Method

### Cell Culture

The human colon cancer cell line HCT-116 and the human breast cell lines MCF-7 and SKBr3 were from the American Type Culture Collection (ATCC; CCL-247, HTB-22 and HTB-30, respectively).

The HCT-116 and SKBr3 cell lines were maintained in DMEM GlutaMAX (Invitrogen) supplemented with 10% fetal calf serum and the MCF-7 cell line was maintained in RPMI1640 GlutaMAX (Invitrogen) containing 1 mM sodium pyruvate and 10% fetal calf serum. Cells were grown in a humidified incubator at 37° C. in 5% CO₂

### AlamarBlue Assay

Cells were plated in 96-well plates (Costar, 3596) at a density of 6000 cells/well in 50 μL medium 24 h before addition of the compounds. Compounds were then added in 8 concentrations, each in triplicate (dilution series of 1 to 3). To do so, a compound-dilution plate at 200× the screening concentrations was prepared in DMSO 100% (Sigma, 154938). Then, 1.7 μL of the 200× diluted compounds were transferred to a new plate and 170 μL of media were added and mixed with the pipette (plate at 2×). Columns 11 and 12 of this plate (controls) were filled with 1% DMSO in cell media (i.e. 1.7 μL DMSO and 170 μL cell media). Finally, 50 μL of this intermediate plate at 2× were transferred to the plate containing the seeded cells and slowly shaken manually. Cells were grown as described above. AlamarBlue viability assay (Biosource, Invitrogen, DAL1100) was performed 72 hours later following manufacturer's protocol. In brief, AlamarBlue diluted in media was added to cells to have a 5% solution (a 2× solution was prepared in media and 100 μL of this solution were transferred to each well, i.e. final volume of 200 μL). Cells were incubated at 37° C., 3 hours and at room temperature for an additional 30 min. Cells with no compound and, cells with no compound and lysed with triton X-100 (30 μL/well added 10 min before treatment with AlamarBlue) were used as controls (6 replicates of each). Fluorescence was monitored at 530 nm excitation and 590 nm emission wavelengths. Results were quantified using ELx800 Universal Microplate Reader (Bio-Tek Instruments, Inc.). EC₅₀ were calculated as the dose of compound required to inhibit cell growth by 50%.

TABLE 4
Cell growth inhibition values (EC₅₀) obtained with AlamarBlue
method with HCT-116, SKBr3 and MCF-7 cell lines.

| | |
|---|---|
| Example in patent | EC₅₀ |

HCT-116

| | |
|---|---|
| Example 10 | 18 |
| Example 6 | 23.7 |
| Example 33 | 3.0 |
| Example 48 | 33 |
| Example 55 | 32 |
| Example 73 | 78 |
| Example 77 | 39.2 |
| Example 79 | 91.5 |
| Example 86 | 92 |
| Example 87 | 1 |
| Example 83 | 26.1 |
| Example 92 | 21 |
| Example 93 | 30.5 |
| Example 94 | 2.1 |
| Example 95 | 83 |
| Example 97 | 70 |
| Example 98 | 6 |
| Example 99 | 85 |
| Example 100 | 1.2 |

SKBr3

| | |
|---|---|
| Example 10 | 24-30 |
| Example 72 | 8 |
| Example 77 | 39.2 |
| Example 81 | 23 |
| Example 89 | 18 |
| Example 87 | 4.6 |
| Example 88 | 1.1 |
| Example 97 | 41 |
| Example 98 | 1.9 |
| Example 99 | 55 |

MCF-7

| | |
|---|---|
| Example 6 | 15.9 |
| Example 33 | 3.0 |
| Example 48 | 43 |
| Example 55 | 35 |
| Example 44 | 18.8 |

### Example 104

### Determination of Biomarkers in Cell Cultures

The aim of this study was to determine the influence of example 6 on the levels of pharmacodynamic biomarkers such as c-Raf-1, Cdk4, Her2 and Hsp70, indicative that the effect observed in cell viability is mediated through inhibition of Hsp90. GADPH was used as a control of protein loading.

As described in the literature, Hsp90 inhibitors such as 17AAG promotes degradation of its client proteins and overexpression of the co-chaperone Hsp70 (cf. Banerji U. et al. “Pharmacokinetic-PHarmacodynamic Relationships for the Heat Shock Protein 90 Molecular Chaperone Inhibitor 17-Allylamino, 17-Demethoxygeldanamycin in Human Ovarian Cancer Xenograft Models”, Clin. Can. Res., 2005, vol. 11, no. 19, p. 7023).

MCF7 cells (1×10⁶) were seeded on tissue culture dishes and allowed to grow overnight at 37° C. with 5% CO₂. Cells were treated with example 6 compound (at 33 and 100 μM) and the known Hsp90 inhibitor 17AAG (at 0, 33 and 1 μM). 0.5% DMSO was used as control. Seventy-two hours later, cells were collected, rinsed with PBSx1 and lysed in 200 μL ice-cold lysis buffer containing 50 mM Tris-HCl pH 8, 150 mM NaCl, 1% NP40, 0.1% SDS, 1 mM DTT, 1 mM EDTA, 1 mM PMSF, 20 μg/ml leupeptin and 1 μg/ml pepstatin. Protein concentration was measured by the Bradford method (Bio-Rad Protein Assay, Bio-Rad). Samples were adjusted to equal protein concentrations and then mixed with Laemmli sample buffer. Thirty μg of each whole cell lysate were resolved in 12% SDS-PAGE. Proteins were transferred from the gel to nitrocellulose membranes (Hybond-ECL, Amersham Biosciences), which were probed with anti-GAPDH antibody (sc-25778, Santa Cruz Biotechnology) diluted 1:500, anti-Hsp70 antibody (sc-66048, Santa Cruz Biotechnology) diluted 1:500, anti-c-Raf-1 antibody (sc-133 HRP, Santa Cruz Biotechnology) diluted 1:600, anti-CDK4 antibody (sc-260 HRP, Santa Cruz Biotechnology) diluted 1:500 and anti-HER-2 antibody (sc-31154 HRP, Santa Cruz Biotechnology) diluted 1:250 for 1 h at room temperature. Following appropriate secondary antibody incubation for 1 h at room temperature (anti-rabbit IgG-HRP, NA934V, Amersham Biosciences, diluted 1:8000; anti-mouse IgG-HRP, sc-2061, Santa Cruz Biotechnology, diluted 1:2500 and, anti-goat IgG-HRP, sc-31154, Santa Cruz Biotechnology, diluted 1:3500), the specific protein signals were detected using the ECL-Plus Western blotting detection system (GE Healthcare) according to the manufacturer's instructions. The molecular weights were estimated using pre-stained markers (SeeBlue Plus2 Pre-Stained Standard, LC5925, Invitrogen).

17AAG was acquired from Sigma-Aldrich (#A8476)

The results obtained for the compound of example 6 (FIG. 1) shows that there is a specific interaction with hsp90 target in cells. This finding can be extrapolated to the remaining compounds of the invention since, as it has been mentioned above, the amide group and the fixed nitrogen participate in the binding through direct and/or water mediated hydrogen bonds which are essential for the potency and the proper orientation of the molecule.

1. A compound of general formula (I), 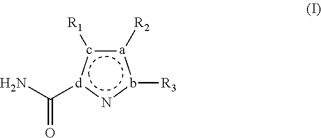
wherein
one of the a, b, c or d members is a nitrogen atom and the remaining members are carbon atoms,
R₁ is hydrogen or a radical selected from the group consisting of: —NR₄R₅; —COR₆; —COOR₆; —C(O)NR₄R₅; —R₉PO(OR₁₀)₂; —NHSO₂—R₁₁; (C₁-C₈)alkyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₁-C₈)-alkyl-Cy; (C₂-C₈)alkenyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₂-C₈)-alkenyl-Cy; (C₂-C₈)alkynyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₂-C₈)-alkynyl-Cy; and a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₂ is a radical selected from the group consisting of: (C₁-C₈)alkyl; (C₂-C₈)alkenyl; and (C₂-C₈)alkynyl; said radicals being optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, —NR₄R₅, and a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₃ is a radical selected from the group consisting of: —S—R₁₄ and —CH₂—R₁₅;
Cy is a known ring system with 1-4 rings, wherein each one of the rings forming said ring system has from 3 to 7 members, each member independently selected from C, N, O, S, CH, CH₂, and NH, the ring being saturated, partially unsaturated or aromatic, and optionally being substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₁₄ is a C-radical which is a known ring system with 1-4 rings, wherein each one of the rings:
has 3-7 members, each member independently selected from C, N, O, S, CH, CH₂, NH,
is saturated, partially unsaturated or aromatic, and
is isolated or partially/totally fused
being each ring forming part of the ring system optionally substituted by at least one radical selected from the group consisting of: halogen, nitro, cyano, —CF₃, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₁₅ is a radical which is a known ring system with 2-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, —NR₄R₅, and —CF₃; and
R₄ and R₅, are radicals, same or different, independently selected from the group consisting of: hydrogen; —COR₆; —COOR₆; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, amino; (C₂-C₈)alkynyl optionally substituted by at least one radical selected from the group consisting of: halogen, nitro, cyano, and amino; and radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is isolated, partially/totally fused;
being each ring forming part of the ring system optionally substituted by at least one radical selected from the group consisting of: halogen, nitro, cyano, amino, and (C₁-C₈)alkyl;
R₇ and R₉ are biradicals, same or different, independently selected from the group consisting of: (C₁-C₈)alkyl; (C₂-C₈)alkenyl; and (C₂-C₈)alkynyl; being said radicals optionally substituted by at least one radical selected from the group consisting of: halogen, nitro, cyano, amino, and a known 5- or 6-membered aromatic ring wherein all the members are carbon atoms or wherein from 1 to 4 of the members are selected from N, O, and S, and the remaining members are carbon atoms, being the aromatic ring optionally substituted by at least one radical selected from the group consisting of: halogen, nitro, cyano, amino, (C₁-C₈)alkyl, (C₂-C₈)alkenyl, —OR₆, —COR₆, —COOR₆, —OC(O)R₆, —C(O)NR₄R₅, —S—R₁₁, —SO—R₁₁, —SO₂—R₁₁, —NHSO₂—R₁₁, and, —SO₂—NR₁₂R₁₃; and
R₆, R₈, R₁₀, R₁₁, R₁₂, and R₁₃, are radicals, same or different, independently selected from the group consisting of: hydrogen; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, and amino; (C₂-C₈)alkynyl optionally substituted by at least one radical selected from the group consisting of: halogen, nitro, cyano, and amino; and a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, and amino.

2. The compound according to claim 1,
wherein
one of the a, b, or c members is a nitrogen atom and the remaining members are carbon atoms,
R₁ is hydrogen or a radical selected from the group consisting of: —NR₄R₅; —NHSO₂—R₁₁; (C₁-C₈)alkyl optionally substituted by at least one radical selected from the group consisting of: —COOR₆, —OC(O)R₆, —C(O)NR₄R₅, and —NR₄R₅, -Cy;
R₂ is a radical selected from the group consisting of: (C₁-C₈)alkyl; (C₂-C₈)alkenyl; and (C₂-C₈)alkynyl; said radicals being optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, —NR₄R₅, and a radical which is a known ring system with 1-2 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, (C₁-C₈)alkyl, —OR₆, and —NR₄R₅;
R₃ is a radical selected from the group consisting of: —S—R₁₄ and —CH₂—R₁₅;
Cy is a known ring system with 1-2 rings, wherein each one of the rings forming said ring system has from 3 to 7 members, each member independently selected from O, N, O, S, CH, CH₂, and NH, the ring being saturated, partially unsaturated or aromatic, and optionally being substituted by at least one radical selected from the group consisting of: halogen, (C₁-C₈)alkyl, —OR₆, and —NR₄R₅;
R₁₄ is a C-radical which is a known ring system with 1-4 rings, wherein each one of the rings:
has 3-7 members, each member independently selected from O, N, O, S, CH, CH₂, NH,
is saturated, partially unsaturated or aromatic, and
is isolated or partially/totally fused
being each ring forming part of the ring system optionally substituted by at least one radical selected from the group consisting of: halogen, nitro, cyano, (C₁-C₈)alkyl, —CF₃, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₁₅ is a radical which is a known ring system with 2-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, (C₁-C₈)alkyl, —CF₃, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —HSO₂—R₁₁, —SO₂—NR₁₂R₁₃, and —NR₄R₅; and
R₄ and R₅, are radicals, same or different, independently selected from the group consisting of: hydrogen; —COR₆; —COOR₆; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, amino; (C₂-C₈)alkynyl optionally substituted by at least one radical selected from the group consisting of: halogen, nitro, cyano, and amino; and radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is isolated, partially/totally fused;
being each ring forming part of the ring system optionally substituted by at least one radical selected from the group consisting of: halogen, nitro, cyano, amino, and (C₁-C₈)alkyl;
R₇ and R₉ are biradicals, same or different, independently selected from the group consisting of: (C₁-C₈)alkyl; (C₂-C₈)alkenyl; and (C₂-C₈)alkynyl; being said radicals optionally substituted by at least one radical selected from the group consisting of: halogen, and amino; and
R₆, R₈, R₁₀, R₁₁, R₁₂, and R₁₃, are radicals, same or different, independently selected from the group consisting of: hydrogen; (C₁-C₈)alkyl optionally substituted by at least one radical selected from the group consisting of: phenyl, halogen, nitro, cyano, and amino; (C₂-C₈)alkenyl optionally substituted by at least one radical selected from the group consisting of: halogen, nitro, cyano, and amino; (C₂-C₈)alkynyl optionally substituted by at least one radical selected from the group consisting of: halogen, nitro, cyano, and amino; and a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, and amino.

3. The compound according to claim 2, wherein
one of the a or b members is a nitrogen atom and the remaining members are carbon atoms;
R₁ is selected from —NHSO₂—R₁₁ and —NHR₅;
R₂ is a radical selected from the group consisting of: —R₇NHR₈; (C₁-C₈)alkyl; (C₂-C₈)alkenyl; (C₂-C₈)alkynyl; said radical being optionally substituted by at least one radical selected from the group consisting of: hydroxyl, halogen, nitro, cyano, —NH₂, —NH(C₁-C₈)alkyl, —COOH, —OC(O)(C₁-C₈)alkyl, —NR₄R₅, and a radical which is a known ring system with 1 ring, said ring
having 3-7 members, each member independently selected from C, N, O, S, CH, CH₂, NH,
is saturated, partially unsaturated or aromatic,
being the ring system optionally substituted by at least one radical selected from the group consisting of: halogen, (C₁-C₈)alkyl, and —OR₆;
R₃ is a radical selected from the group consisting of: —S—R₁₄ and —CH₂—R₁₅;
R₄, R₅ is hydrogen or a radical selected from the group consisting of: (C₁-C₈)alkyl optionally substituted by at least one radical selected from the group consisting of: phenyl, halogen, nitro, cyano, and —NH₂; (C₂-C₈)alkenyl optionally substituted by at least one radical selected from the group consisting of: halogen, nitro, cyano, and —NH₂; —COR₆; and a known ring system consisting of one ring which is aromatic, has from 5-6 members, and each member is independently selected from C, N, O, S, CH, CH₂, NH, being the aromatic ring optionally substituted by at least one radical selected from the group consisting of: halogen, nitro, cyano, —NH₂, and (C₁-C₈)alkyl;
R₆ is a radical selected from the group consisting of: (C₁-C₈)alkyl optionally substituted by one phenyl radical; a known ring system consisting of one ring which is saturated, has from 3-7 members, and each member is independently selected from C, N, O, S, CH, CH₂, NH; and a known ring system consisting of one ring which is aromatic, has from 5-6 members, and each member is independently selected from C, N, O, S, CH, CH₂, NH, being the ring system optionally substituted by at least one radical selected from the group consisting of: halogen, nitro, cyano, —NH₂, and (C₁-C₈)alkyl;
R₁₄ is a C-radical which is a known ring system with 1-4 rings, wherein each one of the rings:
has 5-6 members, each member independently selected from C, N, O, S, CH, CH₂, NH,
is saturated, partially unsaturated or aromatic, and
is isolated or partially/totally fused;
being each ring forming part of the ring system optionally substituted by at least one radical selected from the group consisting of: hydroxyl, halogen, nitro, cyano, —NH₂, —COOH, (C₁-C₈)alkyl, (C₁-C₈)alkenyl, (C₁-C₄)alkoxy, —S—R₁₁, —SO₂—R₁₁, and —CF₃;
R₁₅ is a radical which is a known ring system with 2-4 rings, wherein each one of the rings forming said ring system
has 5-6 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: hydroxyl, halogen, nitro, cyano, —NH₂, —COOH, (C₁-C₈)alkyl, (C₁-C₈)alkenyl, (C₁-C₄)alkoxy, —S—R₁₁, —SO₂—R₁₁, and —CF₃; and
R₁₁ is (C₁-C₈)alkyl.

4. The compound according to claim 3, wherein
one of the a or b members is a nitrogen atom and the remaining members are carbon atoms;
R₁ is —NHR₅;
R₂ is a radical selected from the group consisting of: (C₁-C₈)alkyl; (C₂-C₈)alkenyl; (C₂-C₈)alkynyl; said radical being optionally substituted by at least one radical selected from the group consisting of: hydroxyl, halogen, nitro, cyano, —NH₂, —NH(C₁-C₈)alkyl, —COOH, —OC(O)(C₁-C₈)alkyl, and phenyl;
R₃ is a radical selected from the group consisting of: —S—R₁₄ and —CH₂—R₁₅;
R₅ is hydrogen or a radical selected from the group consisting of: (C₁-C₈)alkyl optionally substituted by at least one radical selected from the group consisting of: halogen, nitro, cyano, and —NH₂; (C₂-C₈)alkenyl optionally substituted by at least one radical selected from the group consisting of: halogen, nitro, cyano, and —NH₂; —COR₆; and a known ring system consisting of one ring which is aromatic, has from 5-6 members, and each member is independently selected from C, N, O, S, CH, CH₂, NH, being the aromatic ring optionally substituted by at least one radical selected from the group consisting of: halogen, nitro, cyano, —NH₂, and (C₁-C₈)alkyl;
R₆ is a radical selected from the group consisting of: (C₁-C₈)alkyl; a known ring system consisting of one ring which is saturated, has from 3-7 members, and each member is independently selected from C, N, O, S, CH, CH₂, NH; and a known ring system consisting of one ring which is aromatic, has from 5-6 members, and each member is independently selected from C, N, O, S, CH, CH₂, NH; being the radical optionally substituted by at least one radical selected from the group consisting of: halogen, nitro, cyano, —NH₂, and (C₁-C₈)alkyl;
R₁₄ is a C-radical which is a known ring system with 1-4 rings, wherein each one of the rings:
has 5-6 members, each member independently selected from C, N, O, S, CH, CH₂, NH,
is saturated, partially unsaturated or aromatic, and
is isolated or partially/totally fused;
being each ring forming part of the ring system optionally substituted by at least one radical selected from the group consisting of: hydroxyl, halogen, nitro, cyano, —NH₂, —COOH, (C₁-C₈)alkyl, and (C₁-C₈)alkenyl; and
R₁₅ is a radical which is a known ring system with 2-4 rings, wherein each one of the rings forming said ring system
has 5-6 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: hydroxyl, halogen, nitro, cyano, —NH₂, —COOH, (C₁-C₈)alkyl, and (C₁-C₈)alkenyl.

5. The compound according to claim 4, wherein R₃ is the radical —S—R₁₄.

6. The compound according to claim 4, wherein R₁₄ is a known ring system consisting of one ring.

7. The compound according to claim 4, wherein R₁₄ is a known ring system consisting of two or three rings, the rings being totally fused.

8. The compound according to claim 4, wherein R₃ is the radical —CH₂—R₁₅.

9. The compound according claim 8, wherein R₁₅ is a known ring system consists of two or three rings, the rings being totally fused.

10. The compound according to claim 4, wherein R₁₄ or R₁₅ are selected from the group consisting of: 6-iodobenzo[1,3]dioxol, benzo[1,3]dioxol, trifluoromethylphenyl, and 2,5-dimethoxyphenyl.

11. The compound according to claim 4, wherein R₂ is selected from the group consisting of: butyl, pentylamine, and (3-isopropylamine)propyl.

12. The compound according to claim 3, wherein
the a member is a nitrogen atom and the remaining members are carbon atoms;
R₂ is a radical selected from the group consisting of: (C₁-C₈)alkyl optionally substituted by at least one radical selected from the group consisting of: —NH₂, —NH(C₁-C₄)alkyl, —NR₄R₅ and a radical which is a known ring system with 1 saturated ring, the ring having 6 members independently selected from N, O, and CH₂;
R₄ is (C₁-C₄)alkyl;
R₅ is hydrogen or a radical selected from the group consisting of: (C₁-C₄)alkyl optionally substituted by one phenyl radical; —C(O)(C₁-C₄)alkyl optionally substituted by one phenyl radical; —C(O)cyclopropyl; and —SO₂CH₃;
R₃ is a radical selected from the group consisting of: —S—R₁₄ and —CH₂—R₁₅
R₁₄ is a C-radical which is a known ring system with 1-2 rings, wherein each one of the rings:
has 5-6 members, each member independently selected from C, N, O, S, CH, CH₂, NH,
is saturated, partially unsaturated or aromatic, and is partially/totally fused;
being each ring forming part of the ring system optionally substituted by at least one radical selected from the group consisting of: halogen, (C₁-C₄)alkyl (C₁-C₄)alkenyl, (C₁-C₄)alkoxy, —S—CH₃, —SO₂—CH₃, and —CF₃; and
R₁₅ is a radical which is a known ring system with 2 rings totally fused, wherein each one of the rings:
has 5-6 members, each member independently selected from C, O, CH, and, CH₂, and
is saturated, partially unsaturated or aromatic;
being each ring forming part of the ring system optionally substituted by at least one radical selected from the group consisting of: halogen, (C₁-C₄)alkyl (C₁-C₄)alkenyl, (C₁-C₄)alkoxy, —S—CH₃, —SO₂—CH₃, and —CF₃.

13. The compound according to claim 3, wherein
the b member is a nitrogen atom and the remaining members are carbon atoms;
R₂ is (C₁-C₄)alkyl;
R₅ is hydrogen or a radical selected from the group consisting of: —C(O)(C₁-C₄)alkyl; and —SO₂—CH₃;
R₃ is —CH₂—R₁₅;
R₁₅ is a C-radical which is a known ring system with 2 rings totally fused, wherein each one of the rings:
has 5-6 members, each member independently selected from C, O, CH, and CH₂,
is saturated, partially unsaturated or aromatic, and
being each ring forming part of the ring system optionally substituted by one one halogen radical.

14. The compound according to any of the preceding claims which is selected from the group consisting of:
5-amino-1-(5-amino-pentyl)-2-(6-iodo-benzo[1,3]dioxol-5-ylsulfanyl)-1H-imidazole-4-carboxamide;
5-amino-1-butyl-2-(6-iodo-benzo[1,3]dioxol-5-ylsulfanyl)-1H-imidazole-4-carboxamide;
5-amino-2-(benzo[d][1,3]dioxol-5-ylthio)-1-butyl-1H-imidazole-4-carboxamide;
5-amino-1-(5-aminopentyl)-2-(benzo[d][1,3]dioxol-5-ylthio)-1H-imidazole-4-carboxamide;
5-acetamido-2-(benzo[d][1,3]dioxol-5-ylthio)-1-butyl-1H-imidazole-4-carboxamide;
2-(4-(trifluoromethyl)phenylthio)-5-amino-1-butyl-1H-imidazole-4-carboxamide;
5-amino-1-butyl-2-(2,5-dimethoxy-phenylsulfanyl)-1H-imidazole-4-carboxamide;
5-acetylamino-1-butyl-2-(6-iodo-benzo[1,3]dioxol-5-ylsulfanyl)-1H-imidazole-4-carboxamide;
5-amino-2-(6-iodo-benzo[1,3]dioxol-5-ylsulfanyl)-1-(3-isopropylamino-propyl)-1H-imidazole-4-carboxamide;
5-amino-2-(benzo[d][1,3]dioxol-5-ylthio)-1-butyl-1H-imidazole-4-carboxamide;
5-amino-1-butyl-2-(7-chloro-benzothiazol-2-ylsulfanyl)-1H-imidazole-4-carboxamide;
5-amino-1-butyl-2-(4-phenyl-thiazol-2-ylsulfanyl)-1H-imidazole-4-carboxamide;
5-amino-2-(7-chloro-benzothiazol-2-ylsulfanyl)-1-methyl-1H-imidazole-4-carboxamide;
5-amino-2-(7-chloro-benzothiazol-2-ylsulfanyl)-1-(3-isopropylamino-propyl)-1H-imidazole-4-carboxamide;
5-amino-1-(3-isopropylamino-propyl)-2-(1-isopropyl-1H-benzoimidazol-2-ylsulfanyl)-1H-imidazole-4-carboxamide;
5-amino-2-(3-chloro-5-trifluoromethyl-pyridin-2-ylsulfanyl)-1-(3-isopropylamino-propyl)-1H-imidazole-4-carboxamide;
5-amino-1-(3-isopropylamino-propyl)-2-(naphthalen-2-ylsulfanyl)-1H-imidazole-4-carboxamide;
5-amino-1-(3-isopropylamino-propyl)-2-(4-phenyl-thiazol-2-ylsulfanyl)-1H-imidazole-4-carboxamide;
5-(cyclopropanecarbonyl-amino)-2-(6-iodo-benzo[1,3]dioxol-5-ylsulfanyl)-1-(3-isopropylamino-propyl)-1H-imidazole-4-carboxamide;
5-acetylamino-2-(7-chloro-benzothiazol-2-ylsulfanyl)-1-(3-isopropylamino-propyl)-1H-imidazole-4-carboxamide;
5-acetylamino-2-(6-iodo-benzo[1,3]dioxol-5-ylsulfanyl)-1-(3-isopropylamino-propyl)-1H-imidazole-4-carboxamide;
2-(6-Iodo-benzo[1,3]dioxol-5-ylsulfanyl)-1-methyl-5-phenylacetylamino-1H-imidazole-4-carboxamide;
2-(5-iodobenzo[d][1,3]dioxol-6-ylthio)-1-methyl-5-(phenethylamino)-1H-imidazole-4-carboxamide;
5-amino-2-[(6-bromo-1,3-benzodioxol-5-yl)thio]-1-methyl-1H-imidazole-4-carboxamide;
5-amino-2-[(6-isopropyl-1,3-benzodioxol-5-yl)thio]-1-methyl-1H-imidazole-4-carboxamide;
5-amino-1-methyl-2-[(6-vinyl-1,3-benzodioxol-5-yl)thio]-1H-imidazole-4-carboxamide;
5-amino-2-[(6-ethoxy-1,3-benzodioxol-5-yl)thio]-1-methyl-1H-imidazole-4-carboxamide;
5-amino-1-methyl-2-{[6-(methylthio)-1,3-benzodioxol-5-yl]thio}-1H-imidazole-4-carboxamide;
5-amino-1-methyl-2-{[6-(methylsulfonyl)-1,3-benzodioxol-5-yl]thio}-1H-imidazole-4-carboxamide;
5-amino-2-[(6-iodo-1,3-benzodioxol-5-yl)thio]-1-methyl-1H-imidazole-4-carboxamide;
5-amino-2-[(6-bromo-1,3-benzodioxol-5-yl)thio]-1-(3-morpholin-4-ylpropyl)-1H-imidazole-4-carboxamide;
5-amino-2-[(6-iodo-1,3-benzodioxol-5-yl)thio]-1-(3-morpholin-4-ylpropyl)-1H-imidazole-4-carboxamide;
5-amino-2-[(6-iodo-1,3-benzodioxol-5-yl)thio]-1-[3-(diethylamino)propyl]-1H-imidazole-4-carboxamide;
5-amino-2-[(6-bromo-1,3-benzodioxol-5-yl)thio]-1-[3-(diethylamino)propyl]-1H-imidazole-4-carboxamide;
2-[(6-bromo-1,3-benzodioxol-5-yl)thio]-1-methyl-5-[(methylsulfonyl)amino]-1H-imidazole-4-carboxamide;
5-(acetylamino)-2-[(6-bromo-1,3-benzodioxol-5-yl)thio]-1-methyl-1H-imidazole-4-carboxamide;
4-amino-1-(6-bromo-benzo[1,3]dioxol-5-ylmethyl)-5-methyl-1H-pyrazole-3-carboxamide;
4-(acetylamino)-1-[(6-bromo-1,3-benzodioxol-5-yl)methyl]-5-methyl-1H-pyrazole-3-carboxamide;
1-[(6-bromo-1,3-benzodioxol-5-yl)methyl]-5-methyl-4-(propionylamino)-1H-pyrazole-3-carboxamide;
1-[(6-bromo-1,3-benzodioxol-5-yl)methyl]-5-methyl-4-[(methylsulfonyl)amino]-1H-pyrazole-3-carboxamide;
4-amino-1-[(6-iodo-1,3-benzodioxol-5-yl)methyl]-5-methyl-1H-pyrazole-3-carboxamide;
5-amino-2-[(6-bromo-2,3-dihydro-1-benzofuran-5-yl)thio]-1-methyl-1H-imidazole-4-carboxamide;
5-amino-2-{[6-(methylthio)-1,3-benzodioxol-5-yl]thio}-1-[3-(diethylamino) propyl]-1H-imidazole-4-carboxamide.
5-amino-2-{[6-(ethylthio)-1,3-benzodioxol-5-yl]thio}-1-[3-(diethylamino) propyl]-1H-imidazole-4-carboxamide; and
5-amino-2-{[6-(methylthio)-2,3-dihydro-1-benzofuran-5-yl]thio}1-methyl-1H-imidazole-4-carboxamide.

15. A pharmaceutical composition comprising a therapeutically effective amount of a compound of formula (I) as defined in any of claims 1 to 14, together with the appropriate amounts of pharmaceutically acceptable excipients, carriers or mixtures thereof.

16. A compound of formula (I) as defined in any of the claims 1 to 14, or a pharmaceutical composition according to claim 12, for use as a medicament.

17. Use of a compound of formula (I), 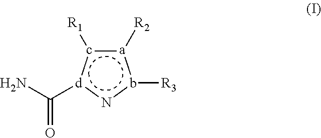
wherein
one of the a, b, c or d members is a nitrogen atom and the remaining members are carbon atoms;
R₁ is hydrogen or a radical selected from the group consisting of: —NR₄R₅; —COR₆; —COOR₆; —C(O)NR₄R₅; —R₉PO(OR₁₀)₂; —NHSO₂—R₁₁; (C₁-C₈)alkyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₁-C₈)-alkyl-Cy; (C₂-C₈)alkenyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₂-C₈)-alkenyl-Cy; (C₂-C₈)alkynyl optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅; —(C₂-C₈)-alkynyl-Cy; and a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₂ is a radical selected from the group consisting of: (C₁-C₈)alkyl; (C₂-C₈)alkenyl; and (C₂-C₈)alkynyl; said radicals being optionally substituted by at least one radical selected from the group consisting of: halogen, nitro, cyano, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, —NR₄R₅, and a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₃ is a radical selected from the group consisting of: —S—R₁₄ and —CH₂—R₁₅;
Cy is a known ring system with 1-4 rings, wherein each one of the rings forming said ring system has from 3 to 7 members, each member independently selected from C, N, O, S, CH, CH₂, and NH, the ring being saturated, partially unsaturated or aromatic, and optionally being substituted by at least one radical selected from the group consisting of: halogen, nitro, cyano, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₁₄ is selected from the group consisting of: C₁-C₈ alkyl; C₂-C₈ alkenyl; C₂-C₈ alkynyl; and a C-radical which is a known ring system with 1-4 rings, wherein each one of the rings:
has 3-7 members, each member independently selected from C, N, O, S, CH, CH₂, NH,
is saturated, partially unsaturated or aromatic, and
is isolated or partially/totally fused
being each ring forming part of the ring system optionally substituted by at least one radical selected from the group consisting of: halogen, nitro, cyano, CF₃, (C₁-C₈)alkyl, (C₂-C₈)alkenyl, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₁₅ is a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, (C₁-C₈)alkyl, (C₂-C₈)alkenyl, CF₃, —OR₆, —COR₆, —COOR₆, —OC(O)R₆, —C(O)NR₄R₅, —R₇NHR₈, —R₉PO(OR₁₀)₂, —S—R₁₁, —SO—R₁₁, —SO₂—R₁₁, —NHSO₂—R₁₁, —SO₂—NR₁₂R₁₃, and —NR₄R₅;
R₄ and R₅, are radicals, same or different, independently selected from the group consisting of: hydrogen; —COR₆; —COOR₆; (C₁-C₈)alkyl optionally substituted by at least one radical selected from the group consisting of: halogen, nitro, cyano, phenyl, and amino; (C₂-C₈)alkenyl optionally substituted by at least one radical selected from the group consisting of: halogen, nitro, cyano, amino; (C₂-C₈)alkynyl optionally substituted by at least one radical selected from the group consisting of: halogen, nitro, cyano, and amino; and radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is isolated, partially/totally fused;
being each ring forming part of the ring system optionally substituted by at least one radical selected from the group consisting of: halogen, nitro, cyano, amino, and (C₁-C₈)alkyl;
R₇ and R₉ are biradicals, same or different, independently selected from the group consisting of: (C₁-C₈)alkyl; (C₂-C₈)alkenyl; and (C₂-C₈)alkynyl; being said radicals optionally substituted by at least one radical selected from the group consisting of: halogen, nitro, cyano, amino, and a known 5- or 6-membered aromatic ring wherein all the members are carbon atoms or wherein from 1 to 4 of the members are selected from N, O, and S, and the remaining members are carbon atoms, being the aromatic ring optionally substituted by at least one radical selected from the group consisting of: halogen, nitro, cyano, amino, (C₁-C₈)alkyl, (C₂-C₈)alkenyl, —OR₆, —COR₆, —COOR₆, —OC(O)R₆, —C(O)NR₄R₅, —S—R₁₁, —SO—R₁₁, —SO₂—R₁₁, —NHSO₂—R₁₁, and, —SO₂—NR₁₂R₁₃; and
R₆, R₈, R₁₀, R₁₁, R₁₂, and R₁₃, are radicals, same or different, independently selected from the group consisting of: hydrogen; (C₁-C₈)alkyl optionally substituted by at least one radical selected from the group consisting of: halogen, phenyl, nitro, cyano, and amino; (C₂-C₈)alkenyl optionally substituted by at least one radical selected from the group consisting of: halogen, nitro, cyano, and amino; (C₂-C₈)alkynyl optionally substituted by at least one radical selected from the group consisting of: halogen, nitro, cyano, and amino; and a radical which is a known ring system with 1-4 rings, wherein each one of the rings forming said ring system
has 3-7 members, each member independently selected from C, N, O, S, CH, CH₂, NH,
is saturated, partially unsaturated or aromatic, and
is partially/totally fused;
being each ring forming part of the ring system optionally substituted by at least one radical selected from the group consisting of: halogen, nitro, cyano, and amino;
for the manufacture of a medicament for the treatment of a disease mediated by a heat shock protein 90.

18. Use of a compound as defined in any of the claims 2-14
for the manufacture of a medicament for the treatment of a disease mediated by a heat shock protein 90.

19. Use according to any of the claims 17-18, wherein the disease mediated by a heat shock protein 90 is selected from the group consisting of: neoplasm, tumor, cancer, leukemia, psoriasis, bone diseases, proliferative condition; immunosuppressive conditions; arthritis; atherosclerosis; prion diseases and diseases associated with defects in protein folding and aggregation; viral diseases; and fibroproliferative disorders.

20. Use according to claim 19, wherein the disease mediated by a heat shock protein 90 is cancer.
